(19) United States
(12) Patent Application Publication
(10) Pub. No.: US 2014/0336187 A1
(43) Pub. Date: Nov. 13, 2014

(54) METHODS FOR TREATING LEISHMANIASIS

(71) Applicant: HOWARD UNIVERSITY, Washington, DC (US)

(72) Inventors: Oladapo Bakare, Beltsville, MD (US); Clarence M. Lee, Mitchellville, MD (US); Yakini Brandy, Hyattsville, MD (US); Cheu Manka, Washington, DC (US)

(21) Appl. No.: 14/359,035

(22) PCT Filed: Nov. 16, 2012
(86) PCT No.: PCT/US2012/065525

§ 371 Date: May 16, 2014

Related U.S. Application Data
(60) Provisional application No. 61/561,437, filed on Nov. 18, 2011.
(60) Provisional application No. 61/676,735, filed on Jul. 27, 2012.

(51) Int. Cl. C07C 225/30; C07D 207/408; C07D 265/33; C07D 209/48; C07C 233/33; C07C 233/92; C07C 235/88

(52) U.S. Cl. CPC C07C 225/30; C07C 233/92; C07D 207/408; C07C 235/88; C07D 209/48; C07C 233/33; C07D 265/33
USPC 514/230.8; 514/616; 514/657; 514/425; 514/417; 514/629

(57) ABSTRACT

Methods are provided to inhibit proliferation of Leishmania parasites, and in particular Leshmania donovani with imido-substituted 1,4-naphthoquinones, including novel compounds. Administering an imido-substituted 1,4-naphthoquinone can used to provide prophylaxis or treatment to a patient in need of treatment against leishmaniasis disease.

### RELATED APPLICATIONS

This PCT application claims priority from U.S. Provisional Applications 61/561,437, filed Nov. 18, 2011 and 61/676,735, filed Jul. 27, 2012, the complete disclosures of which are incorporated herein by reference.

### FIELD OF THE INVENTION

The present inventions relate to methods for inhibiting a parasite from the genus Leishmania, using an imido-substituted 1,4-napthoquinone compound.

### BACKGROUND OF THE INVENTION

Leishmaniasis is a parasitic infection caused by protozoan parasites of the genus Leishmania. The disease, named Leishman by the first person who described it in London in May 1903, is transmitted by the bite of a female sandfly (genus Phlebotomus) during a blood meal on its host. Clinical symptoms of the disease vary and include cutaneous, mucocutaneous, and visceral forms of the disease. Over 20 species and subspecies of Leishmania infect mammals, such as humans, causing a different spectrum of symptoms. These include Leishmania donovani complex with 2 species (Leishmania donovani, Leishmania infantum); the Leishmania mexicana complex with three main species (Leishmania mexicana, Leishmania amazonesis, and Leishmania venezuelensis); Leishmania tropica; Leishmania major; Leishmania aethiopica; and the subgenus Viannia with 4 main species Leishmania (V.) braziliensis, Leishmania (V.) guyanensis, Leishmania (V) panamensis, and Leishmania (V.) peruviana). Millions of people are at risk of disease and even death from the parasitic infections. It has been estimated approximately 12 million people worldwide are infected, 1.5 million with cutaneous leishmaniasis, 0.5 million with visceral leishmaniasis and that 350 million people are at risk of being infected. A conservative estimate of global yearly incidence is 1-1.5 million for cutaneous leishmaniasis and 0.5 million for visceral leishmaniasis.

Leishmaniasis is more common in tropical regions with warm climates making these regions most important areas for the historical development and concentration of zoonoses and related public health problems. In the western hemisphere (New World), it occurs in some parts of Mexico, Central America, and South America while in the Eastern Hemisphere (Old World) it is mostly found in regions of Asia, the Middle East, Africa, and Southern Europe (CDC, 2011). About 90% of visceral leishmaniasis (VL) cases occur in India, Bangladesh, Nepal, Sudan, Ethiopia, and Brazil while 90% of cutaneous leishmaniasis (CL) occurs in Afganisthan, Algeria, Iran, Saudi Arabia, Syria, Brazil, Colombia, Peru, and Bolivia. Cases of leishmaniasis found in the United States were mostly due to travel and immigration patterns. Cases in civilians are due to travelers acquiring the disease from tourist's destinations in Latin America. In the military, it is due to personnel becoming infected with leishmaniasis in Iraq and Afghanistan and returning home with infections (CDC, 2011).

In order to limit the number of cases of leishmaniasis in most endemic regions, the World Health Organization (WHO) in 2004 developed a plan of action in Afghanistan and its aim was to control debilitating leishmaniasis. WHO together with the Massoud Foundation and HealthNet International, in Kabul, Afghanistan, with the help of donations from the Belgian government, intended to reduce the incidence of leishmaniasis in less than two years. This initiative was again renewed in 2010 under the control of neglected tropical diseases and the major aim was to scale-up integrated interventions. This initiative, “working to overcome the global impact of neglected tropical diseases” covers 17 neglected tropical diseases mostly in poor settings where housing is below substandard, contamination of environments with filth is common, and insects and animals that spread disease are rampant (WHO, 2011). As a result of these initiatives, treatment with preventive chemotherapy reached 670 million people in 2008, however the data related to leishmaniasis have not been updated (WHO, 2010).

Even though some efforts to reduce vector and mammalian reservoir populations have been successful, no vaccines have been developed for leishmaniasis as yet (CDC, 2010). In some cases, there is reduced responsiveness. Patients who used to respond effectively to drugs suddenly fail to respond or relapse.

Treatment of leishmaniasis is by the use of pentavalent antimonials. Sodium stibogluconate is mostly used in several endemic regions for the treatment of all three types of leishmaniasis. However, it has a problem of drug resistance. Amphotericin B (Am.B), aminosidine (paromomycin, gabbromicina), pentamidine are used for all forms of leishmaniasis while miltefosine is an available treatment option for visceral leishmaniasis. The orphan drug Aminosidine, is mostly available in the United States while miltefosine is an approved first line drug in India.

Available drugs for the treatment of Leishmania are very expensive, present resistance, show less responsiveness with continuous use for treatment, and are highly cytotoxic to infected individuals throughout endemic regions. There are currently four to six available drugs for the treatment of leishmaniasis, but they are all toxic, expensive and most often, are not effective. Oftentimes the drugs are simply ineffective.

Even during treatment, infected individuals are required to take some time off from work to complete treatment due to toxic effects of current drugs and this is a big obstacle to economic growth.

Am. B in combination with miltefosine has resulted in greater than 90% cure rates of visceral leishmaniasis in north India. However, the major problem is that of toxicity, high cost, resistance, primary unresponsiveness, and lower sensitivity still exist.

Therefore, identification of alternative candidate compounds with anti-Leishmanial activities is of utmost urgency, and in particular there is a critical need to develop new therapeutic agents that have low cytotoxicity but high effectiveness against Leishmania parasites.

### SUMMARY OF THE INVENTION

In its broadest aspect, a method for treating a mammalian patient at risk or suffering from a disease caused by a kinetoplastid parasite comprises administering to such subject an effective kinetoplasticidal amount of an imido-substituted 1,4-naphthoquinone to inhibit the kinetoplastid.

In an important aspect, a method of inhibiting Leishmania comprises administering to a patient for prophylaxis or to a patient in need of treatment an anti-Leishmanial effective amount of an imido-substituted 1,4-naphthoquinone.

In another important aspect, a method of inhibiting Leishmania comprises administering to a patient for prophylaxis or to a patient in need of treatment an anti-Leishmanial effective amount of an imido-substituted 1,4-naphthoquinone represented by the general formula:

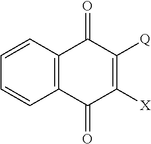

wherein X is hydrogen, halogen, alkoxy (cyclic or alicyclic), trifluoro methyl, aryloxy, or benzyloxy; and Q represents the imido-substituent bonded to the 1,4-napthoquinone moiety through the imido nitrogen.

In an aspect of the method, in the general formula X is bromo, chloro, fluoro or iodo.

In an aspect of the method, in the general formula, X is bromo or chloro.

In an aspect of the method, Q is represented by:

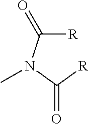

wherein in Q each R is, independently, a substituted or unsubstituted hydrocarbon, provided that one R can, optionally, be hydrogen, and provided that, optionally, R can include at least one hetero atom.

In an aspect of the method, Q is represented by:

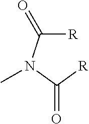

wherein in Q each R is, independently, cyclic or acyclic, substituted or unsubstituted, or the R groups bond together to form a cyclic imido-substituent.

In an aspect of the method, when Q is represented by:

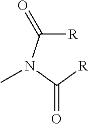

wherein each R is independent of the other, and
(a) R is an optionally substituted straight, branched or cyclic alkyl group, wherein the substitution is, for example, halogen, alkoxy or acetoxy,
(b) the R groups bond together to form an alkylene group whereby Q is a cyclic imido-substituent,
(c) the R groups bond together to form an alkylene group having a hetero atom, or
(d) R is aryl or substituted aryl, wherein the substitutent(s) include, for example, alkoxy or halogen.
   When R is cycloalkyl or aryl, a ring can include 0, 1, 2, 3, 4, or 5 substituents. Each R is independent of the other. In principle, one R can be hydrogen.

In an aspect of the method, Q is an aryl-imido substituent.

In an aspect of the method, the imido-substituted 1,4-naphthoquinone in the general formula is represented by:

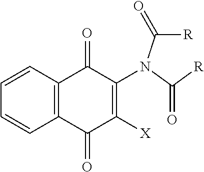

wherein each R is, independently, an optionally halo-substituted straight or branched C₁ to C₁₀ alkyl, preferably a C₁ to C₆ alkyl. X is hydrogen, halogen, alkoxy (cyclic or alicyclic), trifluoro methyl, aryloxy, or benzyloxy.

In an aspect of the method, Q in the general formula is represented by:

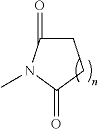

wherein the symbol ( ) designates —(CH₂)— and n is 1 to 3. Preferably n is 1 or 2.

In an aspect of the method, Q is represented by:

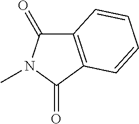

wherein the aryl ring may, optionally, be substituted, such as substituted with halogen.

In an aspect of the method, an imido-substituted 1,4-naphthoquinone is represented by:

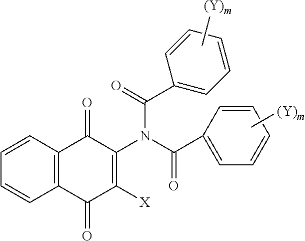

wherein X is hydrogen, halogen, alkoxy (cyclic or alicyclic), trifluoro methyl, aryloxy, or benzyloxy, to mention examples; each Y, independently, represents hydrogen, halogen, alkoxy (cyclic or alicyclic), trifluoro methyl or alkyl, and each m, independent of the other, is 0, 1, 2, 3, 4 or 5.

In an aspect of the method, on one of the aryl rings each Y can be hydrogen.

In an aspect of the method, when each m is 0, an imido-substituted 1,4-naphthoquinone is represented by:

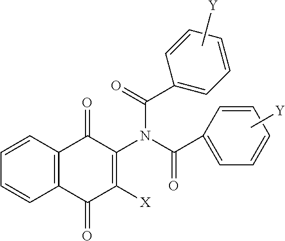

wherein each Y is hydrogen.

In an aspect of the method, an imido-substituted 1,4-naphthoquinone is represented by:

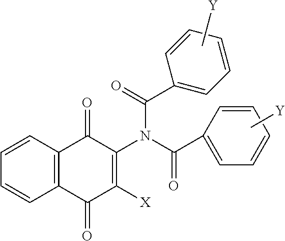

wherein X is hydrogen, halogen, alkoxy (cyclic or alicyclic), trifluoro methyl, aryloxy, or benzyloxy, and each Y, independently, is alkoxy, aryloxy or halogen, and each m is 1. Y is ortho, meta or para-substituted. In another aspect, Y is meta-substituted. In a further aspect of this method, one Y can be hydrogen.

In an aspect of the method, an imido-substituted 1,4-naphthoquinone is represented by:

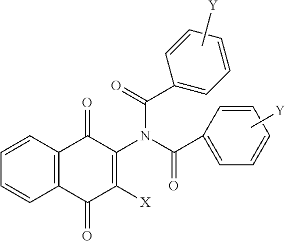

wherein X is hydrogen, halogen, alkoxy (cyclic or alicyclic), trifluoro methyl, aryloxy, or benzyloxy and each Y, independently, is halogen. X and Y are independent of each other. In a further aspect, Y is ortho, meta or para-substituted. In another aspect, Y is meta-substituted. In a further aspect of this method, one Y can be hydrogen.

In an aspect of the method, an imido-substituted 1,4-naphthoquinone having in vitro toxicity against both promastigote and amastigote forms of Leishmania donovani parasites is administered to a patient in need of treatment.

In an aspect of the method, an imido-substituted 1,4-naphthoquinone having a selectivity index against both promastigote and amastigote forms of Leishmania donovani parasites is administered to a patient in need of treatment.

In an aspect of the method, an imido-substituted 1,4-naphthoquinone having an IC₅₀ cytoxicity value less than that for Amphotericin B, is administered to a patient in need of treatment against Leishmania parasites.

In an aspect of the method, an anti-Leishmanial effective amount of a compound selected from the group consisting of IMDNQ2, IMDNQ3, IMDNQ4, IMDNQ5, IMD8 and IMNDQ15 is administered to a patient to inhibit Leishmania donovani.

In an aspect of the method, IMDNQ2, IMDNQ3, IMDNQ4, IMDNQ5, IMD8 and IMDNQ15 had IC₅₀ values of 2.27 μM, 10.81 μM, 6.81 μM, 31.28 μM, 4.3 μM, and 0.05 μM in promastigotes and 5.83 μM, 4.10 μM, 1.19 μM, 4.67 μM, 2.07 μM, 18.85 μM in amastigotes, respectively.

In an aspect, IC₅₀ values of IMDNQ2, IMDNQ3, IMDNQ4, IMDNQ5, IMD8 and IMNDQ15 in mice fibroblasts cells were much higher (78.75 μM, 24.83 μM, 168.1 μM, 4.7 μM, 14197.35 μM, and 2.94 μM, respectively) compared to that of Amphotericin B (known drug for treating leishmaniasis) which was 1.18 μM.

In an aspect, IMD7, IMD8, IMDNQ4, IMDNQ2, and IMDNQ3 exhibited very low cytotoxicity, with IC₅₀ values being 1448.56 μM, 14197.35 μM, 168.1 μM, 78.75 μM, and 24.83 μM against mouse fibroblasts cells.

In an aspect, the method comprises treating a patient in need of treatment for leishmaniasis with a therapeutically effective amount of an active ingredient that is a compound represented by the general formula.

In an aspect, the method comprises treating a patient in need of treatment for leishmaniasis with a therapeutically effective amount of an active ingredient that is a compound selected from the group consisting of IMDNQ1, IMDNQ2, IMDNQ3, IMDNQ4, IMDNQ5, IMDNQ6, IMD7, IMD8, IMDNQ9, IMNDQ10, IMNDQ11. IMNDQ12, IMNDQ13, IMNDQ14 and IMNDQ15.

In an aspect, the method comprises prophylaxis against Leishmania genus by administering an effective anti-Leishmanial amount of a compound represented by the general formula to a patient.

In an aspect, the method comprises prophylaxis against Leishmania genus by administering an effective anti-Leishmanial amount of an active ingredient that is a compound selected from the group consisting of IMDNQ1, IMDNQ2, IMDNQ3, IMDNQ4, IMDNQ5, IMDNQ6, IMD7, IMD8, IMDNQ9, IMNDQ10, IMNDQ11. IMNDQ12, IMNDQ13, IMNDQ14 and IMNDQ15 or a derivative thereof.

In an aspect, the method comprises inhibiting tublin polymerization in a Leishmania parasite by administering an effective anti-tublin polymerization amount of an imido-substituted 1,4-naphthoquinione compound. In a further aspect the compound is represented by the general formula. In a further aspect the parasite is Leishmania donovani.

### BRIEF DESCRIPTION OF THE FIGURES

FIG. 1 includes structures of certain imido-substituted 1,4-naphthoquinone compounds having in vitro activity against Leishmania donovani parasites.

FIG. 2 is a bar graph showing the cytotoxicity of certain imido-substituted 1,4,-naphthoquinone compounds on Balb/C 3T3 mouse fibroblasts.

FIG. 3 is a bar graph showing the anti-Leishmanial activity of certain imido-substituted 1,4,-naphthoquinone compounds in Leishmania donovani promastigotes and amastigotes.

FIG. 4 is a bar graph showing selectivity indices (SI) of certain imido-substituted naphthoquinone compounds in Leishmania donovani promastigotes and amastigotes.

FIG. 5A-X shows structures of exemplary compounds for reacting with a suitable 1,4-naphthoquinone starting material to obtain an imido-substituted 1,4-napthoquinone.

FIG. 6 is a bar graph showing antileishmanial activities of 15 imido-substituted naphthoquinone compounds against L. donovani promastigotes.

FIG. 7 is a bar graph showing antileishmanial activities of 15 imido-substituted naphthoquinone compounds against L. donovani amastigotes.

FIG. 8 is a bar graph showing cytotoxicity of 15 imido-substituted naphthoquinone compounds on NIH 3T3 BALB/c mouse fibroblast cell line in vitro.

FIG. 9 is a bar graph showing selectivity indices (SIs) of imido substituted naphthoquinone compounds in L. donovani promastigotes.

FIG. 10 is a bar graph showing selectivity indices (SIs) of imido substituted naphthoquinone compounds on L. donovani amastigotes.

FIG. 11 is a bar graph showing two hours exposure of imido-substituted naphthoquinone compounds to promastigotes.

FIG. 12 is a bar graph showing four hours exposure of compounds to promastigotes.

FIG. 13 is a bar graph showing six hours exposure of compounds to promastigotes.

FIG. 14 is a bar graph showing twenty four hours exposure of compounds to promastigotes.

FIG. 15 shows a comparison of liver and spleen imprints of BALB/c Mice before and after infection with promastigotes of L. donovani.

FIG. 16 is a bar graph showing the effects of VL infection on serum IgG levels.

FIGS. 17A-D are bar graphs showing treatment of BALB/c mice with selected naphthoquinone compounds.

FIG. 18 is a bar graph showing alanine aminotransferase AST levels assessment on VL BALB/c mice model.

FIG. 19 is a bar graph showing effects of compounds on alanine aminotransferase (ALT) levels.

### DETAILED DESCRIPTION OF THE INVENTION

The methods described herein advantageously utilize imido-substituted 1,4-naphthoquinones as a novel class of anti-Leishmanial agents to inhibit proliferation of Leishmania parasites. The methods can provide prophylaxis or treatment for a vertebrate against a parasite in the Leishmania genus. The methods can provide treatment against the various stages of Leishmania parasite infections. Thus, administering an imido-substituted 1,4-naphthoquinone can provide prophylaxis or treatment to a patient against the proliferation of Leishmania parasites.

In particular, the method can provide treatment for a human against Leishmania disease.

Administering an imido-substituted 1,4-naphthoquinone to a patient in a stage of infection with Leishmania genus can treat against cutaneous, mucocutaneous, and visceral forms of the disease.

Administering an imido-substituted 1,4-naphthoquinone to a patient in a stage of infection with Leishmania donovani can treat against visceral leishmaniasis.

Administering an imido-substituted 1,4-naphthquinone to a patient in a stage of infection with Leishmania donovani can treat against promastigote and/or amastigote forms of Leishmania donovani parasites.

Administering an imido-substituted 1,4-naphthoquinone can be used to treat Leishmania infections where the parasites are susceptible in promastigote and/or amastigote forms of Leishmania donovani within the life cycle.

Administering for prophylaxis may help break the life cycle of leishmaniasis disease and reduce the patient's chances of becoming infected or infecting another through a vector.

Administering an imido-substituted 1,4-naphthoquinone to a patient can, in principle, lead to inhibiting proliferation of Leishmania, by directly affecting the parasite's life cycle. Once a vector ingests blood from the patient whose blood plasma contains a imido-substituted 1,4-naphthoquinone, further development of Leishmania parasite in the vector may be inhibited.

The imido-substituted 1,4-naphthoquinones include, for example, 2-imido 3-halo-1,4-naphthoquinones.

An aspect of the method is inhibiting proliferation of Leishmania in a patient in need of treatment by administering a cyclic-imido-substituted 1,4-naphthoquinone, to the patient.

Administering includes sublingual administration, oral administration, and, in principle, intravenous administration. A pharmaceutical composition can contain the active pharmaceutical ingredient and may additionally comprise a pharmaceutically acceptable vehicle or adjuvant. A pharmaceutical composition can be in the form of a solid pharmaceutical dosage form (tablet, caplet, capsule, or deliverable from an osmotic pump as examples) or syrup. Remington, The Science and Practice of Pharmacy, provides general information regarding pharmaceutical dosage forms.

An anti-Leishmanial effective amount of the imido-substituted 1,4 naphthoquinone refers to an amount effective in inhibiting proliferation of a parasite in the Leishmania genus and includes an leishmaniocidal amount against a parasite from the Leishmania genus.

A therapeutically effective amount means an amount of the imido 1,4-naphthoquinone that can provide a therapeutic benefit to a patient against leishmaniasis.

Patient includes human. A patient in need of treatment includes a human patient in need of treatment against Leishmania parasites. Thus, methods of treating a mammal other than human (veterinary treatments) against Leishmania parasites are also within the scope of our inventions, and in particular canines.

A method for inhibiting proliferation of Leishmania with imido-substituted 1,4-naphthoquinones, such as imido-substituted 3-halo 1,4-napthoquinones, can exhibit greater anti-Leishmanial efficacy against Leishmania than the presently clinically used standard drug, Amphotericin B. For example, compared to Amphotericin B (IC₅₀=5.26 μM in promastigotes and 22.26 μM in amastigotes), some imido-naphthoquinone analogs (as examples) are significantly more potent against Leishmania, such as IC₅₀ values ranging from IMDNQ4 having 1.19 μM in amastigotes and IMDNQ15 having 0.5 pM in promastigotes. Thus, in one of its aspects the method for inhibiting proliferation of Leishmania comprises administering an imido-substituted 1,4-naphthoquinone, such as an imido-substituted 3-halo 1,4-napthoquinone, having an acceptable IC₅₀ value, preferably an IC₅₀ value equal to or lower than Amphotericin B.

A method for inhibiting proliferation of Leishmania, and in particular Leishmania donovani with an imido-substituted 1,4-naphthoquinone, especially an imido-substituted 3-halo 1,4-napthoquinone, can exhibit greater selectivity against Leishmania than the presently clinically used Amphotericin B. Thus, in another of its aspects the method for inhibiting proliferation of Leishmania comprises administering an imido-substituted 1,4-naphthoquinone, such as an imido-substituted 3-halo 1,4-napthoquinone, having an acceptable selectivity index, preferably a selectivity index better than Amphotericin B.

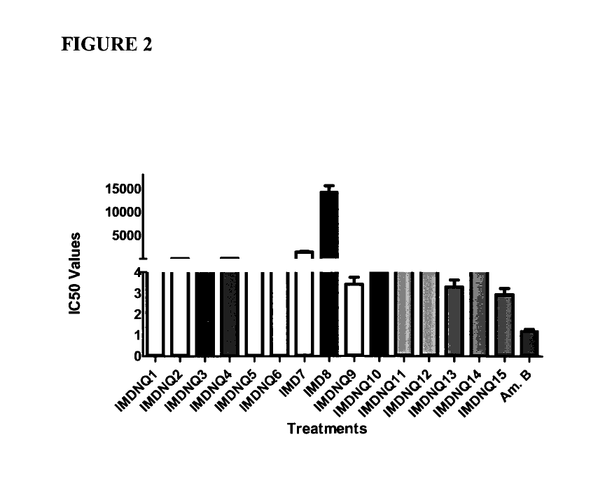

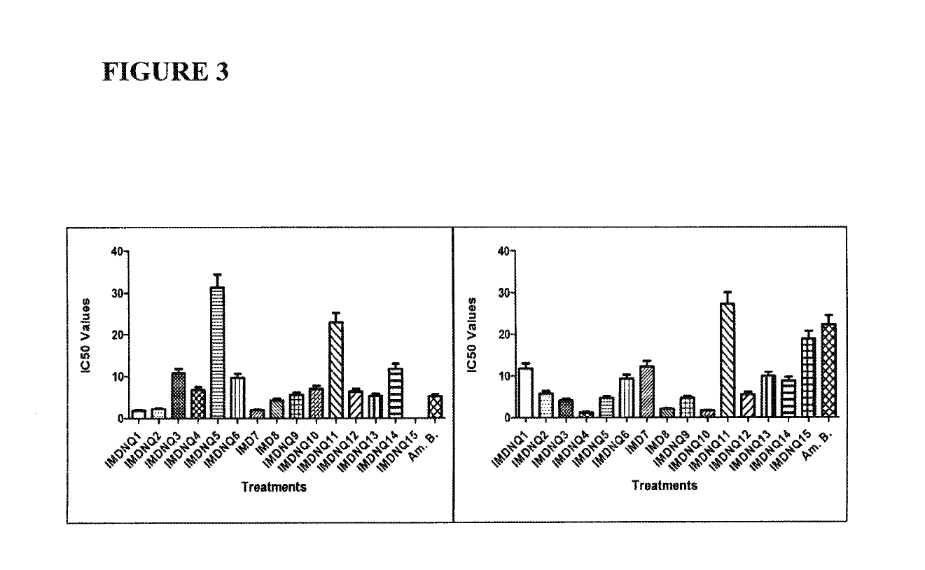

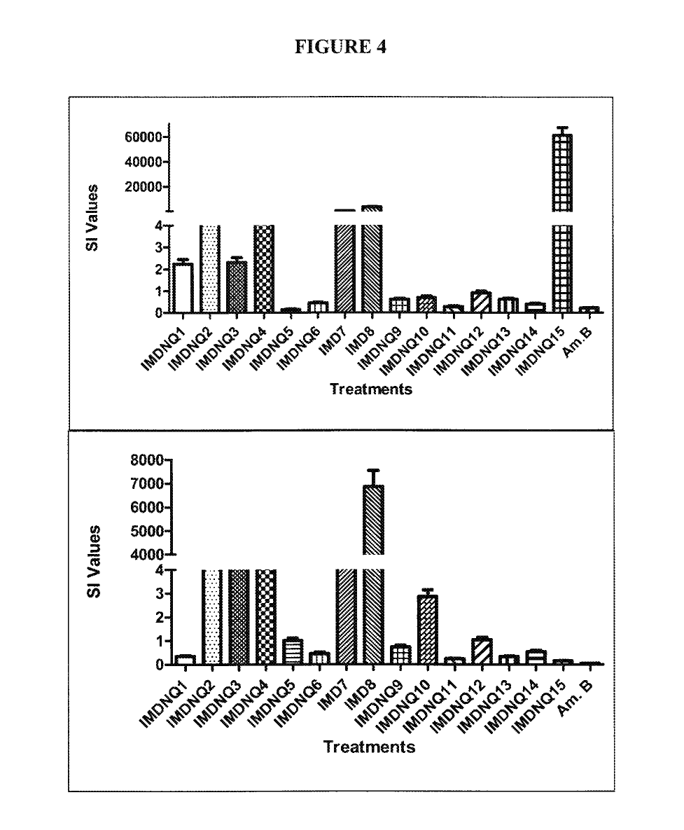

A method for inhibiting proliferation of Leishmania with an imido-substituted 1,4-naphthoquinone, such as an imido-substituted 3-halo 1,4-napthoquinone, exhibiting better cyotoxicity characteristics than Amphotericin B. In vitro testing has demonstrated representative imido-naphthoquinone analogs were relatively non-cytotoxic to Balb/C 3T3 mouse fibroblast cell line with IC₅₀ values of well over the value for Amphotericin B. For example, cytotoxicity study on Balb/C 3T3 mouse fibroblast cell line showed that IMDNQ4, IMD7, and IMD8 are far less cytotoxic than Amphotericin B (FIG. 2). Thus, in yet another of its aspects the method for inhibiting proliferation of Leishmania comprises administering an imido-substituted 1,4-naphthoquinone, such as an imido-substituted 3-halo 1,4-napthoquinone, having an acceptable cytotoxicity value, preferably a value better than Amphotericin B.

In the synthesis of the imido-substituted 1,4-naphthoquiniones, compounds represented by the formula:

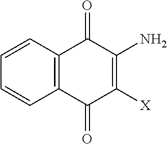

wherein X is hydrogen, halogen, alkoxy (cyclic or alicyclic), trifluoro methyl, aryloxy, or benzyloxy, are suitable starting materials that provide the 1,4-napthoquinone skeleton. For example, a 2-amino-3-halo-1,4-naphthoquinone is a suitable starting material for preparing imido-substituted 1,4, naphthoquiones having a 3-halo 1,4-naphthoquinone skeleton. The 2-amino-3-chloro-1,4-naphthoquinone is commercially available. It can also be facilely obtained from 2,3-dichloro-1,4-naphthoquinone and ammonia in a mixture of concentrated ammonium hydroxide and ethanol. A 2-amino-3-bromo-1,4-naphthoquinone starting material can be prepared by refluxing commercially available 2,3-dibromo-1,4-naphthoquinone with ammonia/ammonium hydroxide mixture in ethanol. A 2-amino-3-iodo-1,4-naphthoquinone starting material can be prepared as described in Perez et al., Synthesis of Iodinated Naphthoquinones Using Morpholine-Iodine Complex, Synthetic Communications, 34(18):3389-3397 (2004) (compound (14)), the complete disclosure of which is incorporated herein by reference. For imido-substituted 1,4, naphthoquiones having a 2-alkoxy 1,4-naphthoquinone skeleton, a 2-amino-3-alkoxy-1,4-naphthoquinone or 2-amino 3-aryloxy-1,4-naphthoquinone are representative classes of starting material. It will be appreciated that X can also be halo-alkyl, such as trifluoro methyl, or halo-alkoxy, such as trifluoromethoxy or a halo-alkyl, such as trifluoro methyl as an example.

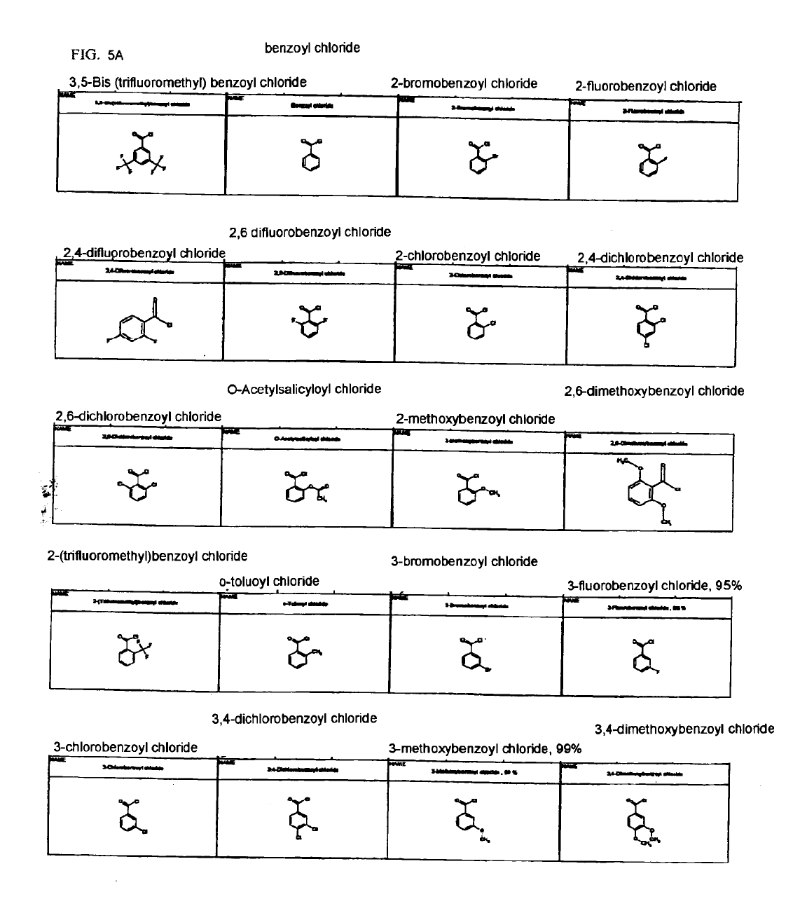

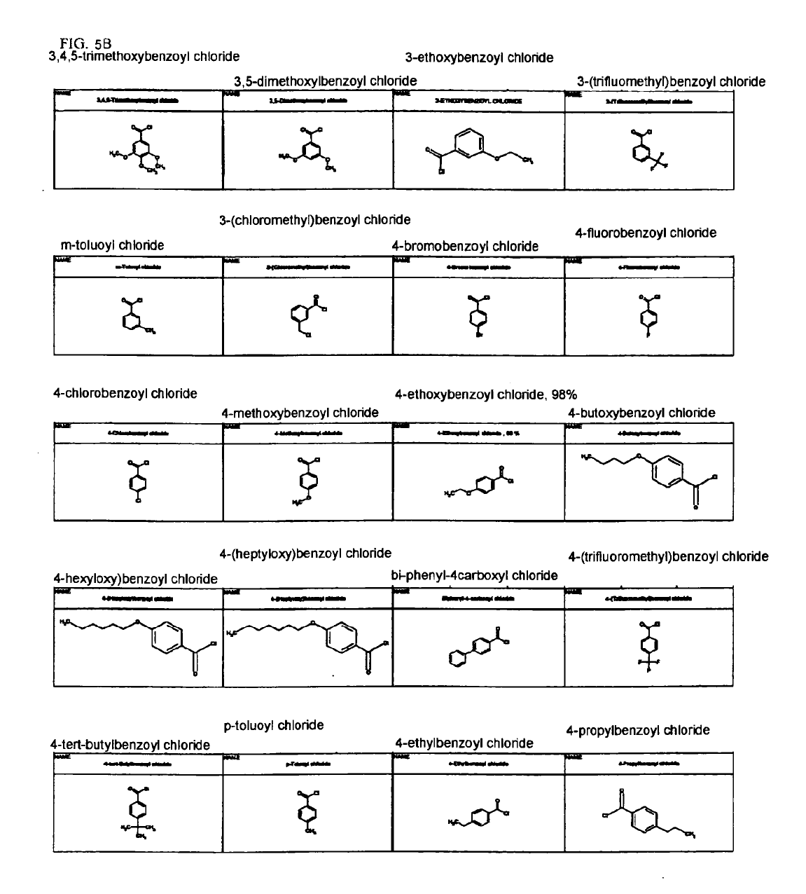

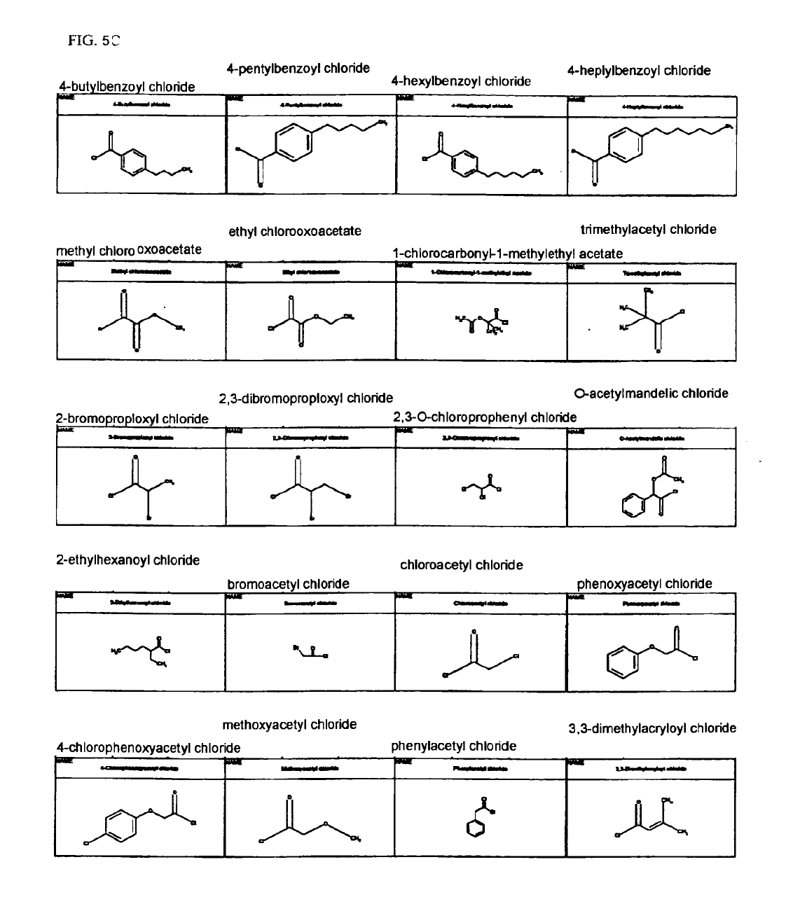

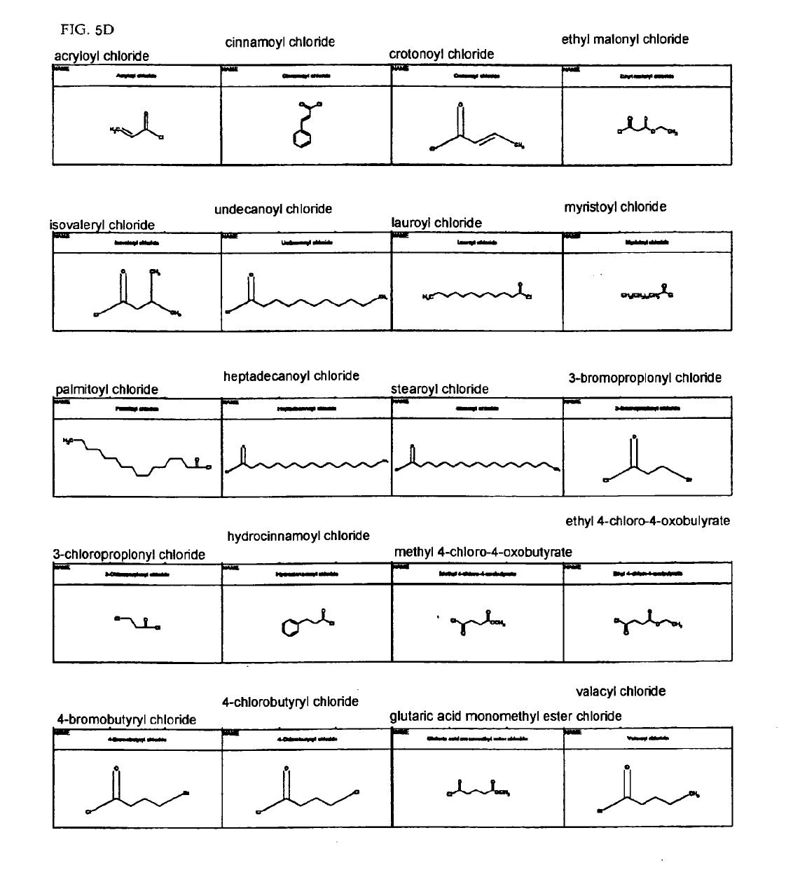

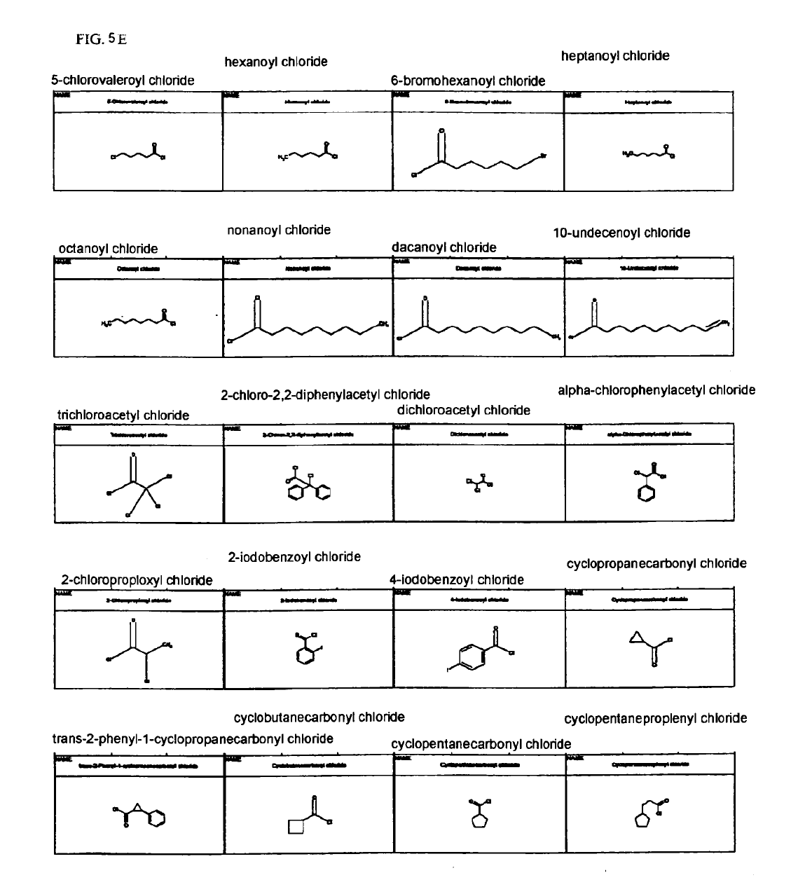

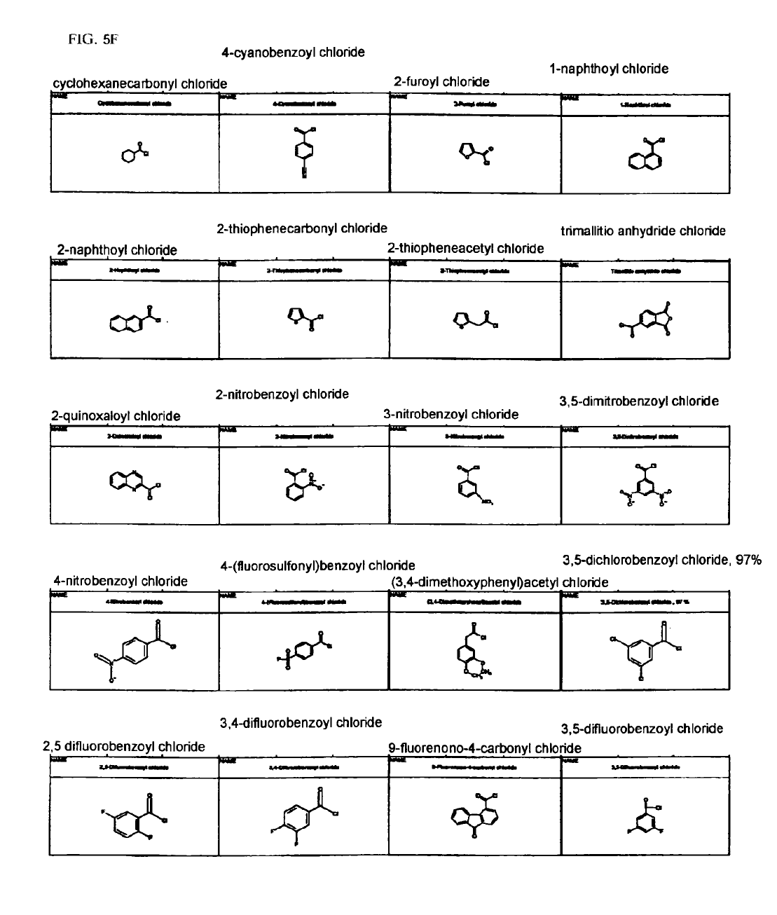

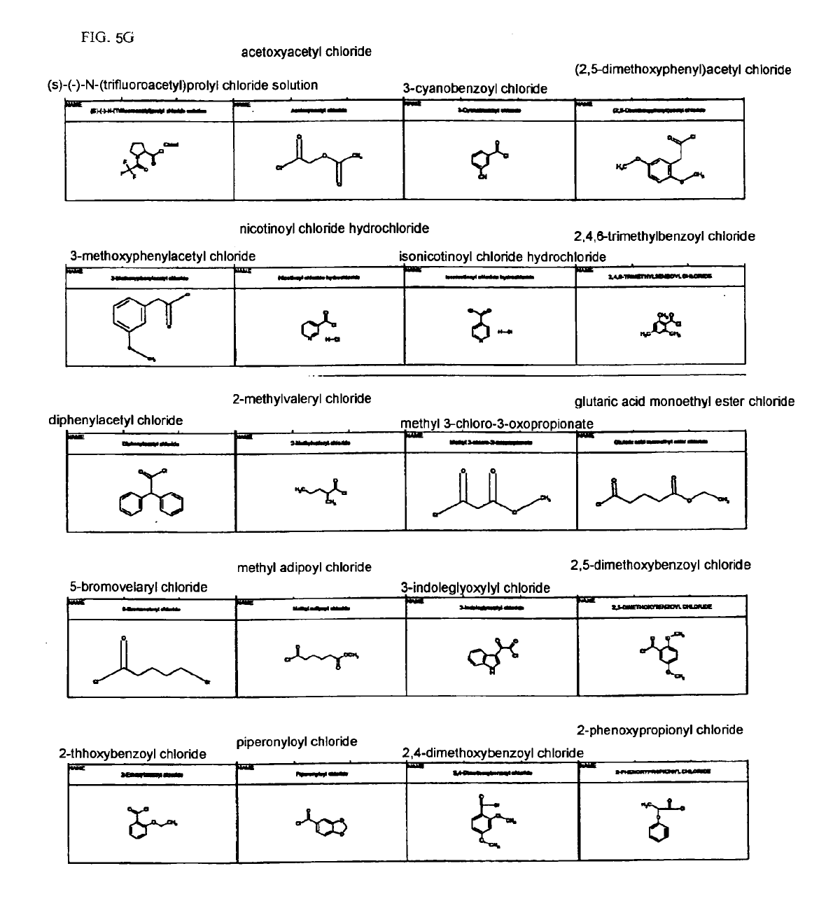

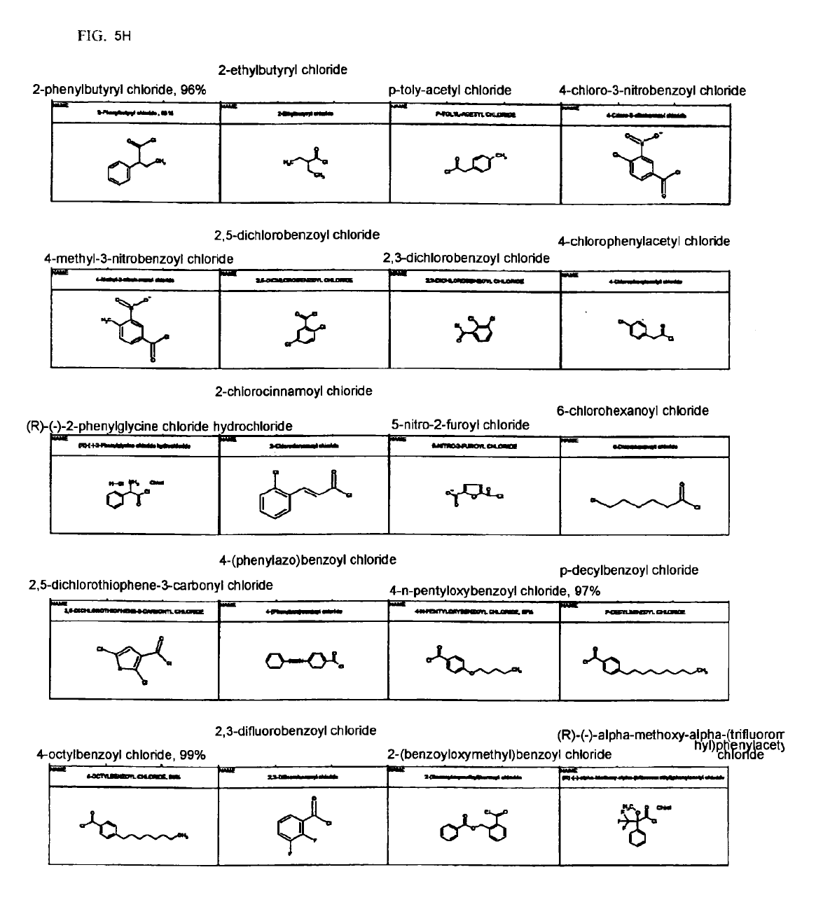

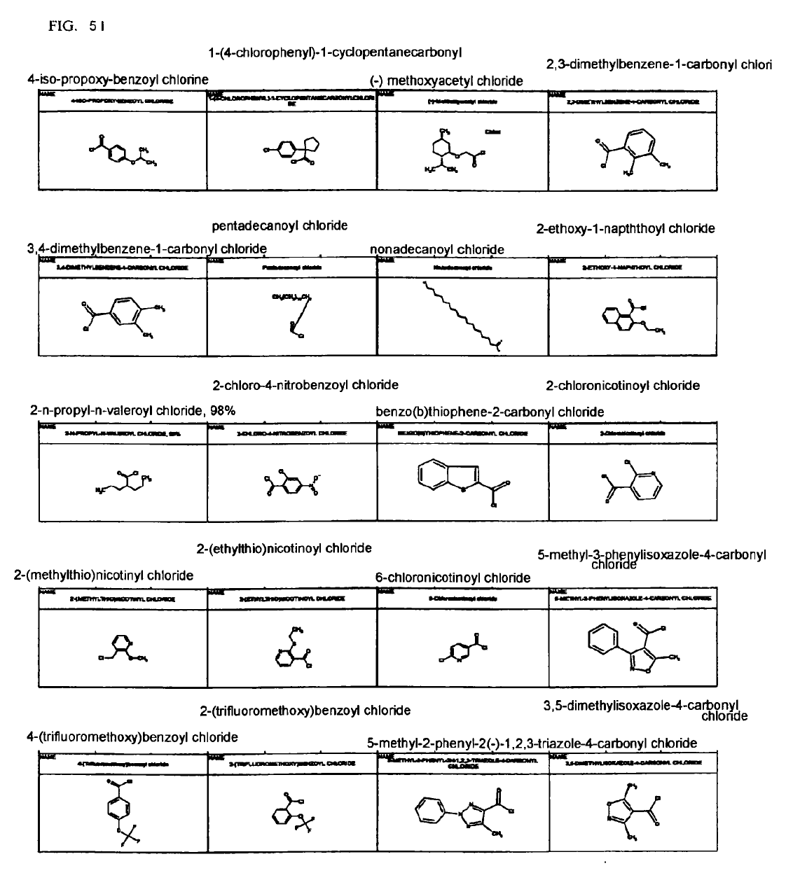

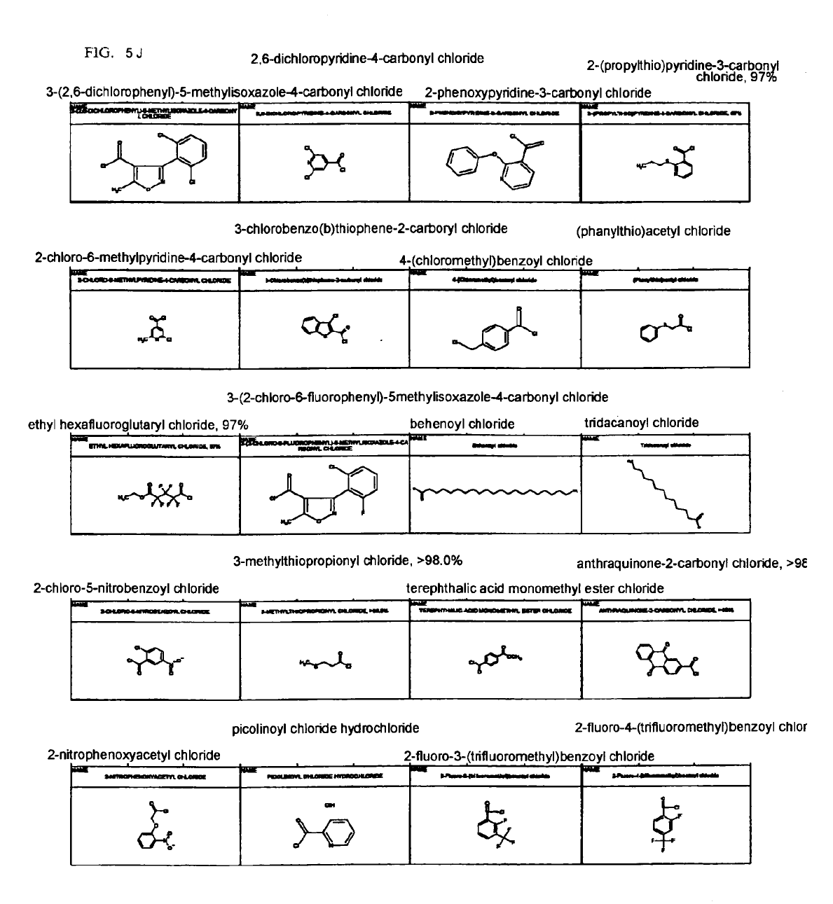

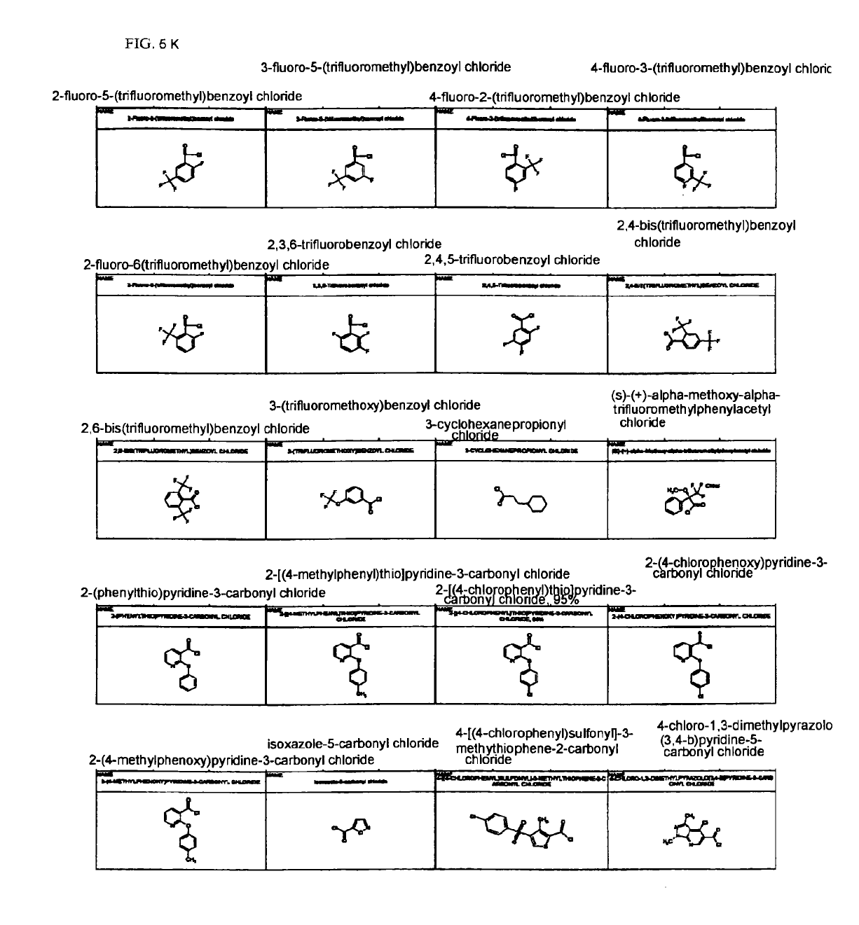

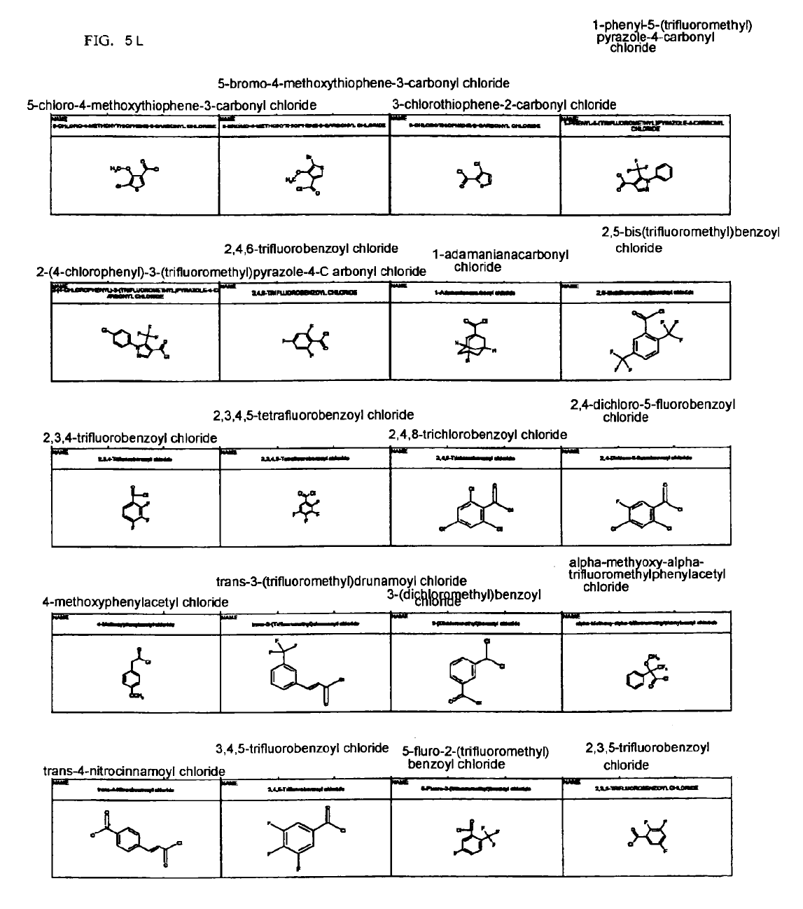

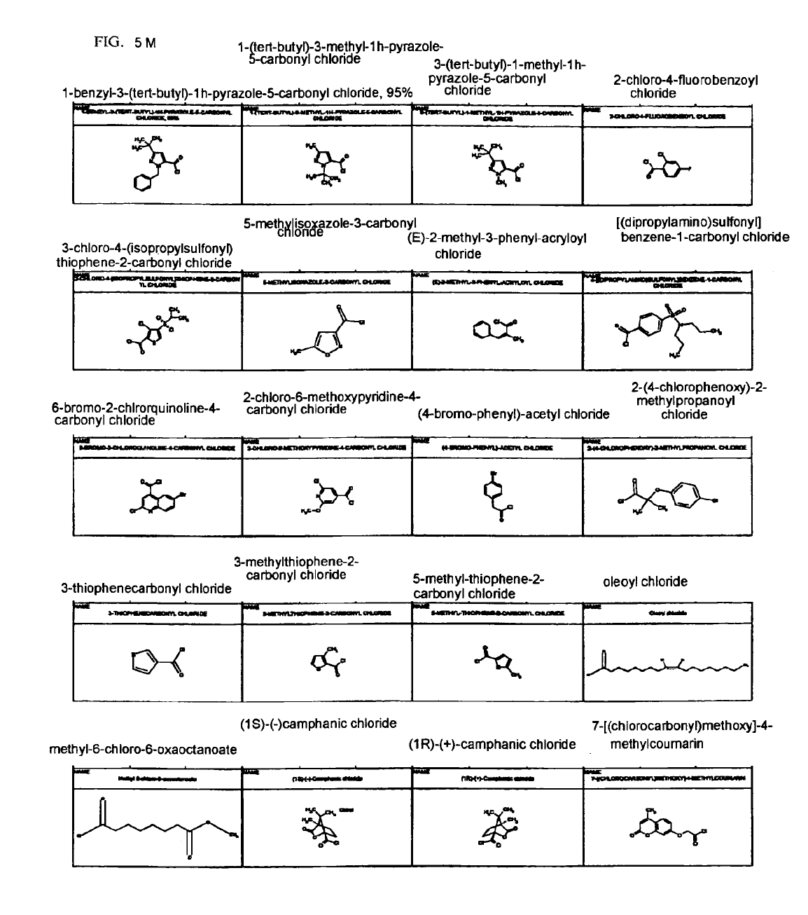

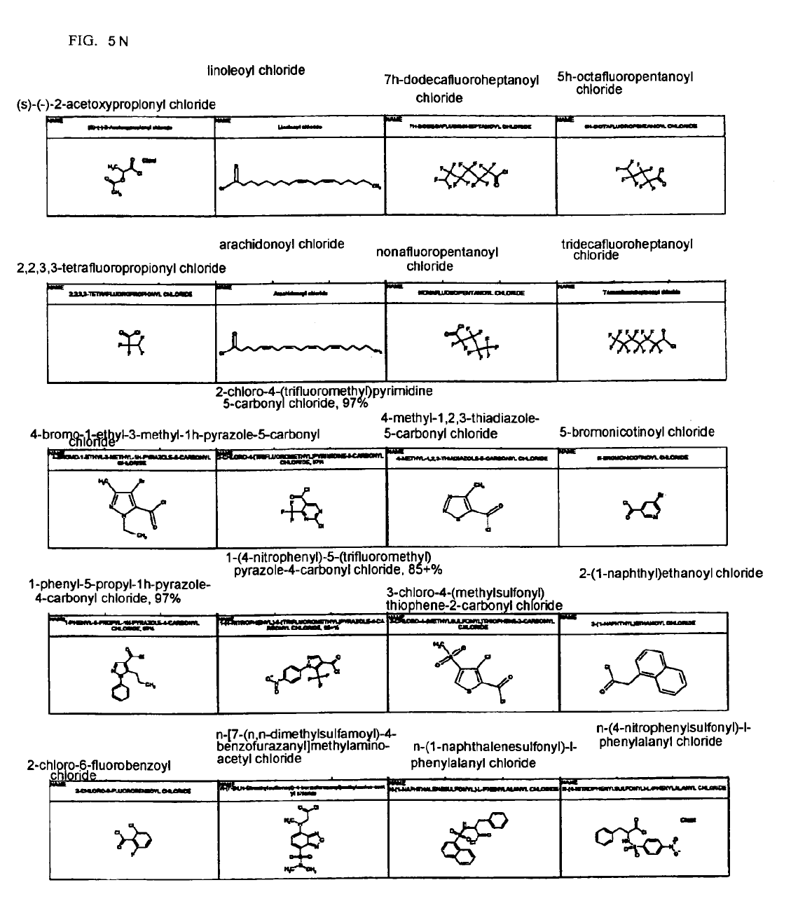

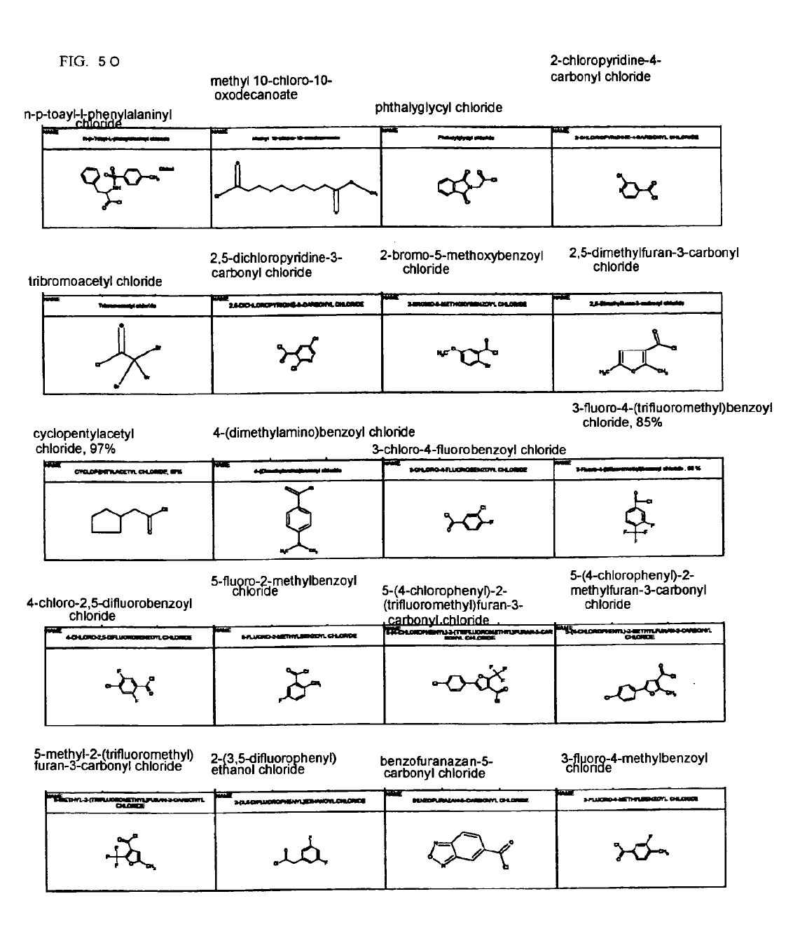

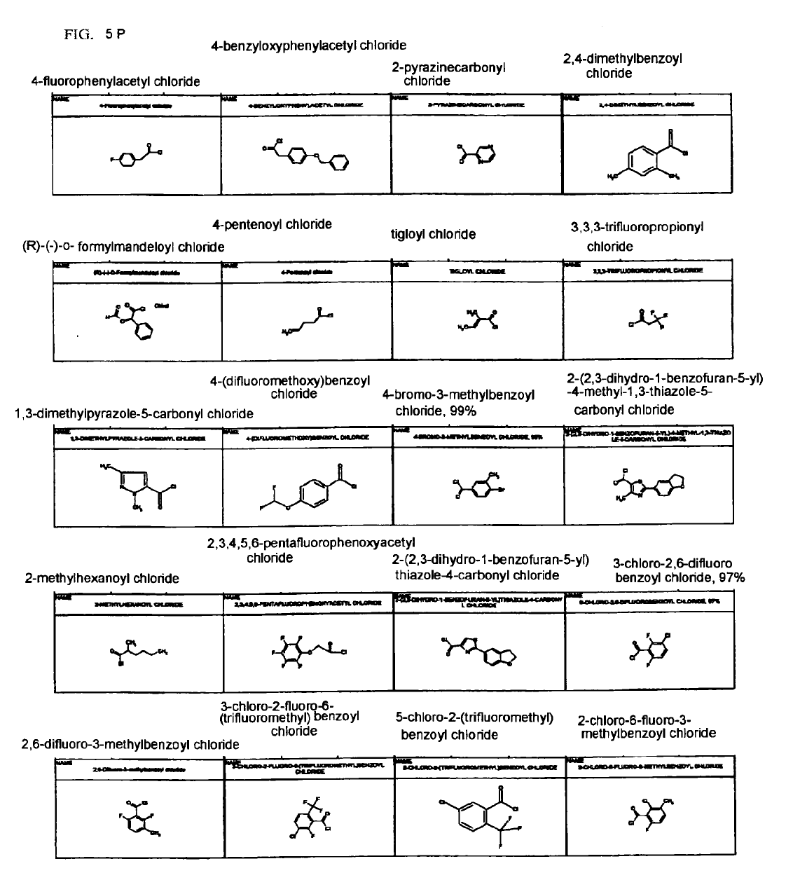

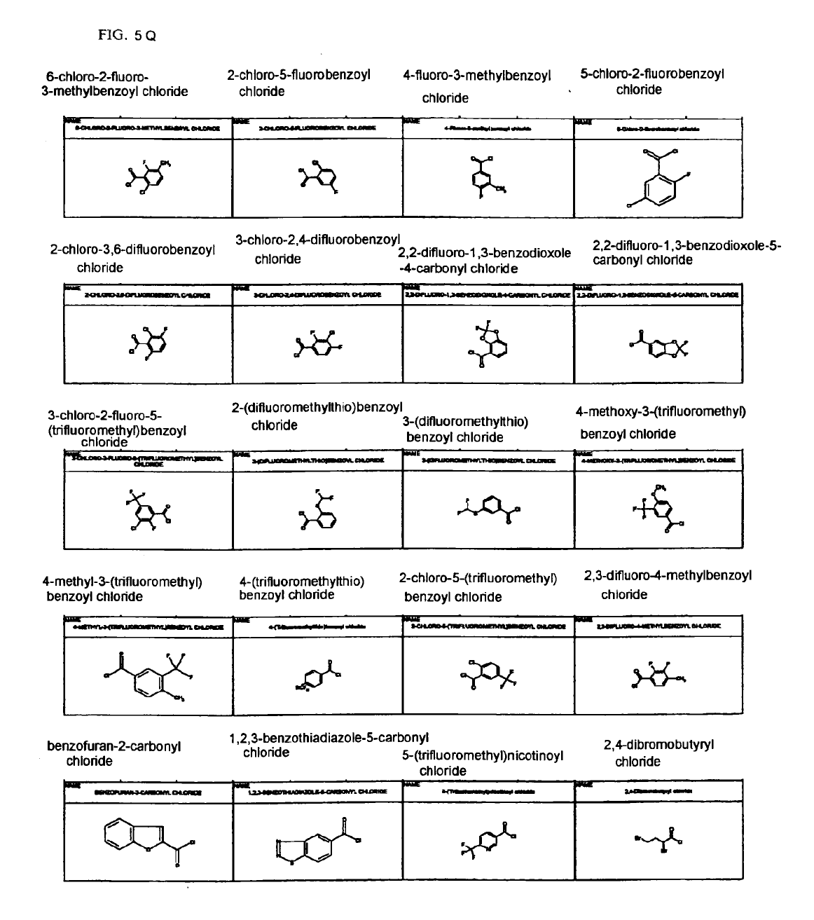

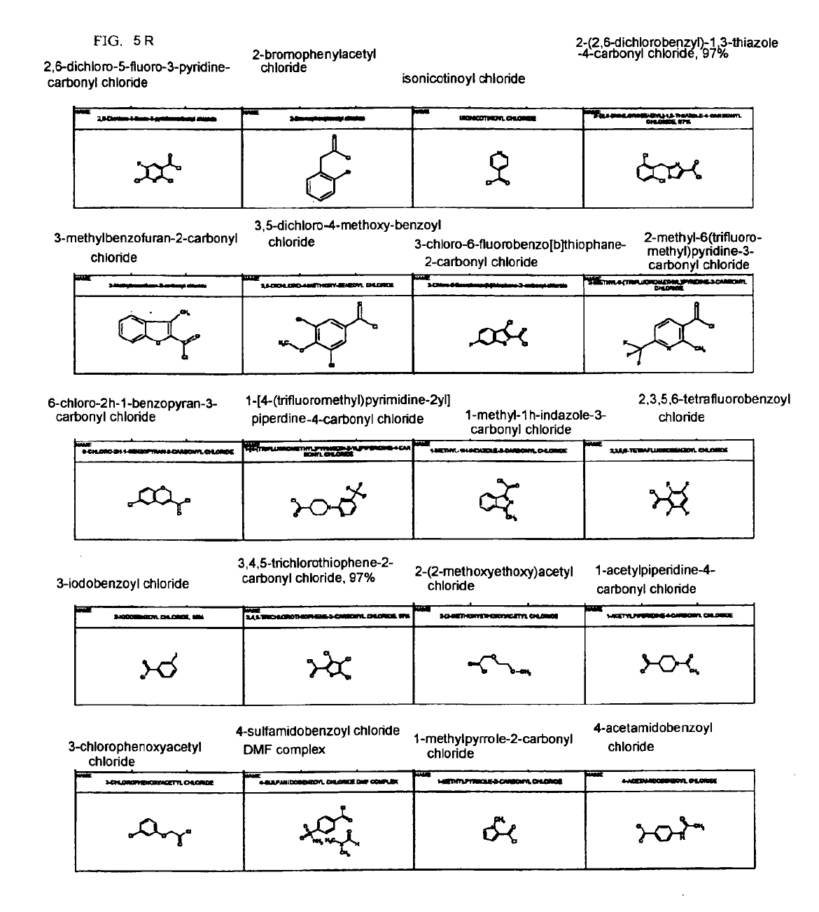

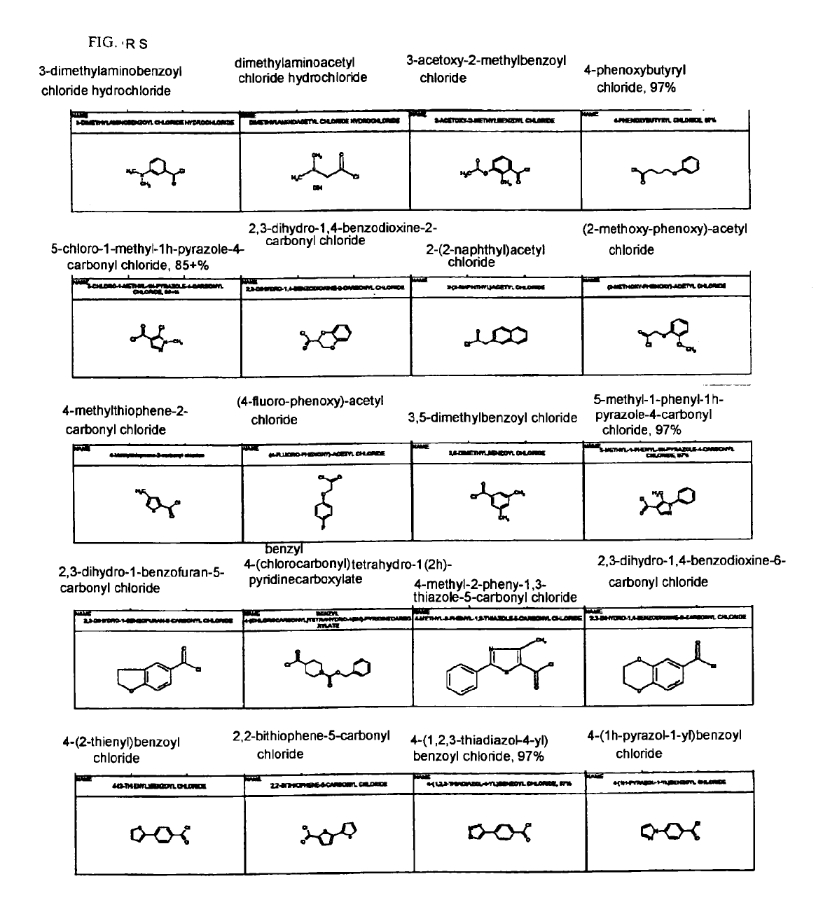

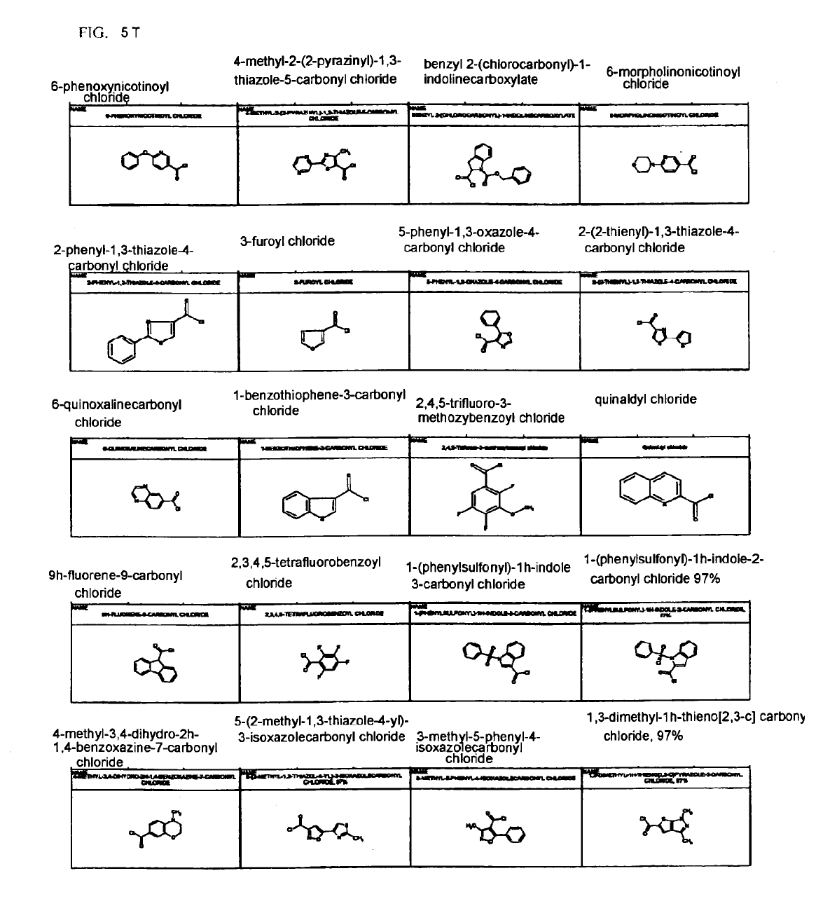

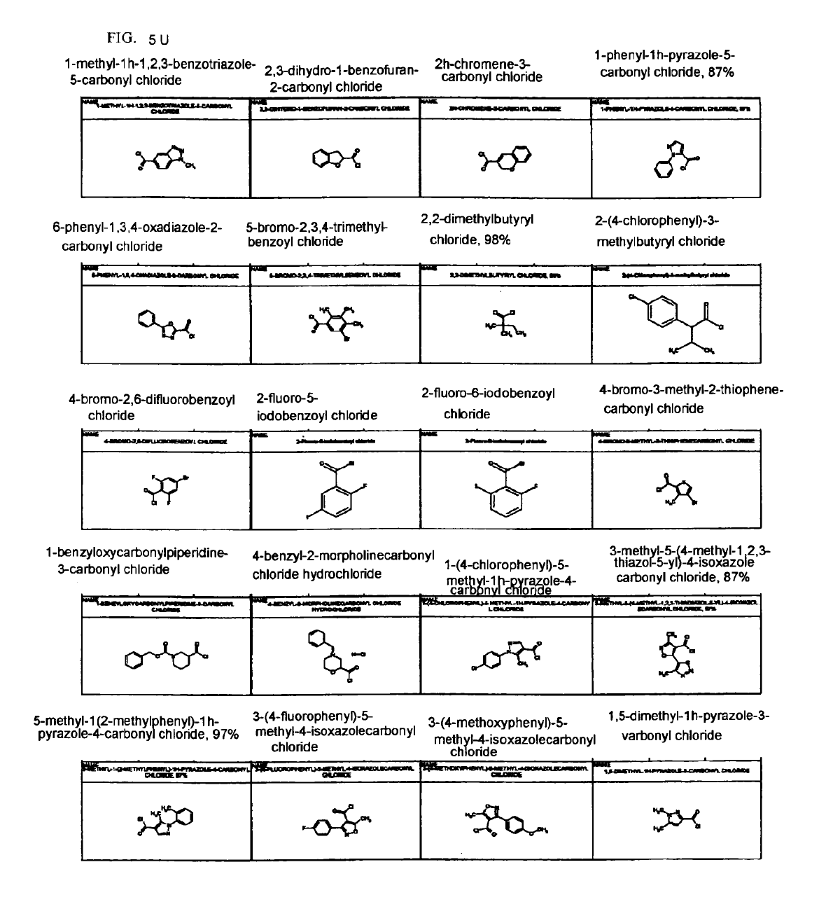

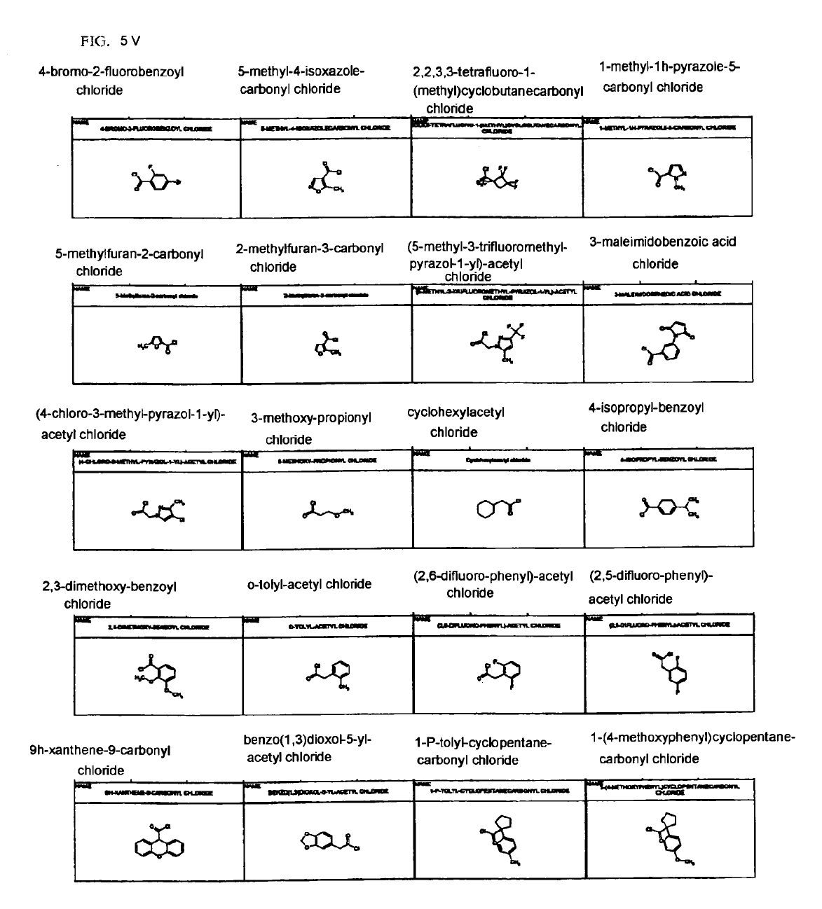

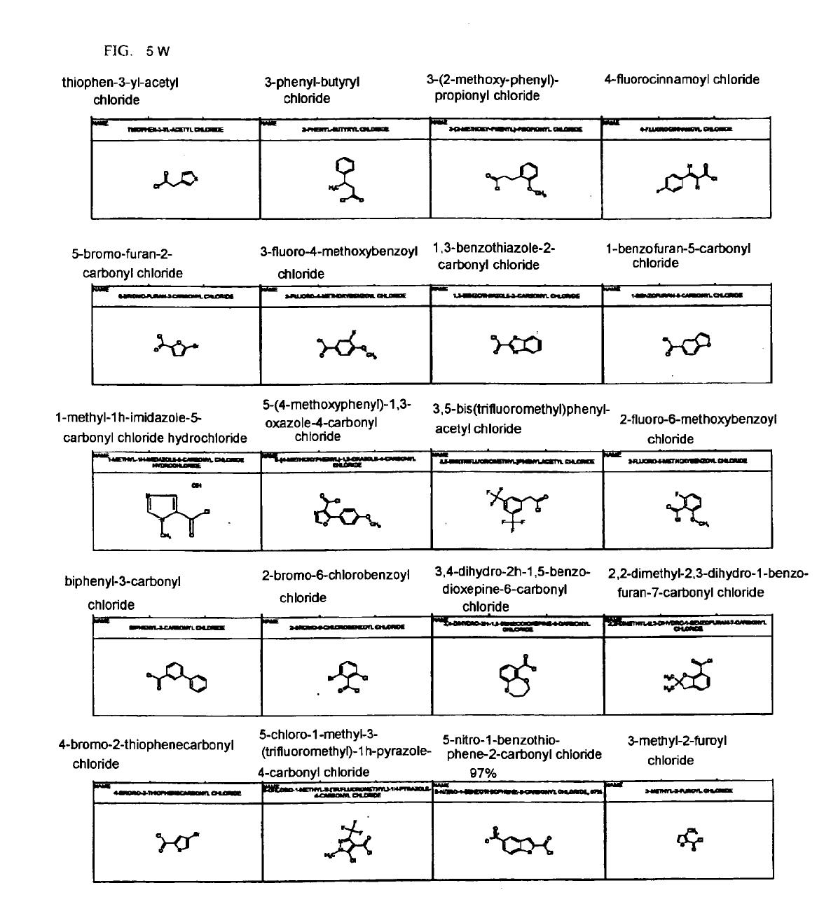

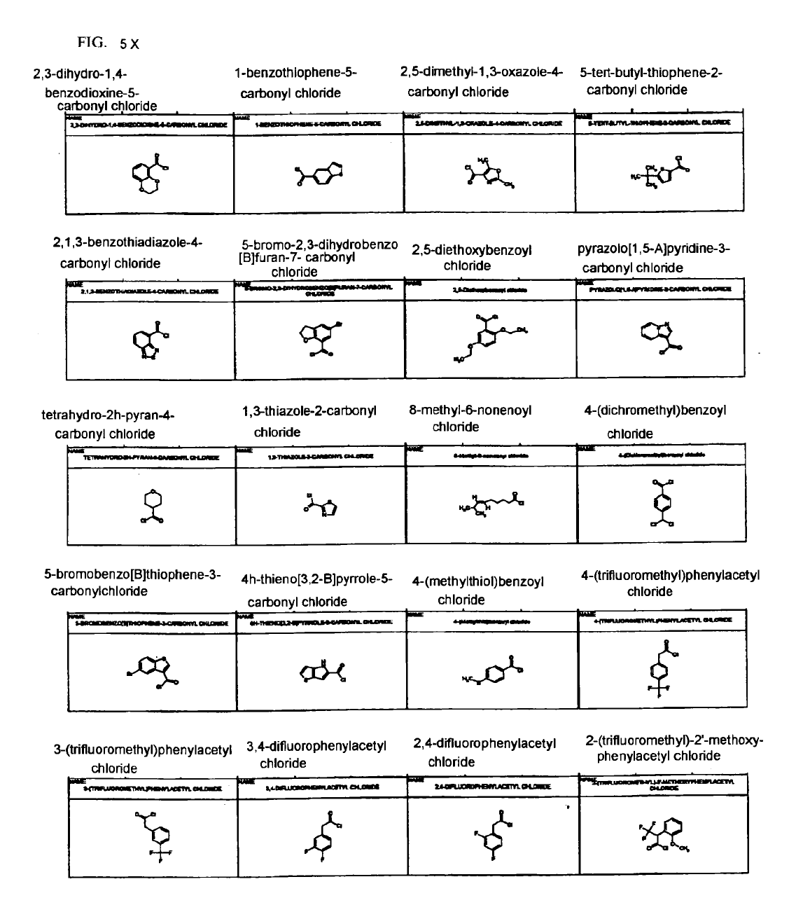

The above-mentioned starting materials are suitable for reacting with a selected acid halide(s) to obtain the imido-substituted 1,4-naphthoquinone compound. Exemplary acid halides are shown in FIG. 5.

In the following description, the imido substitutent may be shown as being ‘symmetrical’ for illustrative purposes and it should be understood that the imido substitutent can be mixed. For example, in a “mixed” imido compound useful in the present methods, the “R” groups in the imido substitutent can be the same or different, and each Y can be the same or different, in which case an “unsymmetrical” or mixed imido substituent is provided.

In the following description, various syntheses and compounds are shown in which X is chloro. It will be appreciated that X is not restricted to chloro. X can be a halogen other than chloro.

In an aspect of the method, Q is represented by:

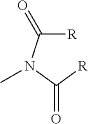

wherein in Q each R is, independently, a substituted or unsubstituted hydrocarbon, provided that one R can, optionally, be hydrogen, and provided that, optionally, R can include at least one hetero atom.

A sub-class of imido-substituted 1,4-naphthoquinones includes those represented by the formula:

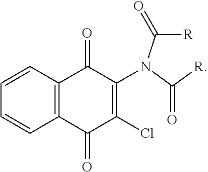

In general, each R is independently a cyclic or acyclic group. Each R includes acyclic, such as straight chain alkyl —(CH₂)ₙCH₃) or branched alkyl, or cyclic, such as cyclo alkyl, or aryl. The expression open-chain imide derivative connotes the case where R is straight or branched alkyl. In another aspect, R can include unsaturation, e.g, an alkenyl. R can be cyclo alkyl, which includes cyclopropyl, cyclobutyl, cyclopentyl, cyclohexyl and the like. By preference, cyclo alkyl is a C₅ to C₇ cyclo alkyl. The R groups may be bonded to each other to form an alkylene bridge, such as a divalent alkylene bridge, although it will be appreciated that the R groups can, together, comprise a polycyclic moiety.

Depending on the R group, the imido compounds can adapt an anti-conformation. For instance, when R is acyclic, the acyclic imido groups can adapt anti-orientation or are capable of some form of staggered orientation, whereas when R is cyclic, the imido groups tend to adapt the syn orientation.

Compounds represented by the foregoing formula can be synthesized by adapting the following representative reaction scheme:

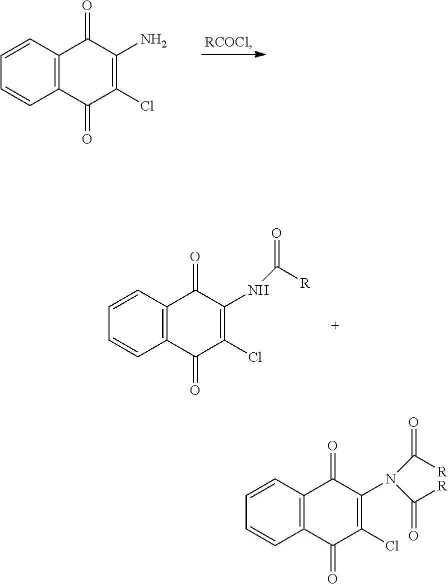

wherein RCOCl is selected to provide the desired R group, and purification of the reaction product yields the intended compound(s) within the general formula for the imido-substituted 1,4-naphthoquinone. Although X is chloro in the example, it will be appreciated that by selecting the appropriate starting material, X can be, for instance, another substituent.

When R is a straight chain alkyl —(CH₂)ₙCH₃, n is generally 0 to 10, preferably 2 to 5, including methyl, ethyl, propyl, butyl, pentyl, and hexyl, such as

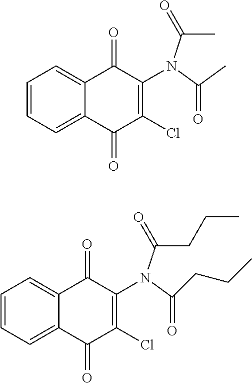 [SMILES: CC(=O)N(C(C)=O)C1=C(Cl)C(=O)c2ccccc2C1=O ; CCCC(=O)N(C(=O)CCC)C1=C(Cl)C(=O)c2ccccc2C1=O]

as examples. Longer R groups are possible. Although X is chloro in the example, it will be appreciated that by selecting the appropriate starting material, X can be, for instance, another substituent.

When R is branched, the branching can be along the chain or can be terminal branching. For terminal branching, an R group can be represented by —(CH₂)ₙCH(CH₃)₂ as an example, where n is from 0 to 6, preferably from 1 to 4, such as

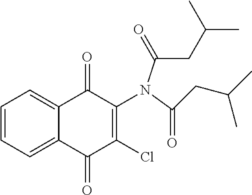 [SMILES: CC(C)CC(=O)N(C(=O)CC(C)C)C1=C(Cl)C(=O)c2ccccc2C1=O]

as an example. Although X is chloro in the example, it will be appreciated that by selecting the appropriate starting material, X can be, for instance, another substituent.

Within the foregoing sub-classes of the above imido-substituted 1,4-naphthoquinones are those in which the open chain imido derivatives have halogen substitution. In one aspect, in a halo-substituted alkylene derivative according to the general formula, the R groups have terminal halo-substitution. Suitable compounds can be synthesized as shown in the following representative exemplary reaction scheme:

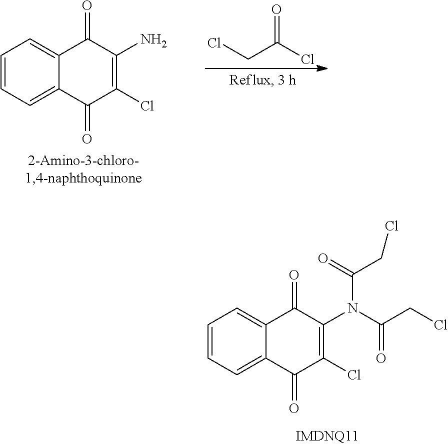

An exemplary chloroacyl chloride reagent is shown for illustrative purposes. Other suitable reagents, such as another acyl dihalide can be selected so that the alkyl group has different halo-substitution, such as a terminally bromo-substituted alkyl group (such as by using bromoacetyl bromide). Mono-halogenation is illustrated but it will be appreciated that other multi-halogenated derivatives are included within the scope of the present methods. Other suitable acyl halides include 2-bromopropionyl chloride, 2-chloropropionyl chloride, 2,3-dibromopropionyl chloride, 2,3-dichloropropionyl chloride, bromoacetyl chloride, 3-bromopropionyl chloride, 4-chloropropionyl chloride, 4-bromopropionyl chloride, 4-bromobutryl chloride, 4-chlorobutryl chloride, 2,4-dibromobutryl chloride, 5-chlorovaleroyl chloride, 5-bromovaleroyl chloride, dichloroacetyl chloride, trichloroacetyl chloride, 6-chloroheanoyl chloride, and the like by examples. Although X is chloro in the example, it will be appreciated that by selecting the appropriate starting material, X can be, for instance, another substituent.

The R groups can also be bonded together to form an alkylene bridge —(CH2)ₙ- in which case n is an integer of 1 to 3, preferably n is 2 or 3, so that Q represents a cyclic imido-substitutent (a nitrogen-containing ring having dione substitution) such as

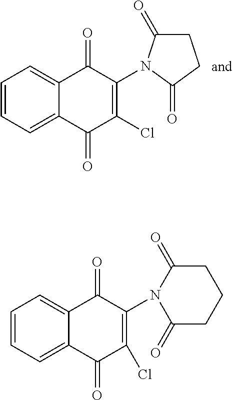 [SMILES: O=C1C(Cl)=C(N2C(=O)CCC2=O)C(=O)c2ccccc21 ; "and" ; O=C1C(Cl)=C(N2C(=O)CCCC2=O)C(=O)c2ccccc21]

to mention examples. The 3-cyclic-imido-substituted 2-halo 1,4-napthoquinone compounds can be synthesized by adapting the following representative reaction scheme:

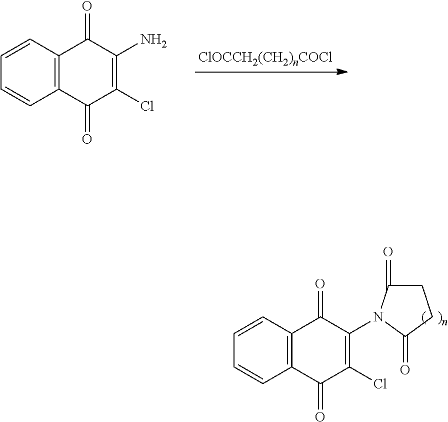

wherein ( ) designates —(CH₂)— and n is an integer of 1 to 3. 2-chloro-3-(N-succinimidyl)-1,4-naphthoquinone is obtained when n is 1. As shown, the succinimidyl derivative (IMDNQ1) has a surprisingly beneficial combination of properties. 2-chloro-3-(N-glutaimidyl)-1,4-napthoquinone is obtained when n is 2. Although X is shown as chloro in the exemplary formulas and in the representative synthesis, it will be appreciated that by selecting the appropriate starting material, X can be, for instance, another substitutent.

A further sub-class of 2-imido-substituted 1,4-naphthoquinones includes derivatives in which the 2-imido-substitution comprises a heterocyclic ring having dione substitution in which the additional hetero atom is preferably oxygen. For instance, the ring can be a five or six member ring with oxygen as an additional hetero atom. An exemplary derivative is a morpholine dione analog, such as IMDNQ14. Morpholine dione analogs can be synthesized as shown in the following exemplary reaction scheme:

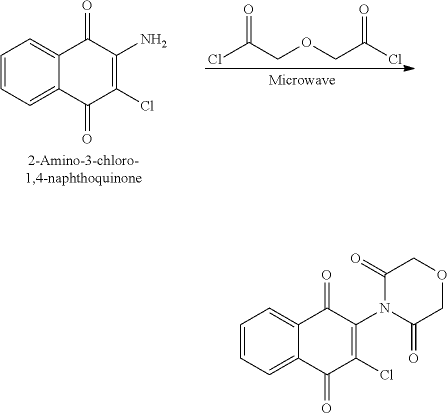 [SMILES: NC1=C(Cl)C(=O)c2ccccc2C1=O ; O=C(Cl)COCC(=O)Cl ; O=C1C(Cl)=C(N2C(=O)COCC2=O)C(=O)c2ccccc21]

X is not restricted to chloro. X can be another halogen, to mention examples. When X is halogen, the microwave treatment can vary in duration and intensity, as seen from Berhe, S., et al., Microwave-assisted synthesis of imido-substituted 2-chloro-1,4-naphthoquinone derivatives and their cytotoxic activities on three human prostate cancer cell lines, Lett. Drug Des. Discov., 5, 485-488 (2008), but typically on lab scale synthesis the duration is on the order of minutes.

A sub-class of imido-substituted 1,4-naphthoquinones includes phthalimidyl derivatives. The compound IMDNQ12 is an example.

A sub-class of imido-substituted 1,4-naphthoquinones includes the cyclic imido-substituted derivatives, which include diarylimido-substituted derivatives. Diarylimido-substituted derivatives, which may be optionally substituted, include those represented by the formula:

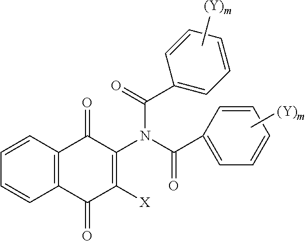

wherein X is hydrogen, halogen, alkoxy (cyclic or alicyclic), trifluoro methyl, aryloxy, or benzyloxy, Y, independently, is hydrogen, halogen, alkoxy, alkyl, halo-alkyl, or halo-alkoxy and m is 0, 1, 2, 3, 4, or 5. When an m=0, Y is hydrogen. X and Y arc independent of each other.

Exemplary diarylimido derivatives having Y halogen substitution include those when m is 1 represented by the formula:

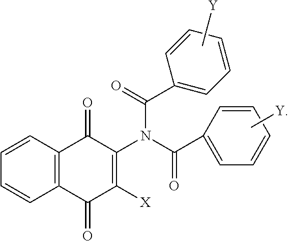

Examples include those compounds denoted herein as IMDNQ1 through IMDNQ6.

Mono-halogen-substituted diarylimido derivatives can be synthesized as shown in the following exemplary reaction scheme:

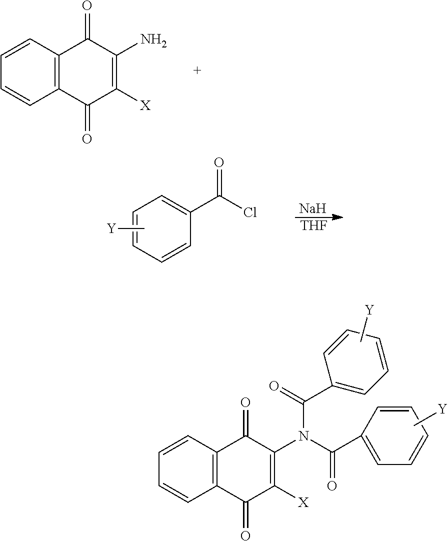

In the exemplary reaction scheme, X is hydrogen, halogen, alkoxy (cyclic or alicyclic), trifluoro methyl, aryloxy, or benzyloxy, and each Y, independently, is H, halogen, alkyl or alkoxy. Co-produced is a mixed imido compound having R hydrogen and the other R is an aryl-imido substituent. X includes bromo, chloro, fluoro, and iodo. Bromo, chloro and fluoro may be preferred when X is halogen. Y may be at the meta, ortho and/or para position of the aryl ring. Meta-halogen substitution on each aryl ring in the imido moiety may be preferred when Y is halogen. Y includes bromo, chloro, fluoro, and iodo. More particularly, Y can be bromo, chloro or fluoro. When Y is a halogen, Y may preferably be chloro or fluoro, with chloro being preferred. IMDNQ4 is a member of this sub-class of imido-substituted 1,4-naphthoquinones. Y can be alkyl, including branched alkyl, or alkoxy as shown in the Examples. When Y is hydrogen, benzoyl chloride can be used.

In general, for the compounds in which an arylimido group is (are) substituted with one or more Y substituent, a synthesis, such as a synthesis above or in the Examples, can be adapted and

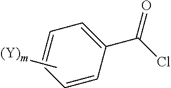

may be used where m is 1, 2, 3, 4 or 5. Acyl halides include 3,5-bis(trifluoromethyl)benzoyl chloride, 2-bromobenzoyl chloride, 2-chlorobenzoyl chloride, 2-fluorobenzoyl chloride, 2-iodobenzoyl chloride, 2-methoxybenzoyl chloride, 2-ethoxybenzoyl chloride, 2-(trifluoromethoxy)benzoyl chloride, 2,4-difluorobenzoyl chloride, 2,6-difluorobenzyol chloride, 2,4-dichlorobenzoyl chloride, 2,6-dichlorobenzoyl chloride, O-acetylsalicyloyl chloride, 2-methoxybenzoyl chloride, 2,6-dimethoxybenzoyl chloride, 2-(trifluoromethyl)benzoyl chloride, 3-bromobenzoyl chloride, 3-chlorobenzoyl chloride, 3-fluorobenzoyl chloride, 3-iodobenzoyl chloride, 3,4-bromobenzoyl chloride, 3,4-di-chlorobenzoyl chloride, 3-methoxybenzoyl chloride, 3,4-dimethoxybenzoyl chloride, 3,4-dimethylbenzoyl chloride, 3,4-difluorobenzoyl chloride, 3,4,5-trimethoxybenzoyl chloride, 3-(trifluoro)benzoyl chloride, 3-(chloromethyl)benzoyl chloride, 4-bromobenzoyl chloride, 4-chlorobenzoyl chloride, 4-fluorobenzoyl chloride, 4-iodobenzoyl chloride, 4-methoxybenzoyl chloride, 4-ethoxybenzoyl chloride, 4-butoxybenzoyl chloride, 4-(hexyloxy)benzoyl chloride, 4-(heptyloxy)benzoyl chloride, 4-(trifluoromethyl)benzoyl chloride, 4-(tert-butyl)benzoyl chloride, 4-(trifluoromethoxy)benzoyl chloride, 4-ethoxybenzoyl chloride, 4-propylbenzoyl chloride, 4-butylbenzoyl chloride, 5-pentylbenzoyl chloride, 4-hexylbenzoyl chloride, 4-heptylbenzoyl chloride, 3,5-dichlorobenzoyl chloride, 2,3-dichlorobenzoyl chloride, 2,3-difluorobenzoyl chloride, 2,5-dichlorobenzoyl chloride, 2,5-difluorobenzoyl chloride, 3,5-dimethoxybenzoyl chloride, 2,4,6-trimethylbenzoyl chloride, 2,4,6,-trichlorobenzoyl chloride, 2,4,6,-trifluorobenzoyl chloride, 2,4,5-trifluorobenzoyl chloride, 2,3,4-trifluorobenzoyl chloride, 2,4-dimethoxybenzoyl chloride, 2,5-dimethoxybenzoyl chloride, 2-fluoro-3-(trifluoromethyl)benzoyl chloride, 2-fluoro-4-(trifluoromethyl)benzoyl chloride, 2-fluoro-5-(trifluoromethyl)benzoyl chloride, 3-fluoro-5-(trifluoromethyl)benzoyl chloride, 4-fluoro-2-(trifluoromethyl)benzoyl chloride, 4-fluoro-3-(trifluoromethyl)benzoyl chloride, 5-fluoro-2-(trifluoromethyl)benzoyl chloride, 2-fluoro-6-(trifluoromethyl)benzoyl chloride, 2,4-bis(trifluoromethyl)benzoyl chloride, 2,6-bis(trifluoromethyl)benzoyl chloride, 3-(tri fluoromethoxy)benzoyl chloride, 2,3,4,5-fluorobenzoyl chloride, 2,4-dichloro-5-fluorobenzoyl chloride, 3-(dichloromethyl)benzoyl chloride, 2,3,5-trifluorobenzoyl chloride, 3,4,5-trifluorobenzoyl chloride, 2-chloro-6-fluorobenzoyl chloride, 3-chloro-4-fluorobenzoyl chloride, 4-chloro-2,5-difluorobenzoyl chloride, 5-fluoro-2-methylbenzoyl chloride, 3-fluoro-4-methylbenzoyl chloride, 2,6-difluoro-3-methylbenzoyl chloride, 3-chloro-2-6-(trifluoromethyl)benzoyl chloride, 5-chloro-2-(trifluoromethyl)benzoyl chloride, and 2-chloro-6-fluoro-3-methylbenzoyl chloride, 6-chloro-2fluoro-3-methylbenzoyl chloride, 2-chloro-5-fluorobenzoyl chloride, 4-fluoro-3-methyl chloride, 5-chloro-2-fluorobenzoyl chloride, 2-chloro-3,6-fluorobenzoyl chloride, 3-chloro-2,4-fluorobenzoyl chloride, 3-chloro-2-fluoro-5(trifluoromethyl)benzoyl chloride, 4-methoxy-3-(trifluromethyl)benzoyl chloride, 4-methyl3-(trifluoromethyl)benzoyl chloride, 2-chloro-5-(trifluoromethyl)benzoyl chloride, 2,3-difluoro-4-methylbenzoyl chloride, 3,5-dichloro-4-methoxybenzoyl chloride, 2,4,5-trifluoro-3-methoxybenzoyl chloride, 2,3,4,6-tetrafluorobenzoyl chloride, 5-bromo-2,3,4-trimethylbenzoyl chloride, 4-bromo-2,6-difluorobenzoyl chloride, 2-fluoro-5-iodobenzoyl chloride, 2-fluoro-6-iodobenzoyl chloride, 4-bromo-2-fluorobenzoyl chloride, and 2-bromo-6-chlorobenzoyl chloride by way of example.

Other imido-substituted 1,4-naphthoquinones with different Q moieties can be obtained with other acid halides, including those disclosed in FIG. 5, such as, for example, O-acetylmandelic chloride, phenoxyacetyl chloride, 4-chlorophenoxyacetyl chloride, phenylacetyl chloride, cinnamoyl chloride, hydrocinnamoyl chloride, 2-chloro-2,2diphenylacetyl chloride, alpha-chlorophenylacetyl chloride, 1-napthoyl chloride, 2-napthoyl chloride, 3,4(dimethoxy)benzoylacetyl chloride, 3-methoxyphenylacetyl chloride, 3-phenoxyproprionyl chloride, 2-(1-naphthyl)ethanoyl chloride, and 2-(3,5-difluorophenyl)ethanoyl chloride, 2-bromophenylacetyl chloride, 3-acetoxy-2-methylbenzoyl chloride, by way of alternative aryl groups.

It will be appreciated that when an R group is aryl or aryloxy or cyclo alkyl (which includes polycyclic alkyl), there may be an intervening linking group (sometimes called a spacer group) between the aryl or aryloxy or cyclo alkyl group to the imido-functional group. An exemplary such linking group would be an alkylene group, as an example.

In each of the various aspects of the present inventions, a Y substituent can be substituted alkyl, such as halogen-substituted alkyl, including trifluoro methyl, or substituted alkoxy, such as halogen-substituted alkoxy, including trifluoromethoxy, to mention examples.

The imido-substituted 1,4-naphthoquinone compound can be symmetrical or mixed, such as shown in the Examples. An exemplary reaction scheme for preparing a sub-class of imido-substituted 1,4-naphthoquinones having mixed Y group(s) can be represented as follows:

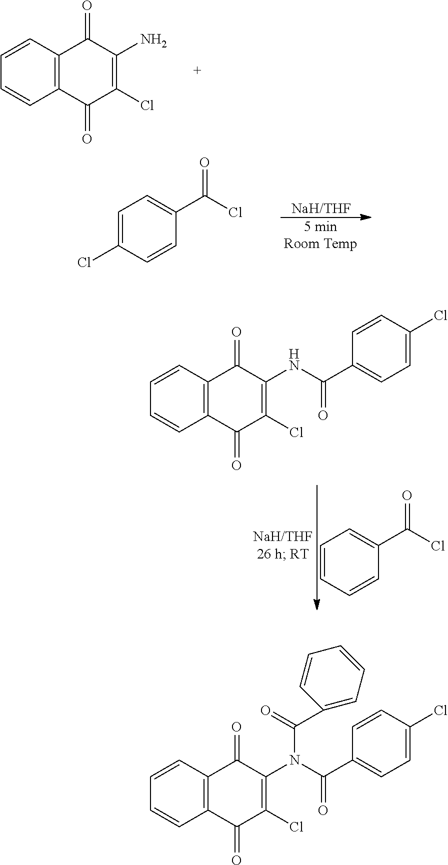

It will be appreciated that an unsymmetrical imido substituent, e.g., “mixed” as to an aryl ring(s), the Y substituent(s), and/or in the position(s) of a Y substituent(s) may be achieved by selecting a desired member from the class of acid chlorides from the class of benzoyl chlorides for the first step, and a different member for the second step. It will also be appreciated that a “mixed” imido-substituted 1,4-naphthoquinone is obtained in the first step wherein, for instance, the imido-nitrogen is bonded to hydrogen (one of the R groups) and the other R is substituted aryl. Other “mixed” compounds are obtained by adapting an appropriate synthesis and using an appropriate acid halide, which includes the exemplary acid chlorides in FIG. 5. For instance, other “mixed” imido-substituted 1,4-naphthoquinione compounds include an R being other than hydrogen and the other R being a different substituted or unsubstituted hydrocarbon.

Another sub-class of imido-substituted 1,4 includes naphthoquinones unsymmetrical alkyl aryl imido-substituted naphthoquinones which can be synthesized by adapting the following representative reaction scheme. An aminonaphthoquinone analog is first converted to the alkyl amido derivative which is subsequently reacted with an aryl acid chloride in the presence of an alkalin hydride, such as sodium hydride, in anhydrous THF to furnish the unsymmetrical alkyl aryl imidonaphthoquinone derivative.

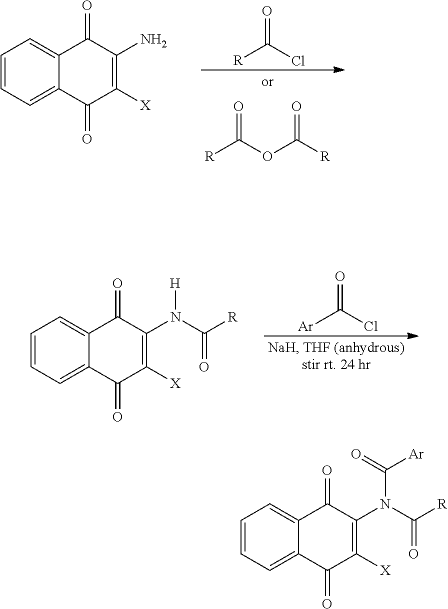

An R group can be alkyl, such as described elsewhere herein, which includes C₁-C₆ alkyl. The other R group can be an aromatic group, such as an aryl group, such as described elsewhere herein. X can be a substitutent as described elsewhere herein, which includes, for example, hydrogen, halogen, alkyl, alkoxy (such as lower alkoxy, methoxy or the like).

A 1,4-naphthoquinone starting material as shown in various reaction schemes herein is chloro substituted (X=chloro) only for illustrative purposes. It will be appreciated that the 1,4-naphthoquinone starting material can have a 3-substitution so that X in the general formula and in the various formulas can be other than chloro.

A difference in anti-Leishmanial activity against Leishmania donovani promastigotes vs. amastigotes is observed. The differential susceptibility determines which in vitro models are appropriate for either drug screening or resistance monitoring of clinical field isolates.

The ratio between the toxic dose and the therapeutic dose of a drug is a selectively index. It is used as a measure of the relative safety of the drug for a particular treatment. The selectivity index (SI) herein is the ratio of IC₅₀ for fibroblast cells/IC₅₀ for parasites and was calculated to compare the toxicity for mammalian cells and the activity against Leishmania donovani. The presently prescribed Amphotericin B has a selectivity index of 0.05 in amastigotes and 0.22 in promastigotes. In one aspect of the method, the selectivity index of all compounds used except IMDNQ5 (in promastigotes) is greater than the selectivity index for Amphotericin B in promastigotes and amastigotes. Further, in an aspect of the invention, some representative compounds are relatively non-cytotoxic to Balb/C 3T3 mouse fibroblast cell line with IC₅₀ values of well below the value compared to Amphotericin B.

The in vitro testing shows the present method should have in vivo efficacy in inhibiting proliferation of Leishmania donovani, and thus indicating a disease caused by the Leishmania genus may be treated by administering a compound according to the general formula.

Inhibitory concentration is typically evaluated at the 50% inhibitory concentration (IC₅₀). Inhibition of proliferation may be attained at a lower concentration in practice, but an IC₅₀ concentration may be desirable.

Representative imido-substituted 1,4-napthoquinone compounds, and their synthesis, are described in Bakare, O., et al, Synthesis and MEK1 inhibitory activities of imido-substituted 3-chloro-1,4-naphthoquinones. Bioorg. Med. Chem., 11, 3165-3170 (2003); Berhe, S., et al., Microwave-assisted synthesis of imido-substituted 3-chloro-1,4-naphthoquinone derivatives and their cytotoxic activities on three human prostate cancer cell lines, Lett. Drug Des. Discov., 5, 485-488 (2008); Akinboye et al., Acta Cryst. E65, o24 (2009), and Akinboye et al., Acta Cryst. E65, o277 (2009), the complete disclosures of which are incorporated herein by reference.

A patient in need of treatment may be diagnosed by testing and by physical examination. Testing includes serological tests, immunoassays and PCR methods to diagnose for the presence of Leishmania parasites infection in an individual. The testing is sometimes performed in tandem. Serological testing of blood samples from an individual can yield negative and positive sero results. A so-called sero-positive result is indicative of infection. So-called sero-negative results may or may not indicate the absence of infection. The primary limitation of this technique revolves around interpretation of a positive titer which may only indicate exposure to the parasite as opposed to active infection. However, due to the disease progression more than a single test with a single sero-negative result is preferred. PCR methods can be used in determining a patient in need of treatment. The most reliable diagnostic test relies on demonstration of Leishmania parasites either cytologically or histopathologically, in stained preparations of bone marrow, lymph node, spleen, skin or other tissues and organs (skeletal muscle, peripheral nerves, renal interstitium, and synovial membranes. Leishmania parasites most commonly reside in macrophages, but have been observed in other cell lines including neutrophils, eosinophilis, endothelial cells and fibroblasts. While microscopic visualization of parasites provide a definitive diagnosis, this technique may be only 60% effective for bone marrow samples and 30% effective for lymph node specimens, making it less sensitive than other testing strategies.

Diagnosis of a patient in the acute phase of leishmaniasis disease who is in need of treatment may include physical examination. The acute stage may extend for a few weeks or months following initial infection. Many symptoms may not be unique to leishmaniasis disease.

The methods described herein advantageously utilize an active ingredient such as 2-imido-substituted 3-halo-1,4-naphthoquinones, as a novel class of selective anti-Leishmanial agents effective against Leishmania parasites.

The expression imido-substituted 1,4-naphthoquinone includes a compound according to the general formula.

The complete disclosure of each reference cited herein is incorporated by reference.

Those skilled in the art will recognize that modifications and variations may be made without departing from the true spirit and scope of the invention. The invention, therefore, is not to be limited to the embodiments described and illustrated in the following non-limiting examples but is to be determined from the appended claims.

The following non-limiting Examples illustrate the invention without limiting its scope.

### EXAMPLES

In the Examples, reactions were carried out using laboratory grade materials and solvents. Melting points were determined in open capillary tubes on a Mel-Temp melting point apparatus and are uncorrected. The IR spectra were recorded on a Perkin Elmer PE 100 spectrometer with an Attenuated Total Reflectance (ATR) window. The and ¹³C-NMR spectra were obtained on a Bruker Advance 400 MHz spectrometer in deuterated chloroform (CDCl₃). Chemical shifts are in δ units (ppm) with TMS (0.00 ppm) or CHCl₃ (7.26 ppm), as internal standard for ¹H-NMR, and CDCl₃ (77.00 ppm) for ¹³C-NMR. Electrospray ionization mass spectrometry was recorded on a Thermo LTQ Orbitrap XL mass spectrometer and compounds dissolved in acetonitrile with 0.1% formic acid. The known intermediates were prepared according to procedures that are reported in the literature.

Exemplary imido-substituted naphthoquinone compounds IMDNQ 1 through IMDNQ15 were prepared in the Examples.

### Examples 1 through 6

A general procedure for the synthesis of aryl-imido-substituted 1,4-naphthoquinones represented by the formula

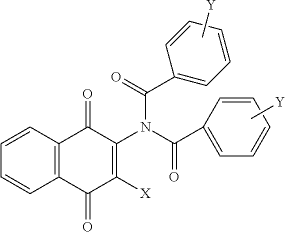

wherein X is hydrogen, halogen, alkoxy (cyclic or alicyclic), trifluoro methyl, aryloxy, or benzyloxy and Y is halogen is exemplified with respect to IMDNQ1-IMDNQ6, respectively in Examples 1 through 6. 2-Amino-3-chloro-1,4-naphthoquinone (1.47 mmol) or the 3-bromo-analog was dissolved in THF (15 mL). NaH (3.08 mmol) was added and the mixture stirred at room temperature for 15 min. Appropriate acid chloride (3.08 mmol) was added drop wise, and the resulting mixture stirred at room temperature for 24 hours. The THF was then evaporated under vacuum and ice-cooled water added to the residual mixture. The resulting aqueous mixture was extracted with CH₂Cl₂ (2×30 mL) and the combined organic phase washed with water (3×15 mL), saturated NaCl solution (15 mL) and dried over anhydrous MgSO₄. The crude product was purified by triturating in hot ethanol followed by recrystallization in ethyl acetate and/or column chromatography on silica gel.

### Example 1

### 4-Chloro-N-(4-chlorobenzoyl)-N-(3-chloro-1,4-dioxo-1,4-dihydronaphthalen-2-yl)-benzamide (IMDNQ 1)

Yellow solid. (27%). Mp 212-213° C. IR (cm⁻¹) 1737.38, 1714.75, 1696.91, 1673.62, 1589.20, 1571.10. ¹H NMR (CDCl₃) 7.33-7.36 (m, 4H), 7.68-7.72 (m, 4H), 7.79-7.85 (m, 2H), 8 09-8.11 (m, 1H), 8.20-8.22 (m, 1H). ¹³C NMR (CDCl₃) 127.72, 127.80, 129.18, 130.34, 130.50, 131.32, 132.52, 134.92, 134.95, 139.67, 142.46, 143.87, 170.40, 177.05, 178.59. ESI MS m/z 505.975 ([M+Na]⁺ calcd 505.973).

### Example 2

### 2-Chloro-N-(2-chlorobenzoyl)-N-(3-chloro-1,4-dioxo-1,4-dihydronaphthalen-2-yl)-benzamide (IMDNQ 2)

Yellow solid. (49%). Mp 217-218° C. IR (cm⁻¹) 1720.19, 1681.22, 1618.00, 1588.02. ¹H NMR (CDCl₃) 7.10-7.16 (m, 2H), 7.21-7.31 (m, 4H), 7.79-7.89 (m, 4H), 8.17-8.20 (m, 1H), 8.20-8.26 (m, 1H). ¹³C NMR (CDCl₃) 126.76, 126.93, 127.60, 127.80, 129.69, 130.54, 130.85, 131.37, 132.36, 132.52, 132.56, 132.75, 134.14, 134.75, 134.91, 142.65, 144.43, 177.13, 178.22. ESI MS m/z 505.9741 ([M+Na]⁺ calcd 505.9730).

### Example 3

### N-(3-Chloro-1,4-dioxo-1,4-dihydronaphthalen-2-yl)-4-fluoro-N-(4-fluorobenzoyl)-benzamide (IMDNQ 3)

Yellow solid. (56%). Mp 284-286° C. IR (cm⁻¹) 3074.94, 1719.61, 1689.97, 1672.75, 1591.10. ¹H NMR (CDCl₃) 7.00-7.06 (m, 1H), 7.75-7.85 (m, 1H), 8.10-8.12 (m, 6H), 8.20-8.22 (m, 4H). ¹³C NMR (CDCl₃) 115.97, 116.19, 116.41, 126.65, 126.96, 127.67, 127.77, 130.53, 130.57, 130.58, 131.35, 131.62, 131.71, 132.75, 134.88, 142.42, 144.13, 164.14, 166.68, 170.33, 177.13, 178.63. ESI MS m/z 474.034 ([M+Na]⁺ calcd 474.032).

### Example 4

### 3-Chloro-N-(3-chlorobenzoyl)-N-(3-chloro-1,4-dioxo-1,4-dihydronaphthalen-2-yl)-benzamide (IMDNQ 4)

Yellow solid. (31%). Mp 258-260° C. IR (cm⁻¹) 3075.36, 1713.64, 1698.11, 1672.50, 1591.48, 1571.31. ¹H NMR (CDCl₃) 7.27-7.31 (t, J=7.85 Hz, 2H), 7.42-7.45 (ddd, J=1.03, 2.09, 8.07 Hz, 2H), 7.60-7.63 (td, J=1.07, 7.68 Hz, 2H), 7.70-7.71 (t, J=1.82, 2H), 7.80-7.85 (m, 2H), 8.11-8.14 (m, 1H), 8.21-8.23 (m, 1H). ¹³C NMR (CDCl₃) 127.02, 127.93, 128.03, 129.34, 130.21, 130.75, 131.55, 133.29, 135.14, 135.21, 136.06, 143.00, 143.84, 170.20, 177.25, 178.66. ESI MS m/z 505.9741 ([M+Na]⁺ calcd 505.9730).

### Example 5

### N-(3-Bromo-1,4-dioxo-1,4-dihydronaphthalen-2-yl)-2-chloro-N-(2-chlorobenzoyl)-benzamide (IMDNQ 5)

Yellow crystal (34%). Mp 232-234° C. IR (cm⁻¹) 1728.78, 1688.76, 1671.10, 1588.22, 1468.70. ¹H NMR (CDCl₃) 7.13 (2H, J=7.9 Hz), 7.21 (2H, J=1.6, 7.9 Hz), 7.28 (dd, 2H, J=1.3, 7.5 Hz), 7.78-7.86 (m, 2H), 7.94 (d, 2H, 5.2 Hz), 8.15-8.21 (m, 1H), 8.22-8.28 (m, 2H). ¹³C NMR (CDCl₃) 126.65, 127.66, 128.08, 129.63, 130.49, 130.81, 131.23, 132.25, 134.68, 134.80, 140.78, 145.81, 167.57, 177.26, 177.81. ESI MS m/z 549.9240 ([M+Na]⁺ calcd 549.9224).

### Example 6

### P N-(3-Bromo-1,4-dioxo-1,4-dihydronaphthalen-2-yl)-4-fluoro-N-(4-fluorobenzoyl)-benzamide (IMDNQ 6)

Yellow solid (66%). Mp: 170-172° C. IR (cm⁻¹) 1719.23, 1670.56, 1596.69, 1505.57. ¹H NMR (CDCl₃) 7.05 (t, 4H, J=12.0 Hz), 7.77-7.88 (m, 6H), 8.08-8.15 (m, 1H), 8.21-8.27 (m, 1H). ¹³C NMR (CDCl₃) 115.57, 115.79, 127.4, 127.71, 130.18, 130.30, 130.33, 130.85, 131.35, 131.44, 134.46, 134.50, 138.52, 146.90, 165.03, 169.91, 176.96, 177.97. ESI MS m/z 517.9785 ([M+Na]⁺ calcd 517.9815).

### Example 7

2-Amino-3-chloro-1,4-naphthoquinone (IMD7) was prepared by refluxing commercially available 2,3-dichloro-1,4-naphthoquinone with ammonia/ammonium hydroxide mixture in ethanol.

### Example 8

2-Amino-3-bromo-1,4-naphthoquinone (IMD8) was prepared by refluxing commercially available 2,3-dibromo-1,4-naphthoquinone with ammonia/ammonium hydroxide mixture in ethanol.

### Example 9

2-amino-3-chloro-1,4-naphthoquinone was dissolved in THF (15 mL). NaH was added and the mixture was stirred at room temperature for 15 mins. The 4-methoxybenzoyl chloride was added, drop wise, and the mixture was stirred for 24 hours. (Mole ratio of Substrate:NaH:Acid Chloride (1:2.3:2.3)) THF was evaporated under vacuum and the mixture was washed with ice-water (10 g ice and 10 mL water). The ice-water mixture was extracted with CH₂Cl₂ (30 mL, 20 mL consecutively) and the combined organic phase washed with water (3×20 mL), saturated NaCl solution (3×20 mL), then dried over anhydrous MgSO₄. The crude was purified via triturating in hot ethanol, recrystallization in ethyl acetate and/or via column chromatography.

### 2-bis-(4-methoxybenzoyl)amino-3-chloro-1,4-naphthoquinone: (IMDNQ 9)

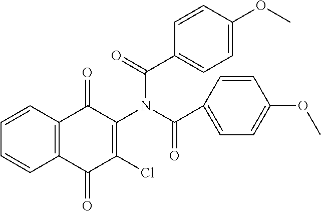 [SMILES: COc1ccc(C(=O)N(C(=O)c2ccc(OC)cc2)C2=C(Cl)C(=O)c3ccccc3C2=O)cc1]

Obtain a yellow solid. (47.9%). Mp 283-287° C. IR (cm⁻¹) 3019.56, 1698.7, 1668.81, 1599.28, 1574.26, 1508.65. ¹H NMR (CDCl₃). 3.81 (s, 6H), 6.80-6.84 (td, J=2.86, 8.95 Hz, 4H), 7.73-7.82 (m, 6H), 8.09-8.11 (m, 1H), 8.19-8.21 (m, 1H). ¹³C NMR (CDCl₃) 55.48, 113.98, 114.21, 126.76, 127.57, 127.64, 130.38, 130.79, 131.41, 131.48, 134.61, 134.66, 141.69, 144.95, 163.34, 171.03, 177.48, 178.77.

### Example 10

IMDNQ10 derivative was synthesized from 2-amino-3-chloro-1,4-naphthoquinone and the appropriate acid chloride in accordance with Bakare, O., et al, Synthesis and MEK1 inhibitory activities of imido-substituted 2-chloro-1,4-naphthoquinones. Bioorg. Med. Chem., 11, 3165-3170 (2003); and Berhe, S., et al., Microwave-assisted synthesis of imido-substituted 2-chloro-1,4-naphthoquinone derivatives and their cytotoxic activities on three human prostate cancer cell lines, Lett. Drug Des. Discov., 5, 485-488 (2008).

### Example 11

2-amino-3-chloro-1,4-naphthoquinone was dissolved in THF (15 mL). NaH was added and the mixture was stirred at room temperature for 15 mins. The 3,4,5-(trimethoxy)benzoyl chloride was added, drop wise, and the mixture was stirred for 24 hours. (Mole ratio of Substrate:NaH:Acid Chloride (1:2.3:2.3)) THF was evaporated under vacuum and the mixture was washed with ice-water (10 g ice and 10 mL water). The ice-water mixture was extracted with CH₂Cl₂ (30 mL, 20 mL consecutively) and the combined organic phase washed with water (3×20 mL), saturated NaCl solution (3×20 mL), then dried over anhydrous MgSO₄. The crude was purified via triturating in hot ethanol, recrystallization in ethyl acetate and/or via column chromatography. 2-bis-(3,4,5-trimethoxybenzoyl)amino-3-chloro-1,4-naphthoquinone: (IMDNQ 11)

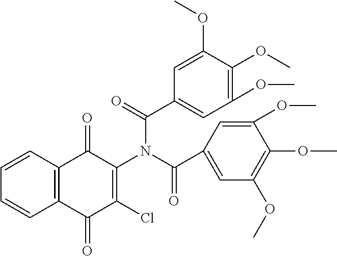 [SMILES: COc1cc(C(=O)N(C(=O)c2cc(OC)c(OC)c(OC)c2)C2=C(Cl)C(=O)c3ccccc3C2=O)cc(OC)c1OC]

Obtain orange crystals (51.6%). Mp 171-172° C. IR (cm⁻¹) 3015.37, 2939.81, 2838.40, 1704.21, 1675.26, 1586.02, 1122.75. ¹H NMR (CDCl₃) 3.81 (s, 12H), 3.84 (s, 6H), 7.02 (s, 4H), 7.81-7.84 (m, 2H), 8.12-8.14 (m, 1H), 8.22-8.24 (m, 1H). ¹³C NMR (CDCl₃) 56.27, 56.35, 60.93, 127.62, 127.77, 129.31, 130.69, 131.39, 134.86, 142.06, 142.27, 144.45, 153.01, 171.08, 177.27, 178.84.

### Example 12

The phthalimidyl (IMDNQ12) derivative was synthesized from 2-amino-3-chloro-1,4-naphthoquinone and the appropriate acid chloride in accordance with Bakare, O., et al, Synthesis and MEK1 inhibitory activities of imido-substituted 2-chloro-1,4-naphthoquinones. Bioorg. Med. Chem., 11, 3165-3170 (2003); and Berhe, S., et al., Microwave-assisted synthesis of imido-substituted 2-chloro-1,4-naphthoquinone derivatives and their cytotoxic activities on three human prostate cancer cell lines, Lett. Drug Des. Discov., 5, 485-488 (2008).

### Example 13

The mono-butryl derivative (IMDNQ13) was synthesized from 2-amino-3-chloro-1,4-naphthoquinone and the appropriate acid chloride in accordance with Bakare, O., et al, Synthesis and MEK1 inhibitory activities of imido-substituted 2-chloro-1,4-naphthoquinones. Bioorg. Med. Chem., 11, 3165-3170 (2003); and Berhe, S., et al., Microwave-assisted synthesis of imido-substituted 2-chloro-1,4-naphthoquinone derivatives and their cytotoxic activities on three human prostate cancer cell lines, Lett. Drug Des. Discov., 5, 485-488 (2008).

### Example 14

The morpholine dione analog (IMDNQ14) was synthesized by microwave irradiation of a mixture of 2-amino-3-chloro-1,4-naphthoquinone and diglycolyl chloride as depicted in scheme 1 in Berhe, S., et al., Microwave-assisted synthesis of imido-substituted 2-chloro-1,4-naphthoquinone derivatives and their cytotoxic activities on three human prostate cancer cell lines, Lett. Drug Des. Discov., 5, 485-488 (2008).

### Example 15

The dibutryl (IMDNQ15) derivative was synthesized from 2-amino-3-chloro-1,4-naphthoquinone and the appropriate acid chloride in accordance with Bakare, O., et al, Synthesis and MEK1 inhibitory activities of imido-substituted 2-chloro-1,4-naphthoquinones. Bioorg. Med. Chem., 11, 3165-3170 (2003); and Berhe, S., et al., Microwave-assisted synthesis of imido-substituted 2-chloro-1,4-naphthoquinone derivatives and their cytotoxic activities on three human prostate cancer cell lines, Lett. Drug Des. Discov., 5, 485-488 (2008).

Activity and Potency of 15 Compounds Against Leishmania donovani Parasites

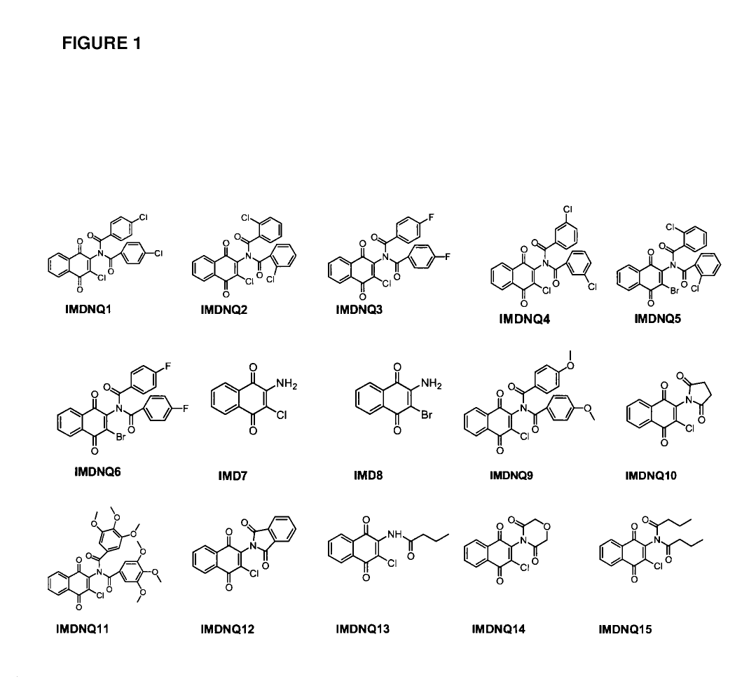

The activity and/or potency of naphthoquinione compounds (FIG. 1) was evaluated against Leishmania donovani parasites.

The 15 compounds were screened using both promastigote and amastigote forms of Leishmania donovani parasites. A cut off of at least 50% growth inhibition in the screening was an initial screening criteria to make the evaluation more facile. Six active compounds met this initial screening criteria. All 15 compounds were screened using the Resazurin assay. Low levels of cellular toxicity and selectivity indices of a compound were factors in screening that led to the six compounds. IC₅₀ values were different in promastigotes and amastigotes but had a similar pattern in most of the screened compounds. Amphotericin B had an IC₅₀ value of 5.26 μM in promastigotes and 22.26 μM in amastigotes, while IMDNQ2, IMDNQ3, IMDNQ4, IMDNQ5, IMD8 and IMDNQ15 had IC₅₀ values of 2.27 μM, 10.81 μM, 6.81 μM, 31.28 μM, 4.3 μM, and 0.05 μM in promastigotes and 5.83 μM, 4.10 μM, 1.19 μM, 4.67 μM, 2.07 μM, 18.85 μM in amastigotes, respectively. The six screened compounds had very low cytotoxicity in mice fibroblast cells compared with the standard drug, Amphotericin B. The IC₅₀ values of these compounds in mice fibroblasts cells were much higher (78.75 μM, 24.83 μM, 168.1 μM, 4.7 μM, 14197.35 μM, and 2.94 μM) compared to that of Amphotericin B which was 1.18 μM. Selected candidate compounds have demonstrated high leishmanicidal activities on either promastigotes, amastigotes or on both forms of the parasites and have proven to have acceptable toxicity at limited dose.

The naphthoquinone compounds used in the current disclosure have limited cytotoxicity levels to human cancer cell lines and confirmed in mice fibroblast cells. Fibroblast cells have been documented as natural host cells in Latent leishmaniasis (Bogdan ct al. J. Expermental Medicine Vol 191 (12), pp 2121-2130, 2000).

the Present Naphthoquinone Compounds have Low Cytotoxicity on Mice Fibroblast Cells

Fibroblasts cells (4×10⁴ cells/nil and adding 100 μl per well) were incubated with different concentrations of the naphthoquinone compounds (FIGS. 1) (3, 7, 13, 29, 33, and 44 μM) for 48 hrs after which viability of live fibroblast cells was determined by the Resazurin Method using a microplate reader (Bio-Tek Instruments EL311). The IC₅₀ values for the compounds were calculated and ranged from 2.94 for the lowest to 14197.35 for the highest. All compounds had higher IC₅₀ values compared to Amphotericin B. (1.18 μM). This showed that all compounds (IMDNQ1-IMDNQ15) are less toxic than the standard drug Amphotericin B. All compound stocks were dissolved in 100% DMSO and all working concentrations were made by diluting compounds in distilled water (1% DMSO). IMD7, IMD8, IMDNQ4, IMDNQ2, and IMDNQ3 exhibited very low cytotoxicity, with IC₅₀ values being 1448.56 μM, 14197.35 μM, 168.1 μM, 78.75 μM, and 24.83 μM against mouse fibroblasts cells.

### In Vitro Anti-Leishmanial Activities Against Leishmania Donovani Promastigotes and Amastigotes.

In vitro anti-Leishmanial activities of the compounds (FIG. 1) were evaluated using axenic Leishmania donovani promastigotes and amastigotes using the Rezasurin Viability Assay. In all 15 compounds screened, IC₅₀ values were compared to Amphotericin B. Values for compounds 1, 2, 7 and 8 were lower than that for Am. B. while all other compounds had higher IC50 values compared to Am. B. Selectivity indices of compounds were also mostly higher than that of Am. B. Only IMDNQ5 (0.15 μM) and IMDNQ15 (0.048 nM) had lower selectivity values compared to Am. B. Compounds IMNDQ1 (2.28 μM), IMDNQ2 (34.69 μM), IMDNQ3 (2.30 μM), IMDNQ4 (24.65 μM), IMDNQ6 (0.44 μM), IMD7 (331.48 μM), IMD8 (3301.71 μM), IMDNQ9 (0.60 μM), IMDNQ10 (0.69 μM), IMDNQ11 (0.28 μM) and IMDNQ12 (0.91 μM), IMDNQ13 (0.62 μM), IMDNQ14 (0.40 μM), had higher selectivity indices than Am.B. Therefore, all the compounds except IMDNQ5 and IMDNQ15 are more selective than Am. B. anti-Leishmanial activities of compounds against Leishmania donovani amastigotes showed variable IC₅₀s compared with Am. B. (5.98 μM). Values ranged from 1.19 μM for IMDNQ4 to 27.31 μM for IMDNQ11. All compounds, except IMDNQ11 had lower 10₅₀ values in Leishmania donovani amastigotes compared to the standard drug Amphotericin B.

### Selectivity Indices Against Leishmania Donovani Promastigotes and Amastigotes.

Naphthoquinone compounds (FIG. 1) used are generally more selective against Leishmania donovani parasites compared to the standard drug, Amphotericin B. In amastigotes forms of the parasites, Amphotericin B. had a selectivity index of 0.05 while all the screened compounds had selectivities ranging from 0.16 to 6858.62 as indicated in Table 1. In promastigotes, Amphotericin B. had a selectivity index of 0.22 much lower than all compounds used in the study except IMDNQ5 whose selectivity index was 0.15 (Table 1).

The exposure to the compounds in relation to time was done to be able to evaluate the effect of time on each of the compounds. Exposure of promastigotes to the compounds was done for 2 hrs, 4 hrs, 6 hrs, and 24 hrs. Exposure time that had a significant effect was at 24 hrs of exposure.

Table 1 below indicates the tabulated anti-Leishmanial activities in promastigotes and amastigotes of Leishmania donovani, cytotoxicities in mice fibroblast cells, and the selectivity indices of the listed compounds.

TABLE 1
In Vitro Activity of Fifteen Naphthoquinone Compounds and Amphotericin
B against Leishmania. donovani promastigotes and amastigotes.

| Tested | IC [symbol] | | | SI Values | |
|---|---|---|---|---|---|
| Compound | Promastigotes | Amastigotes | Fibroblasts | Amastigotes | Promastigotes |
| IMDNQ1 | 1.84 | 11.85 | 4.1 | 0.35 | 2.23 |
| IMDNQ2 | 2.27 | 5.83 | 78.75 | 13.51 | 34.69 |
| IMDNQ3 | 10.81 | 4.10 | 24.83 | 6.06 | 2.30 |
| IMDNQ4 | 6.82 | 1.19 | 168.1 | 141.26 | 24.65 |
| IMDNQ5 | 31.28 | 4.67 | 4.7 | 1.01 | 0.15 |
| IMDNQ6 | 9.66 | 9.32 | 4.25 | 0.46 | 0.44 |
| IMD7 | 4.37 | 12.28 | 1448.56 | 117.96 | 331.48 |
| IMD8 | 4.3 | 2.07 | 14197.35 | 6858.62 | 3301.71 |
| IMDNQ9 | 5.64 | 4.67 | 3.41 | 0.73 | 0.60 |
| IMDNQ10 | 7.0 | 1.69 | 4.83 | 2.86 | 0.69 |
| IMDNQ11 | 22.9 | 27.31 | 6.41 | 0.24 | 0.28 |
| IMDNQ12 | 9.80 | 5.58 | 5.78 | 1.04 | 0.91 |
| IMDNQ13 | 5.37 | 9.95 | 3.31 | 0.33 | 0.62 |
| IMDNQ14 | 11.85 | 8.79 | 4.72 | 0.54 | 0.40 |
| IMDNQ15 | 0.000048 | 18.85 | 2.94 | 0.16 | 61250.00 |
| Am. B. | 5.26 | 22.26 | 1.18 | 0.05 | 0.22 |

[symbol]

indicates data missing or illegible when filed

### Cell Culture

Leishmania donovani isolated from human, Khartum, Sudan (ATCC#30030) were purchased from the American Type Culture Collection (ATCC). This clone was isolated in 1959. Balb/C 3T3 mouse fibroblasts used were the NIH 3T3 mouse embryonic fibroblast cells isolated from NIH Swiss mouse embryos and initiated in 1962 by G. Todaro and H. Green (Kuwahara et al., 1971; Todaro and Green, 1963).

Culturing Leishmania donovani Promastigotes

Approximately 5×10⁵ promastigotes/ml of L. donovani Khartum strain were inoculated into Yeager's Liver infusion tryptose (YLIT) medium or Brain Heart Infusion (BHI) broth (Difco), supplemented with glucose (2.5 mg/ml), 50 U/ml Pen-Strep and 0.005 mM Hemin at 25-26° C. and subcultured bi-weekly, Mikus et al., Parasitol Int., 48(3):265-9 (2000) (Mikus and Steverding, 2000).

Culturing Leishmania donovani Amastigotes

Amastigotes were grown in MAA/20 medium (Amastigote medium consisted of: M199 medium supplemented with 0.5% Tryptone broth, 0.01 mM bathocuproinedisulfonic acid, 0.108 mM L-Cysteine, 15 mM D-glucose, 0.685 mM L-glutamine, and 0.025 mM Hemin and 20% fetal calf serum (FCS) at a pH of 5.5). Amastigotes were maintained at 37° C. in the presence of 5% CO₂ and passaged bi-weekly (Mikus and Steverding, 2000).

### Culturing Fibroblasts

NIH 3T3 mouse fibroblasts were maintained in Roswell Park Memorial Institute (RPMI) medium supplemented with 10% Fetal calf serum, 50 units/ml penicillin, 50 units/ml streptomycin B in a humidified incubator at 37° C. in the presence of 5% CO₂ (Scala et al., 2010).

### Cell Harvesting

L. donovani

Parasites were collected by centrifugation for 10 minutes at 800×g, supernatant discarded, and parasites resuspended in 1× phosphate-buffered saline (PBS). The pelleted parasites were then resuspended in fresh medium and counted using a hemocytometer, Sen et al., Cell Death Diff., 11:934-936 (Sen et al., 2004a).

### NIH 3T3 Fibroblasts

Medium from the culturing T-75 flask was discarded, washed with 1×PBS 2× to remove medium completely. 3 ml of 1× Trypsin-EDTA was added to flask making sure that all the cells were covered with the Trypsin and the flask was incubated at 37° C. for ˜5 minutes. When more than 90% of the cells are detached, the trypsinization reaction was neutralized by the addition of 5 ml fresh culturing medium (Scala et al., 2010).

Antileishmanial Activity on L. donovani Promastigotes

The Leishmanicidal properties of fifteen naphthoquinone compounds (FIG. 1) on L. donovani promastigotes proliferation were assessed by the resazurin cell viability assay. Promastigotes from axenic culture were harvested during the exponential phase of growth (at least 5-day old culture), incubated at a concentration of 2×10⁵ cells/200 μl with and without dilutions of compounds in 96-well microtitre plates. Dilutions of compounds covering 0.03 μM to 44 μM were prepared. 2, 4, 6, 24 and 48 hrs after incubation at 26° C., plates were inspected under an inverted microscope to assure growth of the controls and sterile conditions. 20 μl Alamar Blue (12.5 mg resazurin dissolved in 100 ml distilled water (Sigma #R7017)) (Mikes and Steverding, 2000) were added to each well and plates were incubated for another 2 hrs. The plates were read with a microplate reader using a wavelength of 570 nm. Decrease in fluorescence (inhibition), were expressed as percentage of the fluorescence of control cultures and were plotted against the concentrations of compounds. The IC₅₀ values were calculated from the sigmoidal inhibition curves. Amphotericin B was used as a reference drug (Mikes and Steverding, 2000).

Antileishmanial Activity on L. donovani Amastigotes

Amastigotes were plated at a concentration of 2×10⁵ cells/2000 μl 24 hrs before treatment with various compounds. Amastigotes were treated with dilutions of compounds in 96-well microtitre plates. Dilutions of compounds ranging from 0.03 μM to 44 μM were prepared and added to amastigotes. Amastigotes were exposed to compounds for 48 hrs and were incubated at 37° C. During the period of exposure to compounds, plates were inspected under an inverted microscope to assure growth of the controls and sterile conditions. After the incubation period, 20 μl Alamar Blue (12.5 mg resazurin dissolved in 100 ml distilled water) (Mikes and Steverding, 2000) were added to each well and plates were incubated for another 2 hrs. The plates were read with a microplate reader using a wavelength of 570 nm. Decrease of fluorescence (inhibition) was expressed as percentage of the fluorescence of control cultures and was plotted against the concentrations of compounds. The IC₅₀ values were calculated from the sigmoidal inhibition curves. Amphotericin B was used as a reference drug.

### Cytotoxic Assessment of Compounds on NIH 3T3 BALB/c Mice Fibroblasts

Assays were performed to assess the cytotoxicity of these compounds in 96-well microtiter plates, with each well containing 100 μl of RPMI medium supplemented with 10% bovine calf scrum with 50 U/ml Pen-Strep. The cells were plated at a concentration of 4×10³ cells/well (4×10⁴ cells/ml). Fibroblast cells were then incubated with compounds for 48 hrs and inspected under an inverted microscope to assure growth of the controls and sterile conditions. 10 μl of resazurin solution (12.5 mg resazurin dissolved in 100 nil distilled water) were added to each well and the plates were incubated for another 2 hrs. The plates were read with a microplate reader at 570 nm. Experiments were done at least 3 times in triplicates (Scala et al., 2010).

### Animals

BALB/cAn NHsd (female, 10-11 weeks) mice were obtained from Charles Rivers Laboratories International Inc., Ballardvale, Wilmington, Mass., USA and weighed ˜20 g each at the time of infection. A standard mouse diet and regular clean tap water were supplied ad libitum. All animals were ‘specific pathogen free’. Experiments were conducted in accordance with Howard University's Institutional Animal Care and Use Committee (HU-IACUC) office approval.

Below are the compounds selected for use in this in vivo mice study.

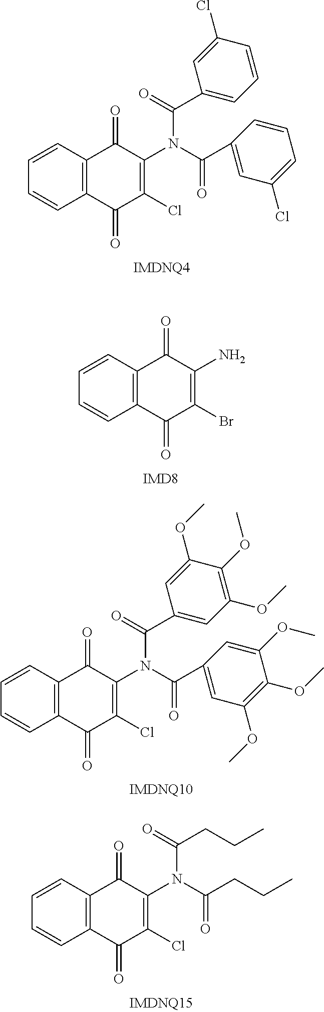

### In Vivo Visceral Leishmaniasis (VL) Model

Balb/c mice 10-11 weeks old were infected with L. donovani inoculated subcutaneously (SC) at the base of the tail (maximum 0.2 ml) with 1×10⁶ freshly harvested promastigotes. These represented a VL model which has been described previously (Yardley and Croft, 1999). After infection, mice were marked for individual identification and randomly allocated into groups of two. Seven days after infection, a mouse was sacrificed, and parasitic burden assessed based on microscopic enumeration of amastigotes (Leishman donovan bodies) against host cell nuclei on liver and spleen impression smears (Stauber et al., 1958). Blood was collected through cardiac puncture for use in all serum based assays.

Dosing began on the seventh day post-infection for 4 continuous days. As a positive control, one group did not receive any treatment. Dosing was administered subcutaneously at the base of the tail.

Table 2 shows four of the naphthoquinone compounds that were used in an in vivo study. It includes the different groups of mice and the concentrations of compounds used for the treatment of infections. A Representation of infections and treatments of BALB/c Mice is in Table 2. All mice were infected with 10⁶ log phase promastigotes in PBS. In each group with different concentrations, there were two mice (n=2). Four compounds (IMDNQ4, IMD8, IMDNQ10, and IMDNQ15) were used for the treatment of infected mice including the reference drug Amphotericin B. Two sets of controls were used. The positive controls that were infected but were not treated with any drug except PBS, and the negative controls that were not infected with the parasites, but injected with only PBS.

TABLE 2

| Groups and number | | Total |
|---|---|---|
| of mice | Infections | Treatments |
| 1. IDMDNQ4 | 1 × 10⁶ promastigotes/ | 5 mg/Kg |
| ♀ = 2/group | mouse | 20 mg/Kg |
| | | 50 mg/Kg |
| 2. IMD8 | 1 × 10⁶ promastigotes/ | 5 mg/Kg |
| ♀ = 2/group | mouse | 20 mg/Kg |
| | | 50 mg/Kg |
| 3. IMDNQ10 | 1 × 10⁶ promastigotes/ | 5 mg/Kg |
| ♀ = 2/group | mouse | 20 mg/Kg |
| | | 50 mg/Kg |
| 4. IMDNQ15 | 1 × 10⁶ promastigotes/ | 5 mg/Kg |
| ♀ = 2/group | mouse | 20 mg/Kg |
| | | 50 mg/Kg |
| 5. Am. B. | 1 × 10⁶ promastigotes/ | 20 mg/Kg |
| ♀ = 2 | mouse | |
| 6. Control 1 | 1 × 10⁶ promastigotes/ | PBS |
| ♀ = 2 | mouse | |
| 7. Control 2 | PBS | NONE |
| ♀ = 1 | | |

Mice were housed in cages kept in an animal room with controlled temperature and humidity. Mice were anesthetized each time doses were administered. Isofluoroethane was delivered in a precision vaporizer. Observation of mice for purposeful movements in cages after each dosing was done. Mice were euthanized during each blood and organ collection. Collected blood was spun after 1 hr. and the serum was stored in a −80° C. freezer for later testing.

Preparation of Antigens (L. donovani)

To make L. donovani crude antigens, L. donovani promastigotes and amastigotes were counted using a hemocytometer, spun at 800×g for 10 min, and washed three times with 1× phosphate buffered saline (PBS). After the final wash, parasites were resuspended in 1×PBS at a concentration of 1×10⁸ promastigotes (amastigotes)/ml and lysed by 5 freeze and thaw cycles or by sonication at maximum speed for 5 cycles of 10 seconds with cooling, and frozen at −80° C. until needed for assay (Burns et al., 1993; Zijlstra et al., 1998). Protein concentration was determined by the BIORAD assay.

### Determination of Humoral Response

5 μg/ml of antigen in 100 mM carbonate-bicarbonate buffer pH 7.4, were added to the wells of a 96 well polyvinyl plate (100 μl/well) and incubated at room temperature for 1 hr or overnight at 4° C. Wells were emptied, blocking buffer was added (200 μl/well), and wells were incubated at room temperature for 1 hr. Wells were emptied and 50 or 100 μl/well of diluted samples added (5× mice serum), and incubated for 1.5-2 hrs. Plates were washed 4× and an anti-mouse-IgG was diluted 1:500, and added (100 μl/well); these plates were incubated for 30 min in the dark and secondary antibody tagged to HRP was added at a dilution of 1:500. TMB-HRP developer was added 100 μl/well for 30 min in the dark and plates were read at 630 nm on a spectrophotometer (Zijlstra et al., 1998).

### Liver and Spleen Imprints for the Quantitation of Parasite Burden

The efficacy of compounds was assessed by microscopically determining the reduction in amastigotes burden Leishman donovan bodies (LDBs) within the liver and spleen. Impression smears were taken 14 days post infection (7 days after the start of treatment). Mice liver and spleen imprints were made by touching a freshly cut surface of the mice liver and spleen many times with a Poly-L-Lysine coated slide. The slides were air dried, fixed in “Diff Quick” fixative for 30 seconds, drained, stained with “Quick Diff” solution II for 30 seconds, drained, and then finally counterstaining with “Diff Quick” solution I for 30 seconds and then drained. Slides were rinsed in tap water to remove excess stain and rapidly dehydrated in absolute alcohol. Slides were examined by light microscopy, using ×1000 oil immersion. LDBs were determined by dividing the number of LDBs/number of cells nuclei x weight of organ in milligrams. This gave the number of “Leishman donovan units” (LDUs).

### In Vivo Cytotoxic Assessment of Compounds

Serum was collected from infected, treated and non-treated mice 14 days post infection and 7 days from the start of treatment for the determination of alanine amino transferase (ALT) and aspartate amino transferase (AST) activities. Assays were done in 96 well microtiter plates and samples and standards were run in duplicates or triplicates. For ALT, 2 μl of serum from infected treated and untreated samples were incubated with 10 μl of ALT substrate solution (1.78 g DL-alanine and 30 mg α-keto glutarate in 20 ml of phosphate buffer containing 1.25 ml of 0.4 M NaOH. Volume made up to 100 ml with buffer, pH 7.4. 1 ml of chloroform as preservative. Stable for 2 months at 2-8° C.), mixed and incubated at 37° C. for 30 minutes. 10 μl of dinitrophenolhydrazine (2,4 DNPH) was added. Blanks (ddH₂O) and negative (not infected) samples were then added. Samples were mixed and incubated at room temperature for 20 minutes. 100 μl of 0.4 M NaOH was added, mixed and incubated at room temperature for 5 min. Plates were read at 490 nm. For AST, 2 μl of test and positive (infected, treated and untreated) samples were incubated with 10 μl of AST substrate solution (2.66 g DL-aspartic acid and 30 mg α-keto glutarate in 20.5 ml of 1 M NaOH pH 7.4, volume made up to 100 ml with phosphate buffer. 1 ml of chloroform was added as preservative. This was stable for 2 months at 2-8° C.) and incubated at 37° C. for 1 hr. 10 μl of 2,4 DNPH added and mixed. 2 μl of negative controls (not infected) and blanks (ddH₂O) added and mixed and then incubated at room temperature for 20 min. 100 μl of 0.4 M NaOH was added, mixed and incubated at room temperature for 5 min and read at 490 nm. The enzyme activity was determined from the standard curve drawn using sodium pyruvate as standard solution (2 mM/ml pyruvate). Enzyme activities were expressed as units/ml. Reference ranges by this method are: AST; 0.096-3.8 U/ml and ALT; 0.112-3.0 U/ml. (Mallick et al., 2003; Reitman and Frankel, 1957).

### Statistical Analysis

All experiments were done in duplicate or triplicates and three different reproducible experiments were considered. The means and standard errors (S.E) were determined. Data were analyzed by one way ANOVA and t-test for multiple comparisons using Microsoft 2010 excel and GraphPad PRISM software version 5.0. P<0.05 was considered significant.

In Vitro Antileishmanial Activities of Naphthoquinone Compounds Against L. donovani Promastigotes and Amastigotes

The inhibitory concentrations (IC₅₀) of 15 naphthoquinone compounds (FIG. 1), which included imido-substituted 1,4-naphthoquinone compounds, were determined against L. donovani promastigotes and amastigotes with various concentrations ranging from 0.03 to 44 μM of tested compounds using Resazurin Assay.

FIG. 6 shows antileishmanial activities of these naphthoquinone compounds against promastigotes of L. donovani. The lower IC₅₀ value of tested naphthoquinone compounds compared to higher IC₅₀ value of Amphotericin B indicates that these compounds have more damaging effects on the growth of promastigotes of L. donovani in vitro. Results of analysis revealed that five of the naphthoquinone compounds: (1) IMDNQ1 with IC₅₀ value of 1.84±0.9 μM, (2) IMDNQ2 with IC₅₀ value of 2.27±1.4 μM, (3) IMD7 with IC₅₀ value of 2.00±0.3 μM, (4) IMD8 with IC₅₀ value of 4.4±0.4 μM, and (5) IMDNQ15 with IC₅₀ value of 4.8×10⁻⁵±0.004 μM compared to Amphotericin B with IC₅₀ value of 5.26±0.9 μM, were more potent growth inhibitors of promastigotes of L. donovani in vitro.

The level of antileishmanial activities (promastigotes killing or viabilities) may be indicated by variation in IC₅₀ values plotted in y-axis versus treatments (x-axis) with different concentrations of each tested compound ranging from 0.03 to 44 μM using Resazurin Assay. IC₅₀ values were calculated and expressed as the means±S.E (standard error) of three different experiments. The IC₅₀ value represents the concentration of tested naphthoquinone compound at which 50% promastigotes are killed after treatment.

In this test, the remaining 10 tested naphthoquinone compounds that showed lower anti-leishmanial activities against L. donovani promastigotes than Amphotericin B with IC₅₀ value of 5.26 μM are as follows: (1) IMDNQ3 with IC₅₀ value of 10.81±5.4 μM, (2) IMDNQ4 with IC₅₀ value of 6.82±2.3 μM, (3) IMDNQ5 with IC₅₀ value of 31.28±3.9 μM, (4) IMDNQ6 with IC₅₀ value of 9.66±30.0 μM, (5) IMDNQ9 with IC₅₀ value of 5.64±0.9 μM, (6) IMDNQ10 with IC₅₀ value of 7.0±1.0 μM, (7) IMDNQ11 with IC₅₀ value of 22.9±0.9 μM, (8) IMDNQ12 had an IC₅₀ value of 9.80±3.7 μM, (9) IMDNQ13 with IC₅₀ value of 5.37±0.001 μM, (10) IMDNQ11 with IC₅₀ value of 11.85±0.001 μM.

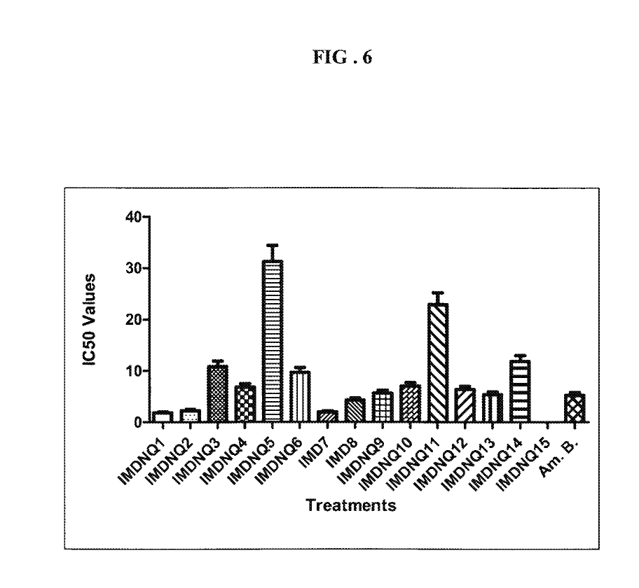

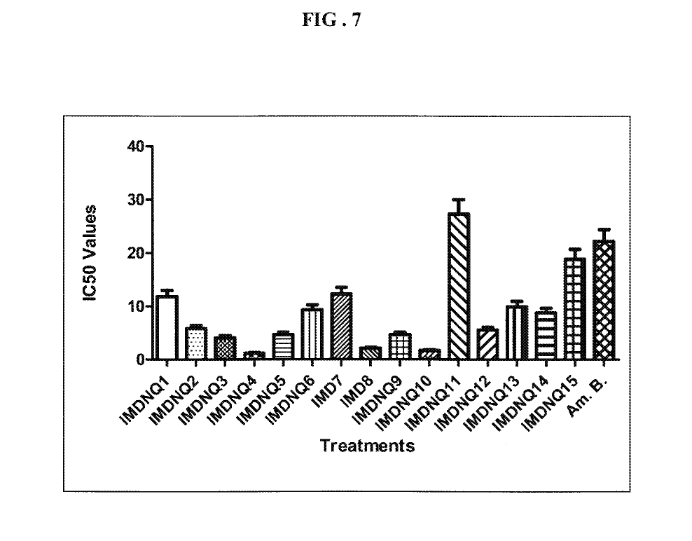

In vitro antileishmanial activities of the fifteen naphthoquinone compounds (FIG. 1), which include imido-substituted 1,4-naphthoquinone compounds, against L. donovani amastigotes are shown in FIG. 7. The lower IC₅₀ value of tested naphthoquinone compounds compared to higher IC₅₀ value of Amphotericin B indicates that these compounds have more damaging effects on the growth of amastigotes of L. donovani in vitro. In this test, Fourteen of the 15 tested naphthoquinone compounds have lower IC₅₀ values than Amphotericin B (22.26±0.001 μM), and are considered potent antileishmanial compounds against amastigotes of L. donovani in vitro. The compounds are: (1) IMDNQ1 with IC₅₀ value of 11.85±9.7 μM, (2) IMDNQ2 with IC₅₀ value of 5.83±6.2 μM, (3) IMDNQ3 with IC₅₀ value of 4.10±0.85 μM, (4) IMDNQ4 with IC₅₀ value of 1.19±1.11 μM, (5) IMDNQ5 with IC₅₀ value of 4.67±47.4 μM, (6) IMDNQ6 with IC₅₀ value of 9.32±6.1 μM, (7) IMD7 with IC₅₀ value of 12.28±17.3 μM, (8) IMD8 with IC₅₀ value of 2.07±1.0 μM, (9) IMDNQ9 with IC₅₀ value of 4.67±0.8 μM, (10) IMDNQ10 with IC₅₀ value of 1.69±0.02 μM, (11) IMDNQ12 with IC₅₀ value of 5.58±3.0 μM, (12) IMDNQ13 with value of 9.95±2.7 μM, (13) IMDNQ14 with IC₅₀ value of 8.79±39.2 μM, (14) IMDNQ15 with IC₅₀ value of 18.85±19.7 μM. The one compound that had a lower potency was IMDNQ11 with an IC₅₀ value of 27.31±26.3 μM compared to 22.26±0.001 μM for Amphotericin B.

The level of antileishmanial activities (amastigotes killing or viabilities) may be indicated by variation in IC₅₀ values plotted in y-axis versus treatments (x-axis) with different concentrations of each tested compound ranging from 0.03 to 44 μM using Resazurin Assay. IC₅₀ values were calculated and expressed as the means±S.E (standard error) of three different experiments. The IC₅₀ value represents the concentration of tested naphthoquinone compound at which 50% amastigotes are killed after treatment.

### Cytotoxic Effects of Naphthoquinone Compounds on Mouse Fibroblast Cells (NIH 3T3 BALB/c)

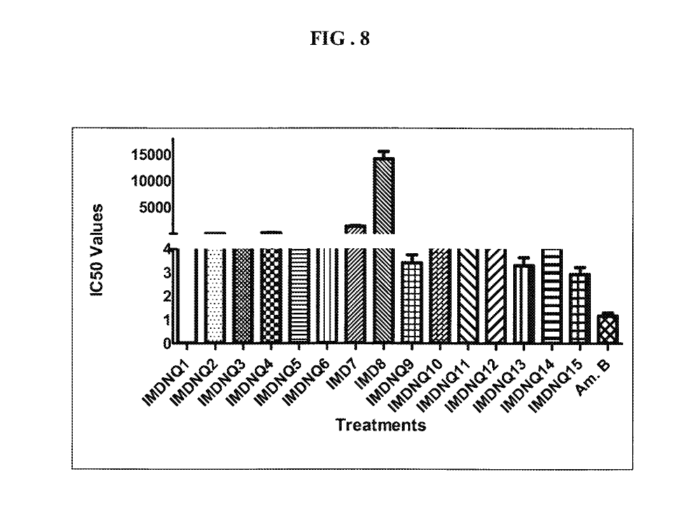

FIG. 8 shows the cytotoxic effects of the 15 naphthoquinone compounds (FIG. 1) on mouse fibroblasts. The higher the IC₅₀ value of each compound the less toxic is the compound. Data for Amphotericin B is included. The IC₅₀ values of all 15 naphthoquinone compounds are as follows: (1) IMDNQ1 with IC₅₀ value of 4.1±3.5 μM, (2) IMDNQ2 with IC₅₀ value of 78.75±10.92 μM, (3) IMDNQ3 with IC₅₀ value of 24.83±9.5 μM, (4) IMDNQ4 with value of 168.1±7.91 μM, (5) IMDNQ5 with IC₅₀ value of 4.7±0.41 μM, (6) IMDNQ6 with IC₅₀ value of 4.25±19.8 μM, (7) IMD7 with IC₅₀ value of 1448.56±0.84 μM, (8) IMD8 with IC₅₀ value of 14197.35±0.07 μM, (9) IMDNQ9 with IC₅₀ value of 3.41±1.1 μM, (10) IMDNQ10 with IC₅₀ value of 4.83±1.5 μM, (11) IMDNQ11 with IC₅₀ value of 6.41±2.9 μM, (12) IMDNQ12 with IC₅₀ value of 5.78±1.6 μM, (13) IMDNQ13 with IC₅₀ value of 3.31±0.001 μM, (14) IMDNQ14 with IC₅₀ value of 4.72±0.001 μM, and (15) IMDNQ15 with IC₅₀ value of 2.94±0.001 μM compared to 1.18±0.2 μM for Amphotericin B. All 15 naphthoquinone compounds showed higher IC₅₀ values against mouse fibroblasts compared to Amphotericin B (1.18±0.2 μM) indicating less toxicity of these compounds to mouse fibroblasts compared to Amphotericin B. Cells were plated in 96 well plates and treated with different concentrations of each compound ranging from 0.03 to 44 μM. Cell viability was determined using Resazurin Assay. The IC₅₀ values were calculated and expressed as means±S.E (standard error). Each bar represents an average of three experiments. Due to wide range of variations in IC₅₀ values, some histograms could not be plotted with S.E. The IC₅₀ value represents the concentration of tested naphthoquinone compound at which 50% mouse fibroblasts remained viable after treatment.

Selectivity Indices of Naphthoquinone Compounds Against L donovani Promastigotes and Amastigotes.

The fifteen naphthoquinone compounds, which include imido-substituted 1,4-naphthoquinone compounds as seen from FIG. 1, were evaluated for their cytotoxicities against promastigotes, amastigotes, and mouse fibroblasts. Selectivity indices (SIs) in promastigotes and amastigotes for each compound were calculated as the ratio between IC₅₀ values in fibroblast cells and IC₅₀ values in Leishmania promastigotes or amastigotes. The higher the selectivity index (SI) of the compound compared to that of Amphotericin B the more potent the compound.

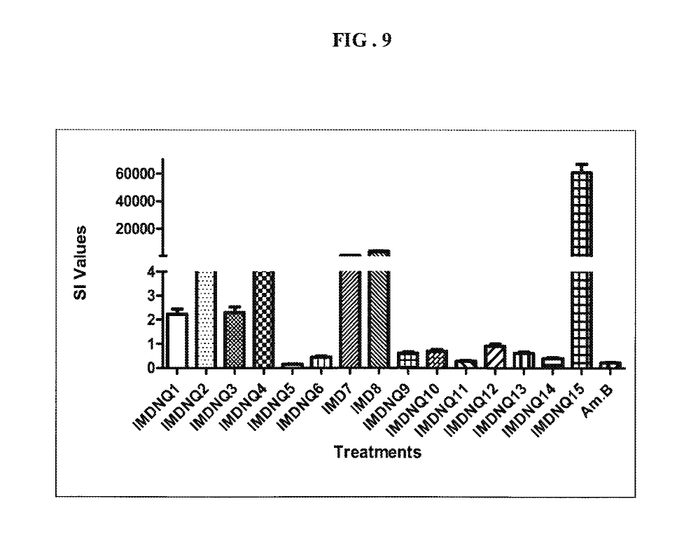

FIG. 9 shows SIs of the 15 naphthoquinone compounds against L. donovani promastigotes. Results of analysis showed that 14 of the 15 imido-substituted naphthoquinone compounds have higher SI values: (1) IMDNQ1 with an SI value of 2.23±1.09, (2) IMDNQ2 with an SI value of 34.69±26.4, (3) IMDNQ3 with an SI value of 2.30±1.05, (4) IMDNQ4 with an SI value of 24.65±12.51 (5) IMDNQ6 with an SI value of 0.44±0.47, (6) IMD7 with an SI value of 724.28±24.0, (7) IMD8 with an SI value of 3301.71±321.1, (8) IMDNQ9 with an SI value of 0.60±0.08, (9) IMDNQ10 with an SI value of 0.69±1.21, (10) IMDNQ11 with an SI value of 0.28±0.01, (11) IMDNQ12 with an SI value of 0.59±0.01, (12) IMDNQ13 with an SI value of 0.62±0.001, (13) IMDNQ14 with an SI value of 0.40±0.01, (14) IMDNQ15 with an SI value of 61250.00±438, compared to Amphotericin B with an SI value of 0.22±0.004. The remaining one compound, which showed a lower SI value than that of Amphotericin B was IMDNQ5 with an SI value of 0.15±0.3 compared to 0.22±0.004. NIH 3T3 Balb/c mice fibroblast cells were used to evaluate cytotoxicity of compounds in vitro. Selectivity index for each compound was calculated as a ratio between the IC₅₀ in fibroblast cells and the IC₅₀ in Leishmania promastigotes.

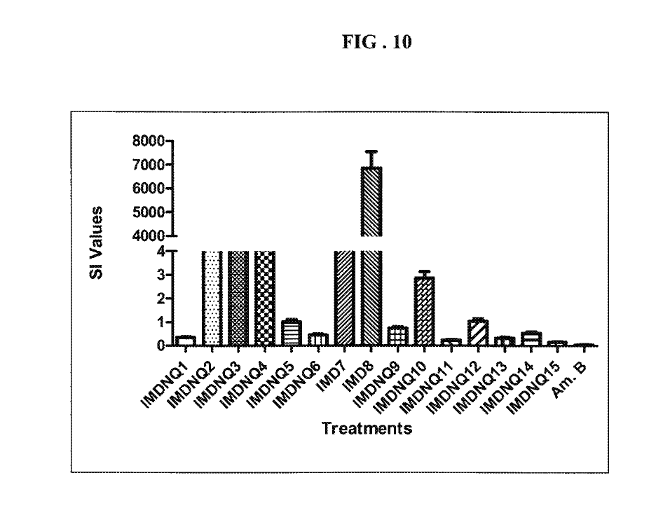

FIG. 10 shows SI values of the 15 naphthoquinone compounds (FIG. 1) against amastigotes of L. donovani. All 15 compounds have higher SI values and thus were more potent than Amphotericin B: (1) IMDNQ1 with SI value of 0.35±0.42, (2) IMDNQ2 with SI value of 13.51±33.49, (3) IMDNQ3 with SI value of 6.06±1.77, (4) IMDNQ4 with SI value of 141.26±56.8, (5) IMDNQ5 with SI value of 1.01±0.21, (6) IMDNQ6 with SI value of 0.46±0.38, (7) IMD7 with SI value of 117.96±2158, (8) IMD8 with SI value of 6858.62±3975.5, (9) IMDNQ9 with SI value of 0.73±0.13, (10) IMDNQ10 with SI value of 2.86±0.04, (11) IMDNQ11 with SI value of 0.23±0.42, (12) IMDNQ12 with SI value of 1.04±0.64, (13) IMDNQ13 with SI value of 0.33±0.09, (14) IMDNQ14 with SI value of 0.54±0.56, (15) IMDNQ15 with SI value of 0.16±0.09, compared to Amphotericin B with SI value of 0.05±0.001. NIH 3T3 Balb/c mice fibroblast cells were used to evaluate cytotoxicity of compounds in vitro, which allowed for the determination of in vitro selectivity indices. The selectivity indices in amastigotes for each compound was calculated as a ratio between cytotoxicity IC₅₀ in fibroblast cells and antileishmanial activities against amastigotes. Some histograms could not be plotted with S.E. due to wide range of variations in SI values.

Data in Table 3 were used to generate FIGS. 6, 7, and 8; the Table shows IC₅₀ values for the 15 naphthoquinone compounds in promastigotes, amastigotes, and mouse fibroblasts. The lower the IC₅₀ value for each compound against promastigotes and amastigotes, the more potent is the compound. An IC₅₀ value of Amphotericin B is included.

TABLE 3
In Vitro Cytotoxic Activities of the tested Naphthoquinone
Compounds and Amphotericin B against L. donovani Promastigotes,
Amastigotes, and mouse fibroblasts NIH 3T3.

| Tested | IC₅₀ (μM) | | |
|---|---|---|---|
| Compounds | Promastigotes | Amastigotes | Fibroblasts |
| IMDNQ1 | 1.84 ± 0.9 | 11.85 ± 9.7 | 4.1 ± 3.5 |
| IMDNQ2 | 2.27 ± 1.4 | 5.83 ± 6.2 | 78.75 ± 10.92 |
| IMDNQ3 | 10.81 ± 5.4 | 4.10 ± 0.85 | 24.83 ± 9.5 |
| IMDNQ4 | 6.82 ± 2.3 | 1.19 ± 1.11 | 168.1 ± 7.91 |
| IMDNQ5 | 31.28 ± 3.9 | 4.67 ± 47.4 | 4.7 ± 0.41 |
| IMDNQ6 | 9.66 ± 30.0 | 9.32 ± 6.1 | 4.25 ± 19.8 |
| IMD7 | 2.00 ± 0.3 | 12.28 ± 17.3 | 1448.56 ± 0.84 |
| IMD8 | 4.3 ± 0.4 | 2.07 ± 1.0 | 14197.35 ± 0.07 |
| IMDNQ9 | 5.64 ± 0.8 | 4.67 ± 0.8 | 3.41 ± 1.1 |
| IMDNQ10 | 7.0 ± 1.0 | 1.69 ± 0.02 | 4.83 ± 1.5 |
| IMDNQ11 | 22.9 ± 0.8 | 27.31 ± 26.3 | 6.41 ± 2.9 |
| IMDNQ12 | 9.80 ± 3.7 | 5.58 ± 3.0 | 5.78 ± 1.6 |
| IMDNQ13 | 5.37 ± 0.001 | 9.95 ± 2.7 | 3.31 ± 0.001 |
| IMDNQ14 | 11.85 ± 0.001 | 8.79 ± 39.2 | 4.72 ± 0.001 |
| IMDNQ15 | 4.8e−5 ± 0.004 | 18.85 ± 19.7 | 2.94 ± 0.001 |
| Am. B. | 5.26 ± 0.9 | 22.26 ± 0.001 | 1.18 ± 0.2 |

Data from Table 4 were used to generate FIGS. 9 and 10. The higher the SI value of the compound, the more selective is the naphthoquinone compound tested. Data for Amphotericin B is included.

TABLE 4
In Vitro Selectivity Indices of 15 Imido-substituted
Naphthoquinone Compounds and Amphotericin B against
L. donovani Promastigotes and Amastigotes.

| | |
|---|---|
| Tested | SI Values |

| Compounds | Amastigotes | Promastigotes |
|---|---|---|
| IMDNQ1 | 0.35 ± 0.42 | 2.23 ± 1.09 |
| IMDNQ2 | 13.51 ± 33.49 | 34.69 ± 26.4 |
| IMDNQ3 | 6.06 ± 1.77 | 2.30 ± 1.05 |
| IMDNQ4 | 141.26 ± 56.8 | 24.65 ± 12.51 |
| IMDNQ5 | 1.01 ± 0.21 | 0.15 ± 0.3 |
| IMDNQ6 | 0.46 ± 0.38 | 0.44 ± 0.47 |
| IMD7 | 117.96 ± 2158 | 724.28 ± 24.0 |
| IMD8 | 6858.62 ± 3975.5 | 3301.71 ± 321.1 |
| IMDNQ9 | 0.73 ± 0.13 | 0.60 ± 0.08 |
| IMDNQ10 | 2.86 ± 0.04 | 0.69 ± 1.21 |
| IMDNQ11 | 0.23 ± 0.42 | 0.28 ± 0.01 |
| IMDNQ12 | 1.04 ± 0.64 | 0.59 ± 0.01 |
| IMDNQ13 | 0.33 ± 0.09 | 0.62 ± 0.001 |
| IMDNQ14 | 0.54 ± 0.56 | 0.40 ± 0.01 |
| IMDNQ15 | 0.16 ± 0.09 | 61250.00 ± 438 |
| Am. B | 0.05 ± 0.001 | 0.22 0.004 |

*SI (promastigotes) = IC₅₀ (mouse fibroblast) ÷ IC₅₀ (promastigotes)
*SI (amastigotes) = IC₅₀ (mouse fibroblast) ÷ IC₅₀ (amastigotes)

Dose and Time-Dependent Inhibitory Effect of Naphthoquinone Compounds on L. donovani Promastigotes.

In order to determine the effect of the 15 naphthoquinone compounds (FIG. 1) on the membrane polysaccharides present only in promastigotes, a dose and time-dependent inhibition was carried out at different durations of observation—2 hours, 4 hours, 6 hours, and 24 hours. For each duration of observation the lower the IC₅₀ value of the compound compared to that of Amphotericin B, the more potent the compound.

Results of the 2-hour duration of observation are shown in FIG. 11. Fourteen of the 15 compounds were less potent than Amphotericin B: (1) IMDNQ1 with IC₅₀ value of 5.53±0.3 μM, (2) IMDNQ2 with IC₅₀ value of 5.04±1.3 μM, (3) IMDNQ3 with IC₅₀ value of 10.96±12.9 μM (4) IMDNQ4 with IC₅₀ value of 3.52±3.6 μM, (5) IMDNQ5 with IC₅₀ value of 19.02±1.5 μM, (6) IMDNQ6 with IC₅₀ value of 10.39±1.3 μM, (7) IMD7 with IC₅₀ value of 7.18×10⁵±24.2 μM, (8) IMDNQ9 with IC₅₀ value of 77.34±118.8 μM, (9) IMDNQ10 with IC₅₀ value of 12.82±4.3 μM (10) IMDNQ11 with IC₅₀ value of 198.10±16.8 μM, (11) IMDNQ12 with IC₅₀ value of 41.10±11.9 μM, (12) IMDNQ13 with IC₅₀ value of 35.80±0.01 μM, (13) IMDNQ14 with IC₅₀ value of 5046.30±0.01 μM, (14) IMDNQ15 with IC₅₀ value of 8.60±0.01 μM compared to Amphotericin B with IC₅₀ value of 3.09±2.0 μM. The remaining one compound that showed a higher potency was IMD8 with IC₅₀ of 0.021±0.001 μM compared to Amphotericin B with IC₅₀ of 3.09±2.0 μM. A time and dose-dependent inhibitory effect of all 15 imido-substituted naphthoquinone compounds was assessed on their activity on promastigotes of L. donovani. Promastigotes were plated in 96-well plates and incubated in different concentrations of each compound (0.03 to 44 μM), incubated for two hours and viabilities assayed by the resazurin assay. A two hour exposure was assessed by calculation of IC₅₀ values of each compound. Values are means±S.E. (Standard Error). Due to wide range of variations in IC₅₀ values, some histograms could not be plotted with S.E.

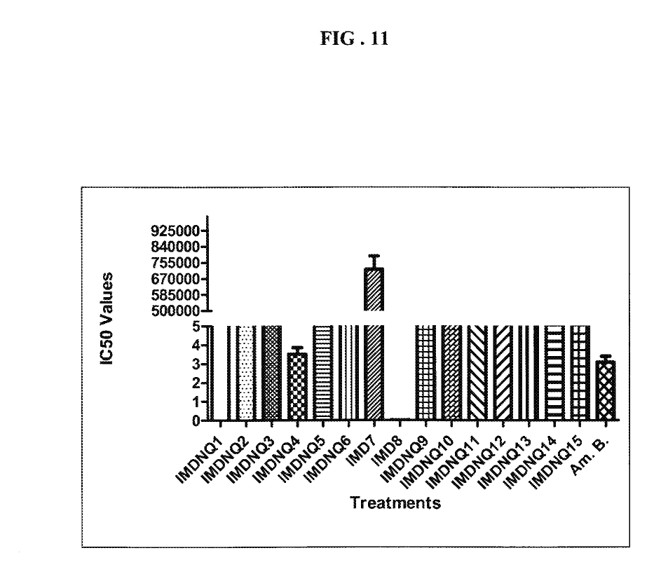

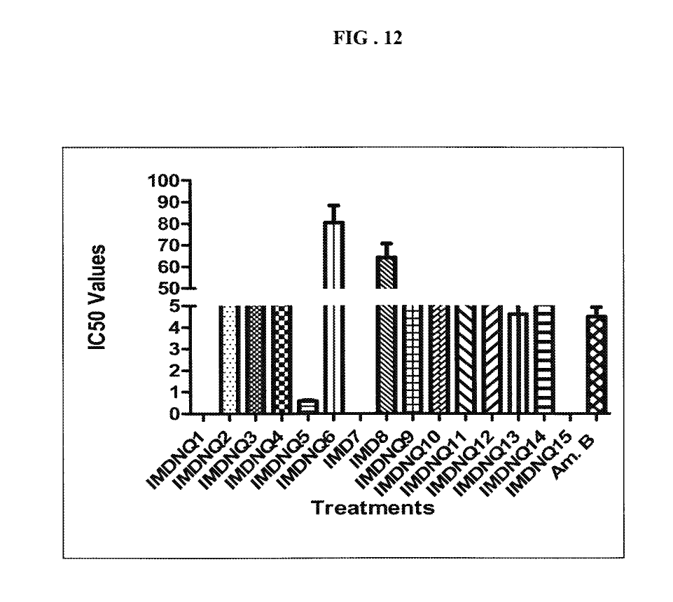

Results of the 4-hour duration of observation are shown in FIG. 12. Twelve of the 15 compounds were less potent than Amphotericin B: (1) IMDNQ1 with IC₅₀ of 1101.70±50.3 μM, (2) IMDNQ2 with IC₅₀ of 14.62±7.0 μM, (3) IMDNQ3 with IC₅₀ of 41.35±0.21 μM, (4) IMDNQ4 with IC₅₀ of 21.49±9.6 μM, (5) IMDNQ6 with IC₅₀ of 80.32±0.76 μM, (6) IMD8 with IC₅₀ of 64.17±11.8 μM, (7) IMDNQ9 with IC₅₀ of 23.32±9.2 μM, (8) IMDNQ10 with IC₅₀ of 11.30±0.6 μM, (9) IMDNQ11 with IC₅₀ of 22.70±14.2 μM, (10) IMDNQ12 with IC₅₀ of 13.87±6.4 μM, (11) IMDNQ13 with IC₅₀ of 4.62±0.01 μM, (12) IMDNQ14 with IC₅₀ of 9.07±1.9 μM compared to Amphotericin B with IC₅₀ of 4.49±0.3 μM. The three compounds that showed higher activity than Amphotericin B are: (1) IMDNQ5 with IC₅₀ of 0.57±4.1 μM, (2) IMD7 with IC₅₀ of 5.9×10⁻²⁴±3.4 μM, (3) IMDNQ15 with IC₅₀ of 8.95×10⁻¹¹±1.3×10⁻¹⁰ μM compared to Amphotericin B with IC₅₀ of 4.49±0.3 μM (P=4.3×10⁻²²). A time and dose-dependent inhibitory effect of all 15 imido-substituted naphthoquinone compounds was assessed on their activity on promastigotes of L. donovani. Promastigotes were plated in 96-well plates and incubated in different concentrations of each compound (0.03 to 44 μM), incubated for four hours and cell viabilities assayed by the resazurin assay. A four hour exposure was assessed by calculation of IC₅₀ values of each compound. Values are means±S.E. (Standard Error).

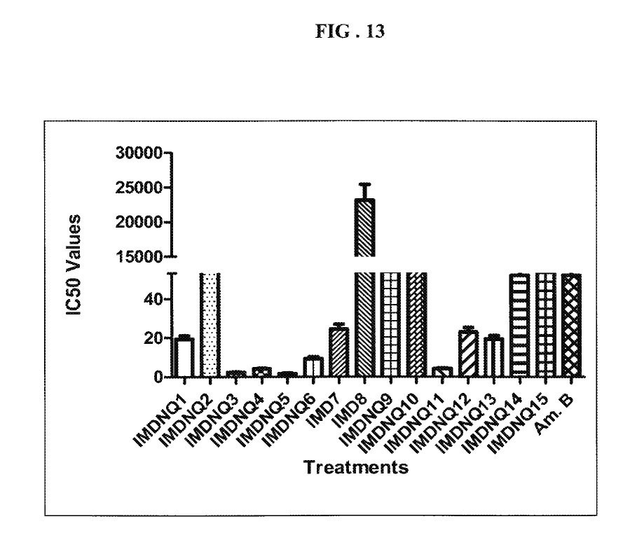

Results of the 6-hour duration of observation are shown in FIG. 13. Five of the 15 compounds were less potent than Amphotericin B: (1) IMDNQ2 with IC₅₀ of 116.35±3.2 μM, (2) IMD8 with IC₅₀ of 2.31×10⁴±25.9 μM, (3) IMDNQ9 with IC₅₀ of 123.38±35.1 μM, (4) IMDNQ10 with IC₅₀ of 242.61±24.5 μM, (5) IMDNQ15 with an IC₅₀ value of 116.35±0.07 μM compared to IC₅₀ of 52.02±12.2 μM for Amphotericin B. IMDNQ14 was just as potent as Amphotericin B; It had IC₅₀ of 52.02±0.01 μM compared to IC₅₀ of 52.02±12.2 μM for Amphotericin B. The remaining nine compounds were less potent than Amphotericin B. They are: (1) IMDNQ1 with IC₅₀ of 19.21±18.4 μM, (2) IMDNQ3 with IC₅₀ of 2.32±1.9 μM, (3) IMDNQ4 with IC₅₀ of 4.11±6.3 μM, (4) IMDNQ5 with IC₅₀ value of 1.66±0.5 μM, (5) IMDNQ6 with IC₅₀ of 9.25±0.8 μM, (6) IMD7 with IC₅₀ of 24.44±6.9 μM, (7) IMDNQ11 with IC₅₀ of 4.06±1.4 μM, (8) IMDNQ12 with IC₅₀ of 22.94±12.3 μM, (9) IMDNQ13 with IC₅₀ of 19.21±0.01 μM, compared to IC₅₀ of 52.02±12.2 μM for Amphotericin B. A time and dose-dependent inhibitory effect of all 15 imido-substituted naphthoquinone compounds was assessed on their activity on promastigotes of L. donovani. Promastigotes were plated in 96-well plates and incubated in different concentrations of each compound (0.03 to 44 μM), incubated for six hours and cell viabilities assayed by the resazurin assay. A six hour exposure was assessed by calculation of IC₅₀ values of each compound. Values are means±S.E. (Standard error). Some histograms could not be plotted with S.E due to wide range of variations in IC₅₀ values.

Results of the 24-hour duration of observation are shown in FIG. 14. Twelve of the 15 compounds were less potent than Amphotericin B: (1) IMDNQ1 with IC₅₀ of 5.28±1.8 μM, (2) IMDNQ2 with IC₅₀ of 8.22±3.9 μM, (3) IMDNQ5 with IC₅₀ of 5.85±2.2 μM, (4) IMDNQ6 with IC₅₀ of 7.27±2.7 μM, (5) IMD7 with IC₅₀ of 7.93±0.7 μM, (6) IMD8 with IC₅₀ of 12.03±0.8 μM, (7) IMDNQ9 with IC₅₀ of 7.23±0.7 μM, (8) IMDNQ10 with IC₅₀ of 9.80±0.1 μM, (9) IMDNQ11 with IC₅₀ of 8.82±2.0 μM, (10) IMDNQ12 with IC₅₀ of 5.41±0.3 μM, (11) IMDNQ13 with IC₅₀ of 10.05±0.01 μM, (12) IMDNQ14 with IC₅₀ of 7.19±0.1 μM, compared to Amphotericin B with IC₅₀ of 4.10±0.4 μM. The three compounds that were more potent than Amphotericin B: (1) IMDNQ3 with IC₅₀ of 3.23±5.3 μM, (2) IMDNQ4 with IC₅₀ of 3.96±1.3 μM, (3) IMDNQ15 with IC₅₀ of 3.96±0.0 μM compared to Amphotericin B with IC₅₀ of 4.10±0.4 μM (P=2.6×10⁻⁴⁷). A time and dose-dependent inhibitory effect of all 15 imido-substituted naphthoquinone compounds was assessed on their activity on promastigotes of L. donovani. Promastigotes were plated in 96-well plates and incubated in different concentrations of each compound (0.03 to 44 μM), incubated for twenty four hours and cell viabilities assayed by the resazurin assay. A twenty four hour exposure was assessed by calculation of IC₅₀ values of each compound. Values are means±S.E. (Standard Error).

The data in Table 5 summarize the observations shown in FIGS. 11, 12 13 and 14. The IC₅₀ values are for the 15 compounds at 2-hour, 4-hour, 6-hour, and 24-hour duration of observation.

TABLE 5
In Vitro Effects of 15 naphthoquinone compounds on the membrane polysaccharides of
promastigotes of L. donovani. A dose-dependent activity of compounds and amphotericin
B against L. donovani Promastigotes at 2, 4, 6, and 24 hours of exposure.

| Tested | Antileishmanial Activity IC₅₀ (μM) | | | |
|---|---|---|---|---|
| Compounds | 2 hours | 4 hours | 6 hours | 24 hours |
| IMDNQ1 | 5.53 ± 0.3 | 1101.70 ± 50.3 | 19.21 ± 18.4 | 5.28 ± 1.8 |
| IMDNQ2 | 5.04 ± 1.3 | 14.62 ± 7.0 | 116.35 ± 3.2 | 8.22 ± 3.9 |
| IMDNQ3 | 10.96 ± 12.9 | 41.35 ± 0.21 | 2.32 ± 1.9 | 3.23 ± 5.3 |
| IMDNQ4 | 3.52 ± 3.6 | 21.49 ± 9.6 | 4.11 ± 6.3 | 3.96 ± 1.3 |
| IMDNQ5 | 19.02 ± 1.5 | 0.57 ± 4.1 | 1.66 ± 0.5 | 5.85 ± 2.2 |
| IMDNQ6 | 10.39 ± 1.3 | 80.32 ± 0.76 | 9.25 ± 0.8 | 7.27 ± 2.7 |
| IMD7 | 7.18 × 10⁵ ± 24.2 | 5.9 × 10⁻²⁴ ± 3.4 | 24.44 ± 6.9 | 7.93 ± 0.7 |
| IMD8 | 0.021 ± 0.001 | 64.17 ± 11.8 | 2.31 × 10⁴ ± 25.9 | 12.03 ± 0.8 |
| IMDNQ9 | 77.34 ± 19.8 | 23.32 ± 9.2 | 123.38 ± 35.1 | 7.23 ± 0.7 |
| IMDNQ10 | 12.82 ± 4.3 | 11.30 ± 0.6 | 242.61 ± 24.5 | 9.80 ± 0.1 |
| IMDNQ11 | 198.10 ± 16.8 | 22.70 ± 14.2 | 4.06 ± 1.4 | 8.82 ± 2.0 |
| IMDNQ12 | 41.10 ± 11.9 | 13.87 ± 6.4 | 22.94 ± 12.3 | 5.41 ± 0.3 |
| IMDNQ13 | 35.80 ± 0.01 | 4.62 ± 0.01 | 19.21 ± 0.01 | 10.05 ± 0.01 |
| IMDNQ14 | 5046.30 ± 0.01 | 9.07 ± 1.9 | 52.02 ± 0.01 | 7.19 ± 0.1 |
| IMDNQ15 | 8.60 ± 0.01 | 8.95 × 10⁻¹¹ ± 1.3 × 10⁻¹⁰ | 116.35 ± 0.07 | 3.96 ± 0.0 |
| Am. B. | 3.09 ± 2.0 | 4.49 ± 0.3 | 52.02 ± 12.2 | 4.10 ± 0.4 |

### Visceral Leishmaniasis BALB/c Mouse Model

The purpose of the test was to show the presence of amastigotes in the liver and spleen of infected BALB/c mice. BALB/c mice models of visceral leishmaniasis were generated by infecting mice with 10⁶ log phase promastigotes in 200 μl 1×PBS. Infectivity was confirmed by making liver and spleen imprints of infected mice seven days post infection before the start of treatment. Slides were stained using the “Diff Quick” stain. Positive infectivity was confirmed by the presence of amastigotes in stained slides compared to that of the uninfected slides.

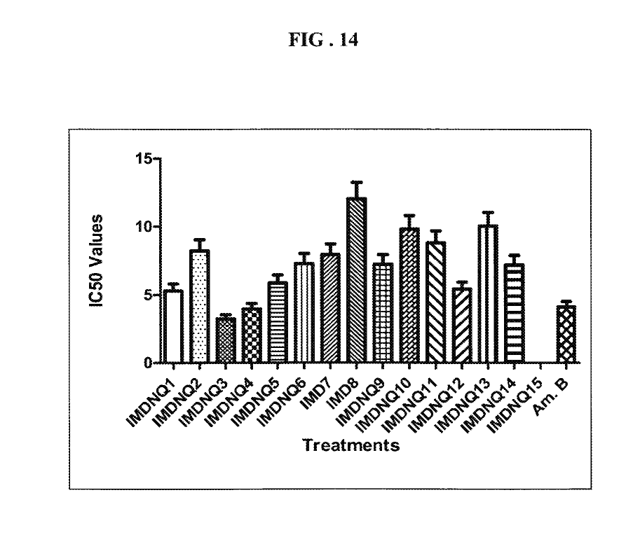

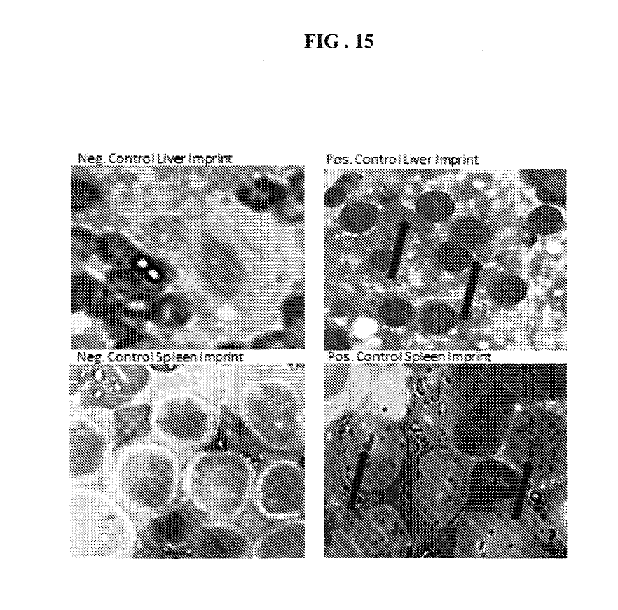

FIG. 15 shows the presence of amastigotes in the liver (pos. Control Liver Imprint—tip of arrows) after seven days post infection compared with that of the uninfected liver imprints (Neg. Control Liver Imprints). The lower panel shows the presence of amastigotes in spleen imprints (pos. Control Spleen Imprints—tip of arrows) seven days post infection compared with that of uninfected negative control (Neg. Control Spleen Imprint). Confirmation of the generation of a VL model was confirmed after injecting Balb/c mice with 1×10⁶ promastigotes in 200 μl of PBS at the base of their tail. Seven days post infection, a mouse was sacrificed and infectivity was confirmed by detection of Leishman Donovan units in liver and spleen, and stained with “Diff Quick” stain.

### Seroprevalence of IgG in BALB/c Mice

The purpose of the test was to confirm positive infectivity through levels of serum IgG in infected BALB/c mice. A high serum IgG level is predictive of an infection with visceral leishmaniasis. The results are expressed as percent increase or percent decrease in IgG levels. Four compounds with three different concentrations each were used for the tests. Amphotericin B had only one concentration.

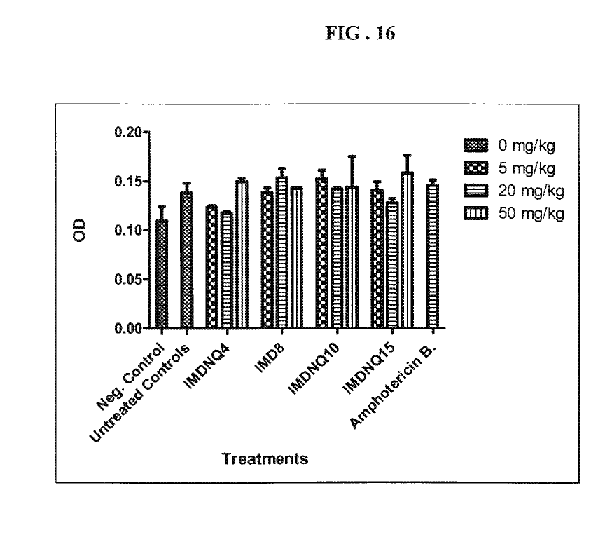

FIG. 16 shows the levels of serum IgG in uninfected, infected untreated and infected but treated BALB/c mice. Serum levels of IgG in infected but untreated mice increased by 20.36% compared to that of uninfected mice. For IMDNQ4: 5 mg/kg had 11.34% increase in IgG level; 20 mg/kg had 6.81% increase in IgG level; 50 mg/kg had 26.76% increase in IgG level. For IMD8: 5 mg/kg had 20.94% increase in IgG level; 20 mg/kg had 28.66% increase in IgG; 50 mg/kg had 23.16% increase in IgG level compared to that of untreated controls. For IMDNQ10: 5 mg/kg had 27.96% increase, 20 mg/kg had 21.05% increase, 50 mg/kg had 21.79% increase in IgG level compared to that of untreated controls. For IMDNQ15: 5 mg/kg had 21.79% increase, 20 mg/kg had 14.12% increase in IgG level, 50 mg/kg had 30.70% increase in IgG level compared to that of untreated controls. For Amphotericin B: 20 mg/kg had 24.74% increase in IgG level compared to that of untreated controls. Specific-parasite IgG antibodies produced by BALB/c mice inoculated with 10⁶ infective concentrations of Leishmania donovani. Mice (n=2 per group) were infected subcutaneously at the base of their tail with log phase promastigotes. The negative control group received only PBS and did not present any infection. Serum from mice was obtained fourteen days post-infection and tested individually by ELISA to determine the specific levels of L. donovani IgG. Bars indicate the mean±S.E. (Standard Error) from two mice.

Evaluation of Splenic and Liver Parasite Burden after Treatment with Selected Compounds

The purpose of the test was to evaluate splenic and liver parasite burden after treatment with selected compounds. This was done by making imprints and calculating the number of Leishman Donovan Units (LDUs) in cells. LDUs are calculated by counting the number of parasites per cell nuclei (number of amastigotes in cells÷number of cells) multiplied by the weight of the organ in milligrams. The lower the LDU value compared to that of untreated controls, the more effective the compound is at that concentration for the treatment of VL. Effectiveness of compounds in the treatment of VL is expressed as LDU numbers and as percent increases or percent decrease in LDUs. Percent increase in LDU means that the number of parasites increased compared to the untreated controls and percent decrease means that the number of parasites decreased compared to the untreated controls.

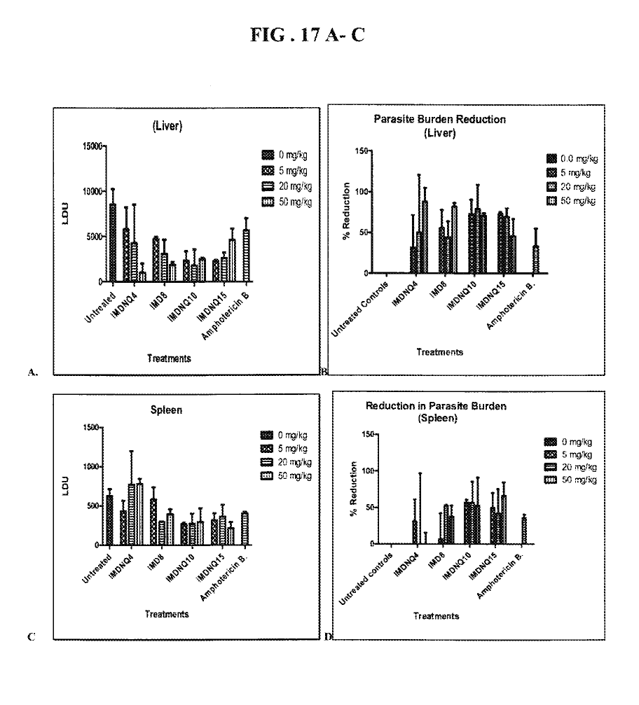

FIG. 17s A and B show suppressions in the liver parasite burden. All four compounds were effective in the treatment of VL. For IMDNQ4: 5 mg/kg had LDU value of 5823.8±3378, 31.79% reduction in parasite burden; 20 mg/kg had LDU value of 4266.0±6033, a 50.04% reduction in parasite burden; 50 mg/kg had LDU value of 1003.8±1420, an 88.24% reduction in parasite burden. For IMD8: 5 mg/kg had LDU value of 4752.5±293, a 56.66% reduction in parasite burden; 20 mg/kg had LDU value of 3099.9±2197, a 44.34% reduction in parasite burden; 50 mg/kg had LDU value of 1869.0±420 an 81.89% reduction in parasite burden. For IMDNQ 10: 5 mg/kg had LDU value of 2313.0±1521 a 72.91% reduction in parasite burden; 20 mg/kg had LDU value of 1794.0±2537, a 78.99% reduction in parasite burden; 50 mg/kg had an LDU value of 2478.0±229, a 70.98% reduction in parasite burden. For IMDNQ15: 5 mg/kg had LDU value of 2267.5±191, a 73.44% reduction in parasite burden; 20 mg/kg had LDU value of 2598.5±89, a 69.57% reduction in parasite burden; 50 mg/kg had LDU value of 5681.0±1785, a 45.92% reduction in parasite burden. Amphotericin B had LDU value of 5823.8 compared to 8538.5±2423 for the untreated control, a 33.47% reduction in parasite burden.

FIGS. 17 C and D show the suppressions in the liver parasite burden. In the spleen, three of the four compounds (IMDNQ4, IMD8, and IMDNQ 10) showed lower LDU values at each of these concentrations compared to that of untreated controls. For IMDNQ4: 5 mg/kg had LDU value of 433.3±188, a 30.79% reduction in parasite burden; 20 mg/kg had LDU value of 770.0±608, an 18.7% increase in parasite burden; 50 mg/kg had LDU value of 780.3±96, a 19.8% increase in parasite burden. For IMD8 5 mg/kg had LDU value of 585.3±222, a 6.51% reduction in parasite burden; 20 mg/kg had LDU value of 297.8±9.5, a 52.44% reduction in parasite burden; 50 mg/kg had an LDU value of 391.5±96, an 81.89% reduction in parasite burden. For IMDNQ10: 5 mg/kg had LDU value of 269.5±25, a 72.91% reduction in parasite burden; 20 mg/kg had LDU value of 273.0±180, a 78.99% reduction in parasite burden; 50 mg/kg had LDU value of 297.0±242, a 70.98% reduction in parasite burden; For IMDNQ15: 5 mg/kg had LDU value of 315.0±129, a 73.44% reduction in parasite burden; 20 mg/kg had LDU value of 363.3±210, a 67.57% reduction in parasite burden; 50 mg/kg had LDU value of 213.0±115, a 45.92% reduction in parasite burden. Amphotericin B 20 mg/kg had LDU value of 402.0±30, a 33.0% reduction in parasite burden. L. donovani-infected BALB/c mice received 10⁶ parasites and were treated seven days post infection for four days with respective compounds including Amphotericin B. Mice were sacrificed fourteen days after infection. Hepatic (A), and splenic (C) parasite burden were quantified as LDUs. Data represent means S.E. (Standard Error) for two animals per group. Data were tested by ANOVA. Differences between means were assessed for statistical significance by T-test at P=0.05 (n=2 per group).

Table 6 is a summary of FIGS. 16 and 17. The effectiveness of the naphthoquinone compounds in the treatment of VL is shown in the form of LDUs as an expression of the reduction in parasite burden after treatment. Results are also shown as changes in the percentage of LDU compared to that of untreated controls and Amphotericin B.

TABLE 6
Suppression of VL in Balb/c mice treated with Four Selected Compounds.
As compared with baseline values, suppressions were defined as a decrease
in Leishman Donovan Units (LDUs) of >0% values and above indicate
reductions in the parasite burden compared to untreated controls.

| | Treatments | | | Parasite | Parasite |
|---|---|---|---|---|---|
| | Total dose | LDUs | LDUs | Suppression | Suppression |
| Compounds | (mg/kg) | (Liver) | (Spleen) | (%)--Liver | (%)--Spleen |
| Untreated | None | 8538.5 ± 2423 | 626.0 ± 122 | 0 | 0 |
| control | | | | | |
| IMDNQ4 | 5 | 5823.8 ± 3378 | 433.3 ± 188 | 31.79 | 30.79 |
| | 20 | 4266.0 ± 6033 | 770.0 ± 608 | 50.04 | 0 |
| | 50 | 1003.8 ± 1420 | 780.3 ± 96 | 88.24 | 0 |
| IMD8 | 5 | 4752.5 ± 293 | 585.3 ± 222 | 55.66 | 6.51 |
| | 20 | 3099.9 ± 2197 | 297.8 ± 9.5 | 44.34 | 52.44 |
| | 50 | 1869.0 ± 420 | 391.5 ± 96 | 81.89 | 37.46 |
| IMDNQ10 | 5 | 2313.0 ± 1521 | 269.5 ± 25 | 72.91 | 56.95 |
| | 20 | 1794.0 ± 2537 | 273.0 ± 180 | 78.99 | 56.39 |
| | 50 | 2478.0 ± 229 | 297.0 ± 242 | 70.98 | 52.56 |
| IMDNQ15 | 5 | 2267.5 ± 191 | 315.0 ± 129 | 73.44 | 49.68 |
| | 20 | 2598.5 ± 889 | 363.3 ± 210 | 69.57 | 41.97 |
| | 50 | 4618.0 ± 1785 | 213.0 ± 115 | 45.92 | 65.97 |
| Am. B. | 20 | 5681.0 ± 1868 | 402.3 ± 30 | 33.47 | 35.74 |

n = 2 for each group.

### Toxicological Effects of Naphthoquinone Derivatives in BALB/c Mice Model

In order to assess in vivo cytotoxicity of four naphthoquinone compounds (IMDNQ4, IMD8, IMDNQ 10, and IMDNQ 15), serum aspartate aminotransferase (AST), and alanine aminotransferase (ALT) levels were analyzed and compared to those in untreated controls. The assay range for AST is 0.096-3.8 U/ml (mean assay range is 1.95 U/ml) and for ALT assay, 0.112-3.0 U/ml (mean assay range is 1.56 U/ml). The results are expressed as percent increase or decrease in AST or ALT levels.

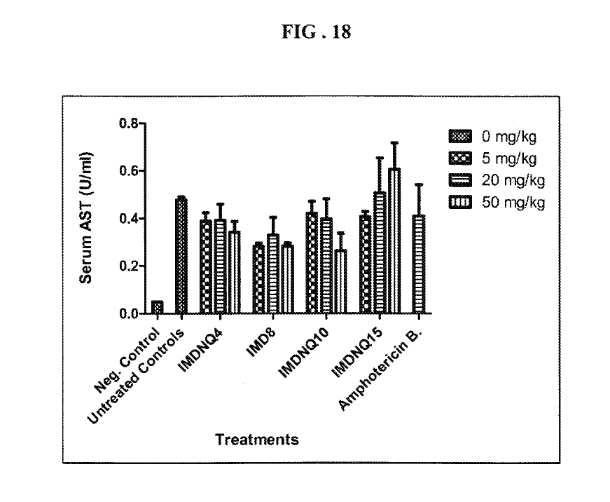

FIG. 18 shows the levels of serum AST in negative controls, treated and untreated samples. For each of the three concentrations (5 mg/kg, 20 mg/kg, and 50 mg/kg), percent increases in AST levels in the four compounds (IMDNQ4, IMD8, IMDNQ10, and IMDNQ15) and Amphotericin B are compared to percent increases in AST levels in untreated controls, which is 89.76%. For IMDNQ4: 5 mg/kg had an 87.40% increase in AST levels, a 2.4% decrease; 20 mg/kg had an 87.48% increase in AST levels, a 2.3% decrease; 50 mg/kg had an 85.65% increase in AST levels, a 4.1% decrease. For IMD8: 5 mg/kg had an 82.69% increase in AST levels, a 7.1% decrease; 20 mg/kg had an 87.48% increase in AST levels, a 2.3% decrease; 50 mg/kg had an 85.65% increase in AST levels, a 4.1% decrease; 20 mg/kg, had an 85.17% increase in AST levels, a 4.6% decrease; 50 mg/kg had an 82.69% increase in AST levels, a 7.1% decrease. For IMDNQ10: 5 mg/kg had an 88.32% increase in AST levels, a 1.4% decrease; 20 mg/kg had an 87.64% increase in AST levels, a 2.1% decrease; 50 mg/kg had an 81.44% increase in AST levels, an 8.3% decrease. For IMDNQ15: 5 mg/kg had an 87.95% increase in AST levels, a 2.0% decrease; 20 mg/kg had a 90.34% increase in AST levels, a 0.58% increase; 50 mg/kg had a 91.91% increase in AST levels, a 2.2% increase. Amphotericin B had an 88.05% increase in AST level, a 1.7% decrease. The determination of AST in serum was assayed as a manifestation of hepatic abnormality, mainly as an increase in the AST level. Usually less than two times the baseline value. The rates of hepatic adverse events (AST) between positive controls and treated groups was not significant. Levels were significant when compared with negative controls (P=0.03). The assay range for this test was 0.096-3.8 U/ml (mean=1.9 U/ml) while all tested samples were <0.606 U/ml.

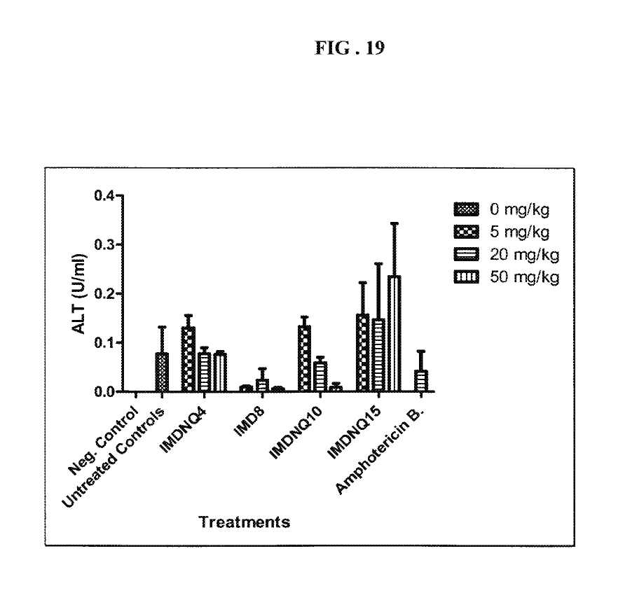

FIG. 19 shows ALT levels IMDNQ4, IMD8, IMDNQ10, IMDNQ15 and Amphotericin B, compared to ALT levels in the uninfected (negative controls), infected but not treated (positive controls), and infected but treated samples. All compounds showed a 100% increase in the level of ALT in scrum compared to the level of ALT in the uninfected samples (negative controls). The determination of ALT in serum was assayed as a manifestation of hepatic abnormality, mainly as an increase in the ALT level. Usually less than two times the baseline value. The rates of hepatic adverse events (ALT) between positive controls (untreated controls) and treated groups was not significant (P=0.09). Levels were not significant when compared with negative controls (P=0.08). The assay range for this method was 0.112-3.00 U/ml (mean=1.6 U/ml) and all tested samples were <0.234 U/ml.

Table 6 is a summary of FIGS. 16, 18 and 19. The Table shows the following data: percent increases in the levels of IgG, serum AST, and serum ALT in the control and untreated control, the four naphthoquinone compounds (IMDNQ4, IMD8, IMDNQ 10, and IMDNQ 15) and Amphotericin B; and p-values for AST and ALT.

TABLE 7
Seroprevalence of IgG ELISAs, AST and ALT of uninfected controls, infected treated
and untreated BALB/c mice. P values were calculated at P = 0.05. P values are
calculated using test samples compared with the negative controls. NS = Not
statistically significant. The assay range for AST was 0.096-3.8 U/ml and ALT
was from 0.112-3.0 U/ml. Reference ranges for AST was 0.16-0.8 U/ml and
ALT was 0.1-0.7 U/ml. All tested samples had values ranging from 0.049-0.606
U/ml (AST) and 0.0-0.234 U/ml (ALT) which were on the very low end of the
curve which is an indication of no damage caused to the liver.

| | Treatments | IgG | Serum AST | | P- | |
|---|---|---|---|---|---|---|
| | Total dose | Increase | Increase | Serum ALT | Values | P-Values |
| Compounds | (mg/kg) | (%) | (%) | Increase (%) | (AST) | (ALT) |
| Control | None | 0 | 0 | 0 | — | — |
| Untreated | None | 20.36 | 89.76 | 100 | 0.0007 | NS |
| Control | | | | | | |
| IMDNQ4 | 5 | 11.34 | 87.40 | 100 | 0.01 | 0.04 |
| | 20 | 6.81 | 87.48 | 100 | 0.04 | 0.02 |
| | 50 | 26.76 | 85.65 | 100 | 0.02 | 0.006 |
| IMD8 | 5 | 20.94 | 82.69 | 100 | 0.003 | 0.04 |
| | 20 | 28.66 | 85.17 | 100 | NS | NS |
| | 50 | 23.16 | 82.69 | 100 | 0.003 | NS |
| IMDNQ10 | 5 | 27.96 | 88.32 | 100 | 0.02 | 0.02 |
| | 20 | 21.05 | 87.64 | 100 | NS | 0.04 |
| | 50 | 22.61 | 81.44 | 100 | NS | NS |
| IMDNQ15 | 5 | 21.79 | 87.95 | 100 | 0.004 | NS |
| | 20 | 14.12 | 90.34 | 100 | NS | NS |
| | 50 | 30.70 | 91.91 | 100 | 0.04 | NS |
| Am. B. | 20 | 24.74 | 88.05 | 100 | NS | NS |

The data in Table 7 are as follows: for each of the three concentrations (5 mg/kg, 20 mg/kg, and 50 mg/kg) levels of serum AST and serum ALT in all test specimens; and percent change in the levels of serum AST and serum ALT in IMDNQ4, IMD8, IMDNQ10, IMDNQ15, and Amphotericin B, compared to the levels of serum AST and serum ALT in untreated controls. As shown in Table 8, a downward arrow indicates a decrease and an upward arrow indicates an increase in the levels of serum AST and serum ALT in the five test specimens compared the levels in untreated controls.

Compared to the untreated controls, for a 14.4% change in the level of serum AST in Amphotericin B, relative changes in the levels of AST in the four compounds were as follows: (1) IMDNQ4: 5 mg/kg had an 18.8% decrease, 20 mg/kg had an 18.2% decrease, and 50 mg/kg had a 28.6% decrease. (2) IMD8: 5 mg/kg had a 40.9% decrease, 20 mg/kg had a 30.9% decrease, and 50 mg/kg had a 40.9% decrease. (3) IMDNQ10: 5 mg/kg had a 12.3% decrease, 20 mg/kg had a 17.1% decrease, and 50 mg/kg had a 44.98% decrease. (4) IMDNQ15: 5 mg/kg had a 15.0% decrease, 20 mg/kg had a 5.5% increase, and 50 mg/kg had a 21.0% decrease.

Compared to the untreated controls, for a 47.4% change in the level of serum ALT in Amphotericin B, relative changes in the levels of ALT in the four compounds were as follows: (1) IMDNQ4: 5 mg/kg had a 40.0% increase, 20 mg/kg had a 0% increase, and 50 mg/kg had a 2.6% decrease. (2) IMD8: 5 mg/kg had an 11.2% decrease, 20 mg/kg had a 69.2% decrease, and 50 mg/kg had an 893% decrease. (3) IMDNQ10: 5 g/kg had a 41.4% increase, 20 mg/kg had a 24.4% decrease, and 50 mg/kg had an 89.7% decrease. (4) IMDNQ15: 5 mg/kg had a 50.0% increase, 20 mg/kg had a 46.6% increase, and 50 mg/kg had a 66.7% increase.

TABLE 8
Serum AST and ALT of infected treated and untreated BALB/c mice. Values are U/ml
and include means ± S.E. (Standard Error). The reference range for AST was
0.096-3.8 U/ml and ALT Was from 0.112-3.0 U/ml. All tested samples had values ranging
fiom 0.049-0.606 U/ml (AST) and 0.0-0.234 U/ml (ALT) which were on the very low
end of the curve; this is an indication of no damage caused to the liver.

| | | | Serum AST | | Serum ALT |
|---|---|---|---|---|---|
| | Treatments | | (%) Compared | | (%) Compared |
| | Total dose | Serum AST | with Untreated | Serum ALT | with Untreated |
| Compounds | (mg/kg) | (U/ml) | Controls | (U/ml) | Controls |
| Untreated | None | 0.479 ± 0.02 | 0 | 0.078 ± 0.08 | 0 |
| Control | | | | | |
| IMDNQ4 | 5 | 0.389 ± 0.05 | 18.8 ↓ | 0.131 ± 0.04 | 40 ↑ |
| | 20 | 0.392 ± 0.10 | 18.2 ↓ | 0.078 ± 0.02 | 0 |
| | 50 | 0.342 ± 0.06 | 28.6 ↓ | 0.076 ± 0.01 | 2.6 ↓ |
| IMD8 | 5 | 0.283 ± 0.02 | 40.9 ↓ | 0.010 ± 0.00 | 11.2 ↓ |
| | 20 | 0.331 ± 0.10 | 30.9 ↓ | 0.024 ± 0.03 | 69.2 ↓ |
| | 50 | 0.283 ± 0.02 | 40.9 ↓ | 0.006 ± 0.00 | 89.7 ↓ |
| IMDNQ10 | 5 | 0.420 ± 0.07 | 12.3 ↓ | 0.133 ± 0.03 | 41.4 ↑ |
| | 20 | 0.397 ± 0.12 | 17.1 ↓ | 0.059 ± 0.02 | 24.4 ↓ |
| | 50 | 0.264 ± 0.11 | 44.9 ↓ | 0.008 ± 0.01 | 89.7 ↓ |
| IMDNQ15 | 5 | 0.407 ± 0.03 | 15.0 ↓ | 0.156 ± 0.09 | 50.0 ↑ |
| | 20 | 0.507 ± 0.21 | 5.5 ↑ | 0.146 ± 0.16 | 46.6 ↑ |
| | 50 | 0.606 ± 0.16 | 21.0 ↑ | 0.234 ± 0.15 | 66.7 ↑ |
| Am. B. | 20 | 0.410 ± 0.19 | 14.4 ↓ | 0.041 ± 0.06 | 47.4 ↓ |

Therefore, the therapeutic effects of these compounds are not only comparable with that of amphotericin B, but are even more effective in the reduction of parasite burden compared to Amphotericin B.

### REFERENCES

The complete disclosure of each reference hereinbelow listed is incorporated by reference.
Bakare O., Ashendel C. L., Peng H., Zalkow L. H., Burgess E. M. (2003). Synthesis and MEK1 inhibitory activities of imido-substituted 2-chloro-1,4-naphthoquinones. Bioorganic and Medicinal Chemistry; 11:3165-3170.
BERMAN, J D. (1997). New England Journal of Medicine, 322, 16±21.
Bern C., Maguire J. H., Alvar J. (2008). Complexities of assessing the disease burden attributable to leimaniasis. PLoS Neglected Tropical Diseases; 2(10):e313.
Burns et al., Molecular characterization of a kinesin-related antigen of Leishmania chagasi that detects specific antibody in African and American visceral leishmaniasis, (1993) Proc. Natl. Acad. Sci. USA, 90:775-779.
CDC: Leishmania FAQs. Global Health-Division of Parasitic Diseases and Malaria. http://www.cdc.gov/parasites/leishmaniasis/gen_info/faqs.html.
CDC: Leishmaniasis, Cutaneous (CL). Herwaldt B. L. and Magill A. J. http://wwwnc.cdc.gov/travel/yellowbook/2010/chapter-5/cutaneous-Lcishmaniasis.aspx. Retrieved on Apr. 8, 2011.
CDC: Parasites and Health: Leishmaniasis. (2009). http://www.dpd.cdc.gov/dpdx/html/leishmaniasis.htm; Retrieved on Sep. 20, 2010.
Croft S. L. & Yardley V. (2002). Chemotherapy of leishmaniasis. Current Pharmaceutical Design; 8:319-342.
Croft S. L. & Coombs G. H. (2003). Leishmaniasis-current chemotherapy and recent advances in the search for novel drugs. Trends in Parasitology; 19:502-508.
Croft, S. L., Evans A. T., Neal R. A. (1985). The activity of plumbagin and other electron carriers against Leishmania donovani and Leishmania mexicana. Annals of Tropical Medicine and Parasitology; 79:651-653.
Davidson R N, Russo R. Bull World Health Organ (1994); 72:931-6. 5. Relapse of visceral leishmaniasis in patients who were coinfected with human immunodeficiency.
Desjeux P. (2004). Leishmaniasis: current situation and new perspectives. Comparative Immunology, Microbiology and Infectious disease; 27:305-318.
Gradoni L., Soteriadou K., Louzir H., Dakkak A., Toz S. O., Jaffe C., Dedet J. P., Campino L., Canavate C., Dujardin J. C. (2008). Drug regimens for visceral leishmaniasis in mediterranean countries. Tropical Medicine and International Health; 13(10):1272-1276.
Guderian R H, Chico M E, Rogers M D et al. Trop Med Hyg 1991; 45:92-97
Hudson A. T. (1993). Atovaquone—a novel broad-spectrum anti-infective drug. Parasitology Today; 9:66-68.
Mallick et al., The influence of surface modification on the cytotoxicity of PAMAM dendrimers. Int. J. Pharm, 252 (2003), pp. 263-266.
Mikus, J., and D. Steverding. (2000). A simple colorimetric method to screen drug cytotoxicity against Leishmania using the dye Alamar Blue. Parasitol. Int. 48: 265-269.
Modabber F., Buffet P. A., Torreele E., Milon G., Croft S. L. (2007). Consultative meeting to develop a strategy for treatment of cutaneous leishmaniasis. Institute Pasteur, Paris. 13-15 June, 2006. Kinetoplastid Biology and Disease; 6:3.
Monzote L. (2009). Current treatment of leishmaniasis: A review. The Open Antimicrobial Agents Journal; 1:9-19.
Reitman, S. and Frankel, S. A. (1957) A colourimetric method for the determination of serum oxaloaetic and glutamic pyruvic transaminases. Am. J. Clin. Pathol. 28, 56-63.
Scala F., Fattorusso E., Menna M., Taglialatela-Scafati O., Tiemey M., Kaiser M., and Tasdemir D. (2010) Bromopyrrole alkaloids as lead compounds against protozoan parasites. Marine Drugs; 8:2162-2174.
Sen et al., 2004. Camptothecin induced mitochondrial dysfunction leading to programmed cell death in unicellular hemoflagellate Leishmania donovani. Cell Death and Differentiation (2004) 11, 924-936. doi:10.1038/sj.cdd.4401435 Published online 30 Apr. 2004.
Singh S, Sivakumar R. Challenges and new dis-coveries in the treatment of leishmaniasis. J In-fect Chemother 2004; 10:307-15.
Stauber et al., 1958. Stauber, L. A. (1958) Host resistance to the Khartoum strain of Leishmania donovani. Rice Inst. Pamph. 45,80.
Sundar S., More D. K., Singh M. K., Singh V. P., Sharma S., Makharia A., Kumar P. C., Murray H. W. (2000). Failure of pentavalent antimony in visceral leishmaniasis in India: report from the center of the Indian epidemic. Clinical Infectious Diseases; 31:1104-1106.
Todaro G J, Green H. Quantitative studies of the growth of mouse embryo cells in culture and their development into established lines. J. Cell Biol. 17: 299-313, (1963).
Tripathi P., Singh V., and Naik S. (2007). Immune response to leishmania: paradox rather than paradigm. FEMS Immunology and Medical Microbiology; 51:229-242.
Vannier-Santos M. A., Martiny A., De Souza W. (2002). Cell biology of Leishmania spp.: invading and evading. Current Pharmaceutical Design; 8:297-318.
Vidyashankar C. & Agrawal R. (2009). Pediatric Leishmaniasis. Emedicine.medscape.com (http://emedicine.medscape.com/article/998804-print); Retrived on Mar. 7, 2011.
World Health Organization Action in Afghanistan aims to control debilitating leishmaniasis (2004); By Cheng M. http://www.who.int/mediacentre/news/releases/2004/pr55/en/; Retrieved on Mar. 7, 2011.
WHO: control of neglected tropical disease is feasible. Renewed engagement to scale-up integrated interventions announced. (2010) By Moloo A. & Rio F. http://www.who.int/mediacentre/news/releases/2010/ntd_{—}20101014/en/; Retrieved on Mar. 7, 2011.
Zijlstra E E, Daifalla N S, Kager P A, Khalil E A, Hassan AME, Reed S G, Ghalib H W, 1998. rK39 enzyme-linked immunosorbent assay for diagnosis of Leishmania donovani infection. Clin Diagn Lab Immunol 5: 717-720.

1. A method selected from the group consisting of:
(a) a method of inhibiting proliferation of Leishmania parasites in a patient for prophylaxis or in a patient in need of treatment which comprises administering to said patient an anti-Leishmanial effective amount of an imido-substituted 1,4-naphthoquinone represented by the general formula: 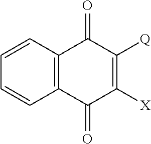
wherein X is hydrogen, halogen, alkoxy (cyclic or alicyclic), trifluoro methyl, aryloxy, or benzyloxy; and Q represents the imido-substitutent bonded to the 1,4-naphthoquinone moiety through the imido nitrogen;
(b) a method of inhibiting Leishmania parasite growth or proliferation in a patient in need of treatment comprising the step of administering to said patient, in an amount effective for anti-Leishmanial activity, an imido-substituted 1,4-naphthoquinone represented by the general formula: 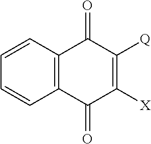
wherein X is hydrogen, halogen, alkoxy (cyclic or alicyclic), trifluoro methyl, aryloxy, or benzyloxy; and Q represents the imido-substitutent bonded to the 1,4-naphthoquinone moiety through the imido nitrogen; and
(c) a method of treating a patient for prophylaxis or to a patient in need of treatment for leishmaniasis comprising the step of administering to said patient a therapeutically effective amount of an imido-substituted 1,4-naphthoquinone represented by the general formula: 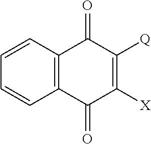
wherein X is hydrogen, halogen, alkoxy (cyclic or alicyclic), trifluoro methyl, aryloxy, or benzyloxy; and Q represents the imido-substitutent bonded to the 1,4-naphthoquinone moiety through the imido nitrogen.

2. The method according to claim 1, wherein the imido-substituted 1,4-naphthoquinone has in vitro toxicity against Leishmania donovani greater than Amphotericin B.

3. The method according to claim 1, wherein the imido-substituted 1,4-naphthoquinone has an in vitro selectivity index greater than Amphotericin B.

4. The method according to claim 1, wherein the imido-substituted 1,4-naphthoquinone has an IC₅₀ value against Leishmania donovani promastigotes and amastigotes lower than Amphotericin B.

5. (canceled)

6. (canceled)

7. A method according to claim 1, wherein Q is represented by the formula: 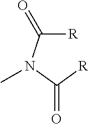
wherein in Q each R is, independently, a substituted or unsubstituted hydrocarbon, provided that one R can, optionally, be hydrogen, and provided that, optionally, R can include at least one hetero atom.

8. The method according to claim 1, wherein Q is represented by the formula 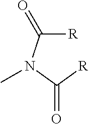
wherein each R is cyclic or acyclic, or the R groups are bonded together.

9. The method according to claim 1, wherein each R is aryl, halo-substituted aryl, aliphatic, halo-substituted aliphatic, or alkenyl.

10. (canceled)

11. The method according to claim 1, wherein Q is an aryl-imido substituent.

12. The method according to claim 1, wherein each R is aryl, optionally having halogen substitution.

13. The method according to claim 1, wherein the imido-substituted 1,4-naphthoquinone is represented by the formula: 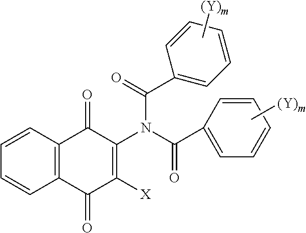
wherein X is hydrogen, halogen, alkoxy (cyclic or alicyclic), trifluoro methyl, aryloxy, or benzyloxy, each Y, independently, represents halogen, alkoxy (cyclic or alicyclic), trifluoro methyl or alkyl, and each m, independent of the other, is 0, 1, 2, 3, 4 or 5.

14. The method according to claim 13, wherein the imido-substituted 1,4-naphthoquinone is represented by the formula: 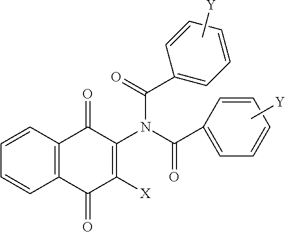
wherein X is hydrogen, halogen, alkoxy (cyclic or alicyclic), trifluoro methyl, aryloxy, or benzyloxy, and each Y, independently, represents hydrogen, halogen, alkyl, or alkoxy.

15. The method according to claim 14, wherein Y is a meta-substitutent.

16. The method according to claim 14, wherein Y is an ortho-substitutent.

17. The method according to claim 14, wherein Y is a para-substitutent.

18. The method according to claim 13, wherein Y is bromo, chloro or fluoro.

19. The method according to claim 13, wherein X is chloro.

20. The method according to claim 19, wherein X is chloro and Y is chloro.

21. The method according to claim 1, wherein the imido-substituted 1,4-naphthoquinone is selected from the group consisting of IMDNQ2, IMDNQ3 and IMDNQ4.

22. The method according to claim 7, wherein R is a C₁-C₁₀ alkyl, optionally having halogen substitution.

23. The method according to claim 22, wherein R has terminal halo-substitution.

24. The method according to claim 1, wherein the imido-substituted 1,4-naphthoquinone is selected from the group consisting of IMDNQ1, IMDNQ2, IMDNQ3, IMDNQ4, IMDNQ5, IMDNQ6, IMDNQ9, IMDNQ10, IMDNQ12, IMDNQ13, IMDNQ14 and IMDNQ15.

25. The method according to claim 7, wherein each R is alkyl or cyclo alkyl.

26. The method according to claim 1, wherein Q is represented by the formula 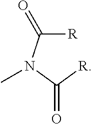
wherein the R groups bond together to form an alkylene bridge, optionally including a hetero atom, and optionally substituted.

27. The method according to claim 26, wherein the R groups bond together to form an alkylene bridge including a hetero atom.

28. The method according to claim 27, wherein the hetero atom is an oxygen atom.

29. The method according to claim 24, wherein the imido-substituted 1,4-naphthoquinone is IMDNQ15.

30. The method according to claim 1, wherein the imido-substituted 1,4-naphthoquinone is a phthalimidyl-substituted 1,4-naphthoquinone.

32. The method according to claim 1, wherein X is halogen.

33. The method according to claim 32, wherein X is chloro.

34. The method according to claim 32, wherein X is bromo.

35. A pharmaceutical composition used for inhibiting proliferation of Leishmania parasites in a patient for prophylaxis or to a patient in need of treatment for Leishmaniasis comprising an imido-substituted 1,4-naphthoquinone represented by the general formula: 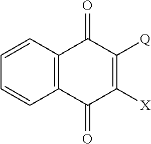
wherein X is halogen; and Q represents an imido-substitutent bonded to the 1,4-naphthoquinone moiety through the imido nitrogen; and a pharmaceutically acceptable carrier or adjuvant; or pharmaceutical composition comprised of an imido-substituted 1,4-naphthoquinone represented by the general formula: 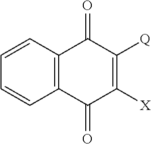
wherein X is hydrogen, halogen, alkoxy (cyclic or alicyclic), trifluoro methyl, aryloxy, or benzyloxy; and Q represents an imido-substitutent bonded to the 1,4-naphthoquinone moiety through the imido nitrogen; and a pharmaceutically acceptable carrier or adjuvant, wherein said pharmaceutical composition is formulated for use in inhibiting proliferation of a Leishmania parasite in a patient for prophylaxis or to a patient in need of treatment for Leishmaniasis.

36. The pharmaceutical composition according to claim 35, wherein X is chlorine.

37. The pharmaceutical composition according to claim 35, wherein X is bromine.

38. (canceled)
